SEQUENCE LISTING (54 sequences)

SEQ ID NO: 1 - RNA, 100 nt, synthetic construct
  source 1..100: mol_type = other RNA
cttgccccacagggcagtaagttttagagctagaaatagcaagttaaaataaggctagtccgttatcaacttgaaaaagtggcaccgagtcggtgctttt

SEQ ID NO: 2 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 97..99: mod_base = OTHER; note = 2'-O-methyl-3'-phosphorothioate nucleotide

SEQ ID NO: 3 - RNA, 99 nt, synthetic construct
  source 1..99: mol_type = other RNA
  modified_base 1..3: mod_base = OTHER; note = 2'-O-methyl-3'-phosphorothioate nucleotide
  modified_base 98: mod_base = OTHER; note = 2'-O-methyl-3'-phosphonoacetate nucleotide
cttgccccacagggcagtaagttttagagctagaaatagcaagttaaaataaggctagtccgttatcaacttgaaaaagtggcaccgagtcggtgcttt

SEQ ID NO: 4 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 99: mod_base = OTHER; note = 2'-O-methyl-3'-phosphonoacetate nucleotide

SEQ ID NO: 5 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 98..99: mod_base = OTHER; note = 2'-O-methyl-3'-phosphonoacetate nucleotide

SEQ ID NO: 6 - RNA, 101 nt, synthetic construct
  source 1..101: mol_type = other RNA
  modified_base 1..3: mod_base = OTHER; note = 2'-O-methyl-3'-phosphorothioate nucleotide
  modified_base 98..100: mod_base = OTHER; note = 2'-O-methyl-3'-phosphonoacetate nucleotide
cttgccccacagggcagtaagttttagagctagaaatagcaagttaaaataaggctagtccgttatcaacttgaaaaagtggcaccgagtcggtgcttttt

SEQ ID NO: 7 - RNA, 102 nt, synthetic construct
  source 1..102: mol_type = other RNA
  modified_base 1..3: mod_base = OTHER; note = 2'-O-methyl-3'-phosphorothioate nucleotide
  modified_base 99..101: mod_base = OTHER; note = 2'-O-methyl-3'-phosphonoacetate nucleotide
cttgccccacagggcagtaagttttagagctagaaatagcaagttaaaataaggctagtccgttatcaacttgaaaaagtggcaccgagtcggtgctttttt

SEQ ID NO: 8 - RNA, 102 nt, synthetic construct; identical to SEQ ID NO: 7
  source 1..102: mol_type = other RNA
  modified_base 1..3: mod_base = OTHER; note = 2'-O-methyl-3'-phosphorothioate nucleotide
  modified_base 98..101: mod_base = OTHER; note = 2'-O-methyl-3'-phosphonoacetate nucleotide

SEQ ID NO: 9 - RNA, 103 nt, synthetic construct
  source 1..103: mol_type = other RNA
  modified_base 1..3: mod_base = OTHER; note = 2'-O-methyl-3'-phosphorothioate nucleotide
  modified_base 99..103: mod_base = OTHER; note = 2'-O-methyl-3'-phosphonoacetate nucleotide
cttgccccacagggcagtaagttttagagctagaaatagcaagttaaaataaggctagtccgttatcaacttgaaaaagtggcaccgagtcggtgcttttttt

SEQ ID NO: 10 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 97..99: mod_base = OTHER; note = 2'-O-methyl-3'-phosphorothioate nucleotide
  modified_base 5: mod_base = OTHER; note = 2'-O-methyl-3'-phosphonoacetate nucleotide

SEQ ID NO: 11 - RNA, 99 nt, synthetic construct; identical to SEQ ID NO: 3
  source 1..99: mol_type = other RNA
  modified_base 1..3: mod_base = OTHER; note = 2'-O-methyl-3'-phosphorothioate nucleotide
  modified_base 5: mod_base = OTHER; note = 2'-O-methyl-3'-phosphonoacetate nucleotide
  modified_base 98: mod_base = OTHER; note = 2'-O-methyl-3'-phosphonoacetate nucleotide

SEQ ID NO: 12 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 5: mod_base = OTHER; note = 2'-O-methyl-3'-phosphonoacetate nucleotide
  modified_base 98..99: mod_base = OTHER; note = 2'-O-methyl-3'-phosphonoacetate nucleotide

SEQ ID NO: 13 - RNA, 101 nt, synthetic construct; identical to SEQ ID NO: 6
  source 1..101: mol_type = other RNA
  modified_base 1..3: mod_base = OTHER; note = 2'-O-methyl-3'-phosphorothioate nucleotide
  modified_base 5: mod_base = OTHER; note = 2'-O-methyl-3'-phosphonoacetate nucleotide
  modified_base 98..100: mod_base = OTHER; note = 2'-O-methyl-3'-phosphonoacetate nucleotide

SEQ ID NO: 14 - RNA, 102 nt, synthetic construct; identical to SEQ ID NO: 7
  source 1..102: mol_type = other RNA
  modified_base 1..3: mod_base = OTHER; note = 2'-O-methyl-3'-phosphorothioate nucleotide
  modified_base 5: mod_base = OTHER; note = 2'-O-methyl-3'-phosphonoacetate nucleotide
  modified_base 98..101: mod_base = OTHER; note = 2'-O-methyl-3'-phosphonoacetate nucleotide

SEQ ID NO: 15 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 97..99: mod_base = OTHER; note = 2'-O-methyl-3'-phosphorothioate nucleotide
  modified_base 11: mod_base = OTHER; note = 2'-O-methyl-3'-phosphonoacetate nucleotide

SEQ ID NO: 16 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 11: mod_base = OTHER; note = 2'-O-methyl-3'-phosphonoacetate nucleotide
  modified_base 99: mod_base = OTHER; note = 2'-O-methyl-3'-phosphonoacetate nucleotide

SEQ ID NO: 17 - RNA, 100 nt, synthetic construct; identical to SEQ ID NO: 1
  source 1..100: mol_type = other RNA
  modified_base 1..3: mod_base = OTHER; note = 2'-O-methyl-3'-phosphorothioate nucleotide
  modified_base 11: mod_base = OTHER; note = 2'-O-methyl-3'-phosphonoacetate nucleotide
  modified_base 98..99: mod_base = OTHER; note = 2'-O-methyl-3'-phosphonoacetate nucleotide

SEQ ID NO: 18 - RNA, 102 nt, synthetic construct; identical to SEQ ID NO: 7
  source 1..102: mol_type = other RNA
  modified_base 1..3: mod_base = OTHER; note = 2'-O-methyl-3'-phosphorothioate nucleotide
  modified_base 11: mod_base = OTHER; note = 2'-O-methyl-3'-phosphonoacetate nucleotide
  modified_base 99..101: mod_base = OTHER; note = 2'-O-methyl-3'-phosphonoacetate nucleotide

SEQ ID NO: 19 - RNA, 103 nt, synthetic construct; identical to SEQ ID NO: 9
  source 1..103: mol_type = other RNA
  modified_base 1..3: mod_base = OTHER; note = 2'-O-methyl-3'-phosphorothioate nucleotide
  modified_base 11: mod_base = OTHER; note = 2'-O-methyl-3'-phosphonoacetate nucleotide
  modified_base 99..102: mod_base = OTHER; note = 2'-O-methyl-3'-phosphonoacetate nucleotide

SEQ ID NO: 20 - RNA, 163 nt, synthetic construct
  source 1..163: mol_type = other RNA
cttgccccacagggcagtaagttttagagctaggccaacatgaggatcacccatgtctgcagggcctagcaagttaaaataaggctagtccgttatcaacttggccaacatgaggatcacccatgtctgcagggccaagtggcaccgagtcggtgcttttttt

SEQ ID NO: 21 - RNA, 163 nt, synthetic construct; identical to SEQ ID NO: 20
  source 1..163: mol_type = other RNA
  modified_base 1..3: mod_base = OTHER; note = 2'-O-methyl-3'-phosphorothioate nucleotide
  modified_base 162: mod_base = OTHER; note = 2'-O-methyl-3'-phosphonoacetate nucleotide

SEQ ID NO: 22 - RNA, 163 nt, synthetic construct; identical to SEQ ID NO: 20
  source 1..163: mol_type = other RNA
  modified_base 1..3: mod_base = OTHER; note = 2'-O-methyl-3'-phosphorothioate nucleotide
  modified_base 161..162: mod_base = OTHER; note = 2'-O-methyl-3'-phosphonoacetate nucleotide

SEQ ID NO: 23 - RNA, 163 nt, synthetic construct; identical to SEQ ID NO: 20
  source 1..163: mol_type = other RNA
  modified_base 1..3: mod_base = OTHER; note = 2'-O-methyl-3'-phosphorothioate nucleotide
  modified_base 160..162: mod_base = OTHER; note = 2'-O-methyl-3'-phosphonoacetate nucleotide

SEQ ID NO: 24 - RNA, 163 nt, synthetic construct; identical to SEQ ID NO: 20
  source 1..163: mol_type = other RNA
  modified_base 1..3: mod_base = OTHER; note = 2'-O-methyl-3'-phosphorothioate nucleotide
  modified_base 159..162: mod_base = OTHER; note = 2'-O-methyl-3'-phosphonoacetate nucleotide

SEQ ID NO: 25 - RNA, 124 nt, synthetic construct
  source 1..124: mol_type = other RNA
  modified_base 1..3: mod_base = OTHER; note = 2'-O-methyl-3'-phosphorothioate nucleotide
gagtccgagcagaagaagaagttttagagctagaaatagcaagttaaaataaggctagtccgttatcaacttgaaaaagtggcaccgagtcggtgcatgggagcacttcttcttctgctcggac

SEQ ID NO: 26 - RNA, 124 nt, synthetic construct; identical to SEQ ID NO: 25
  source 1..124: mol_type = other RNA
  modified_base 1..3: mod_base = OTHER; note = 2'-O-methyl-3'-phosphorothioate nucleotide
  modified_base 121..123: mod_base = OTHER; note = 2'-O-methyl-3'-phosphorothioate nucleotide

SEQ ID NO: 27 - RNA, 124 nt, synthetic construct; identical to SEQ ID NO: 25
  source 1..124: mol_type = other RNA
  modified_base 1..3: mod_base = OTHER; note = 2'-O-methyl-3'-phosphorothioate nucleotide
  modified_base 123: mod_base = OTHER; note = 2'-O-methyl-3'-phosphonoacetate nucleotide

SEQ ID NO: 28 - RNA, 124 nt, synthetic construct; identical to SEQ ID NO: 25
  source 1..124: mol_type = other RNA
  modified_base 1..3: mod_base = OTHER; note = 2'-O-methyl-3'-phosphorothioate nucleotide
  modified_base 122..123: mod_base = OTHER; note = 2'-O-methyl-3'-phosphonoacetate nucleotide

SEQ ID NO: 29 - RNA, 124 nt, synthetic construct; identical to SEQ ID NO: 25
  source 1..124: mol_type = other RNA
  modified_base 1..3: mod_base = OTHER; note = 2'-O-methyl-3'-phosphorothioate nucleotide
  modified_base 121..123: mod_base = OTHER; note = 2'-O-methyl-3'-phosphonoacetate nucleotide

SEQ ID NO: 30 - RNA, 100 nt, synthetic construct
  source 1..100: mol_type = other RNA
  modified_base 1..3: mod_base = OTHER; note = 2'-O-methyl-3'-phosphorothioate nucleotide
  modified_base 97..99: mod_base = OTHER; note = 2'-O-methyl-3'-phosphorothioate nucleotide
gccgtttgtactttgtcctcgttttagagctagaaatagcaagttaaaataaggctagtccgttatcaacttgaaaaagtggcaccgagtcggtgctttt

SEQ ID NO: 31 - RNA, 129 nt, synthetic construct
  source 1..129: mol_type = other RNA
  modified_base 1..3: mod_base = OTHER; note = 2'-O-methyl-3'-phosphorothioate nucleotide
gcattttcaggaggaagcgagttttagagctagaaatagcaagttaaaataaggctagtccgttatcaacttgaaaaagtggcaccgagtcggtgctgtctgaagccatccatgcttcctcctgaaaat

SEQ ID NO: 32 - RNA, 129 nt, synthetic construct; identical to SEQ ID NO: 31
  source 1..129: mol_type = other RNA
  modified_base 1..3: mod_base = OTHER; note = 2'-O-methyl-3'-phosphorothioate nucleotide
  modified_base 126..128: mod_base = OTHER; note = 2'-O-methyl-3'-phosphorothioate nucleotide

SEQ ID NO: 33 - RNA, 129 nt, synthetic construct; identical to SEQ ID NO: 31
  source 1..129: mol_type = other RNA
  modified_base 1..3: mod_base = OTHER; note = 2'-O-methyl-3'-phosphorothioate nucleotide
  modified_base 128: mod_base = OTHER; note = 2'-O-methyl-3'-phosphonoacetate nucleotide

SEQ ID NO: 34 - RNA, 129 nt, synthetic construct; identical to SEQ ID NO: 31
  source 1..129: mol_type = other RNA
  modified_base 1..3: mod_base = OTHER; note = 2'-O-methyl-3'-phosphorothioate nucleotide
  modified_base 127..128: mod_base = OTHER; note = 2'-O-methyl-3'-phosphonoacetate nucleotide

SEQ ID NO: 35 - RNA, 129 nt, synthetic construct; identical to SEQ ID NO: 31
  source 1..129: mol_type = other RNA
  modified_base 1..3: mod_base = OTHER; note = 2'-O-methyl-3'-phosphorothioate nucleotide
  modified_base 126..128: mod_base = OTHER; note = 2'-O-methyl-3'-phosphonoacetate nucleotide

SEQ ID NO: 36 - RNA, 100 nt, synthetic construct
  source 1..100: mol_type = other RNA
  modified_base 1..3: mod_base = OTHER; note = 2'-O-methyl-3'-phosphorothioate nucleotide
  modified_base 97..99: mod_base = OTHER; note = 2'-O-methyl-3'-phosphorothioate nucleotide
atgaagcactgtgggtacgagttttagagctagaaatagcaagttaaaataaggctagtccgttatcaacttgaaaaagtggcaccgagtcggtgctttt

SEQ ID NO: 37 - RNA, 100 nt, synthetic construct
  source 1..100: mol_type = other RNA
  modified_base 1..3: mod_base = OTHER; note = 2'-O-methyl-3'-phosphorothioate nucleotide
  modified_base 97..99: mod_base = OTHER; note = 2'-O-methyl-3'-phosphorothioate nucleotide
tggtaatgatggcttcaacagttttagagctagaaatagcaagttaaaataaggctagtccgttatcaacttgaaaaagtggcaccgagtcggtgctttt

SEQ ID NO: 38 - RNA, 100 nt, synthetic construct; identical to SEQ ID NO: 37
  source 1..100: mol_type = other RNA
  modified_base 1..3: mod_base = OTHER; note = 2'-O-methyl-3'-phosphorothioate nucleotide
  modified_base 98..99: mod_base = OTHER; note = 2'-O-methyl-3'-phosphonoacetate nucleotide

SEQ ID NO: 39 - RNA, 102 nt, synthetic construct
  source 1..102: mol_type = other RNA
  modified_base 1..3: mod_base = OTHER; note = 2'-O-methyl-3'-phosphorothioate nucleotide
  modified_base 99..101: mod_base = OTHER; note = 2'-O-methyl-3'-phosphonoacetate nucleotide
tggtaatgatggcttcaacagttttagagctagaaatagcaagttaaaataaggctagtccgttatcaacttgaaaaagtggcaccgagtcggtgctttttt

SEQ ID NO: 40 - RNA, 103 nt, synthetic construct
  source 1..103: mol_type = other RNA
  modified_base 1..3: mod_base = OTHER; note = 2'-O-methyl-3'-phosphorothioate nucleotide
  modified_base 99..102: mod_base = OTHER; note = 2'-O-methyl-3'-phosphonoacetate nucleotide
tggtaatgatggcttcaacagttttagagctagaaatagcaagttaaaataaggctagtccgttatcaacttgaaaaagtggcaccgagtcggtgcttttttt

SEQ ID NO: 41 - RNA, 126 nt, synthetic construct
  source 1..126: mol_type = other RNA
  modified_base 1..3: mod_base = OTHER; note = 2'-O-methyl-3'-phosphorothioate nucleotide
  modified_base 123..125: mod_base = OTHER; note = 2'-O-methyl-3'-phosphorothioate nucleotide
gagtccgagcagaagaagaagttttagagctagaaatagcaagttaaaataaggctagtccgttatcaacttgaaaaagtggcaccgagtcggtgcatgggagcacttcttcttctgctcggactt

SEQ ID NO: 42 - RNA, 128 nt, synthetic construct
  source 1..128: mol_type = other RNA
  modified_base 1..3: mod_base = OTHER; note = 2'-O-methyl-3'-phosphorothioate nucleotide
  modified_base 125..127: mod_base = OTHER; note = 2'-O-methyl-3'-phosphorothioate nucleotide
gagtccgagcagaagaagaagttttagagctagaaatagcaagttaaaataaggctagtccgttatcaacttgaaaaagtggcaccgagtcggtgcatgggagcacttcttcttctgctcggactttt

SEQ ID NO: 43 - RNA, 126 nt, synthetic construct; identical to SEQ ID NO: 41
  source 1..126: mol_type = other RNA
  modified_base 1..3: mod_base = OTHER; note = 2'-O-methyl-3'-phosphorothioate nucleotide
  modified_base 125: mod_base = OTHER; note = 2'-O-methyl-3'-phosphonoacetate nucleotide

SEQ ID NO: 44 - RNA, 126 nt, synthetic construct; identical to SEQ ID NO: 41
  source 1..126: mol_type = other RNA
  modified_base 1..3: mod_base = OTHER; note = 2'-O-methyl-3'-phosphorothioate nucleotide
  modified_base 124..125: mod_base = OTHER; note = 2'-O-methyl-3'-phosphonoacetate nucleotide

SEQ ID NO: 45 - RNA, 127 nt, synthetic construct
  source 1..127: mol_type = other RNA
  modified_base 1..3: mod_base = OTHER; note = 2'-O-methyl-3'-phosphorothioate nucleotide
  modified_base 125..126: mod_base = OTHER; note = 2'-O-methyl-3'-phosphonoacetate nucleotide
gagtccgagcagaagaagaagttttagagctagaaatagcaagttaaaataaggctagtccgttatcaacttgaaaaagtggcaccgagtcggtgcatgggagcacttcttcttctgctcggacttt

SEQ ID NO: 46 - RNA, 128 nt, synthetic construct; identical to SEQ ID NO: 42
  source 1..128: mol_type = other RNA
  modified_base 1..3: mod_base = OTHER; note = 2'-O-methyl-3'-phosphorothioate nucleotide
  modified_base 125..127: mod_base = OTHER; note = 2'-O-methyl-3'-phosphonoacetate nucleotide

SEQ ID NO: 47 - RNA, 128 nt, synthetic construct; identical to SEQ ID NO: 42
  source 1..128: mol_type = other RNA
  modified_base 1..3: mod_base = OTHER; note = 2'-O-methyl-3'-phosphorothioate nucleotide
  modified_base 126..127: mod_base = OTHER; note = 2'-O-methyl-3'-phosphonoacetate nucleotide

SEQ ID NO: 48 - RNA, 128 nt, synthetic construct; identical to SEQ ID NO: 42
  source 1..128: mol_type = other RNA
  modified_base 1..3: mod_base = OTHER; note = 2'-O-methyl-3'-phosphorothioate nucleotide
  modified_base 127: mod_base = OTHER; note = 2'-O-methyl-3'-phosphonoacetate nucleotide

SEQ ID NO: 49 - RNA, 131 nt, synthetic construct
  source 1..131: mol_type = other RNA
  modified_base 1..3: mod_base = OTHER; note = 2'-O-methyl-3'-phosphorothioate nucleotide
  modified_base 128..130: mod_base = OTHER; note = 2'-O-methyl-3'-phosphorothioate nucleotide
gcattttcaggaggaagcgagttttagagctagaaatagcaagttaaaataaggctagtccgttatcaacttgaaaaagtggcaccgagtcggtgctgtctgaagccatccatgcttcctcctgaaaattt

SEQ ID NO: 50 - RNA, 133 nt, synthetic construct
  source 1..133: mol_type = other RNA
  modified_base 1..3: mod_base = OTHER; note = 2'-O-methyl-3'-phosphorothioate nucleotide
  modified_base 130..132: mod_base = OTHER; note = 2'-O-methyl-3'-phosphorothioate nucleotide
gcattttcaggaggaagcgagttttagagctagaaatagcaagttaaaataaggctagtccgttatcaacttgaaaaagtggcaccgagtcggtgctgtctgaagccatccatgcttcctcctgaaaattttt

SEQ ID NO: 51 - RNA, 131 nt, synthetic construct; identical to SEQ ID NO: 49
  source 1..131: mol_type = other RNA
  modified_base 1..3: mod_base = OTHER; note = 2'-O-methyl-3'-phosphorothioate nucleotide
  modified_base 129..130: mod_base = OTHER; note = 2'-O-methyl-3'-phosphonoacetate nucleotide

SEQ ID NO: 52 - RNA, 133 nt, synthetic construct; identical to SEQ ID NO: 50
  source 1..133: mol_type = other RNA
  modified_base 1..3: mod_base = OTHER; note = 2'-O-methyl-3'-phosphorothioate nucleotide
  modified_base 130..132: mod_base = OTHER; note = 2'-O-methyl-3'-phosphonoacetate nucleotide

SEQ ID NO: 53 - RNA, 133 nt, synthetic construct; identical to SEQ ID NO: 50
  source 1..133: mol_type = other RNA
  modified_base 1..3: mod_base = OTHER; note = 2'-O-methyl-3'-phosphorothioate nucleotide
  modified_base 131..132: mod_base = OTHER; note = 2'-O-methyl-3'-phosphonoacetate nucleotide

SEQ ID NO: 54 - RNA, 133 nt, synthetic construct; identical to SEQ ID NO: 50
  source 1..133: mol_type = other RNA
  modified_base 1..3: mod_base = OTHER; note = 2'-O-methyl-3'-phosphorothioate nucleotide
  modified_base 132: mod_base = OTHER; note = 2'-O-methyl-3'-phosphonoacetate nucleotide

(19) United States
(12) Patent Application Publication
(10) Pub. No.: US 2026/0152741 A1
(43) Pub. Date: Jun. 4, 2026

(54) METHOD FOR USING GUIDE RNAS WITH CHEMICAL MODIFICATIONS

(71) Applicant: Agilent Technologies, Inc., Santa Clara, CA (US)

(72) Inventors: Daniel E. RYAN, San Francisco, CA (US); Douglas J. Dellinger, Boulder, CO (US); Robert Kaiser, Santa Clara, CA (US)

(21) Appl. No.: 19/462,517

(22) Filed: Jan. 28, 2026

Related U.S. Application Data
(63) Continuation of application No. 17/945,060, filed on Sep. 14, 2022.
(60) Provisional application No. 63/243,985, filed on Sep. 14, 2021.
(60) Provisional application No. 63/339,737, filed on May 9, 2022.

(51) Int. Cl. C12N 15/11; C12N 9/22; C12N 15/90

(52) U.S. Cl. CPC C12N 15/111; C12N 9/22; C12N 15/907; C12N 2310/20; C12N 2310/312; C12N 2310/315; C12N 2310/321; C12N 2800/80

(57) ABSTRACT

The present invention provides compositions and methods for inducing CRISPR/Cas-based editing of a target nucleic acid (e.g., target DNA or target RNA), or modulating the expression of a target nucleic acid, in vitro or in a cell, using modified guide RNAs (gRNAs) that incorporate one or more chemically-modified nucleotides. In some aspects, these modified gRNAs provide superior performance under challenging conditions.

### CROSS-REFERENCE TO RELATED APPLICATIONS

The present application is a Continuation of U.S. patent application Ser. No. 17/945,060, filed Sep. 14, 2022, which claims the benefit of priority to U.S. Provisional Application Nos. 63/243,985, filed Sep. 14, 2021, and 63/339,737, filed May 9, 2022, the entire contents of each of which is incorporated herein by reference in its entirety.

### REFERENCE TO SEQUENCE LISTING

The instant application contains a Sequence Listing which has been submitted electronically in XML format and is hereby incorporated by reference in its entirety. Said XML copy, created on Jan. 22, 2026, is named 0528696.00059_20210117-11_SL.xml and is 118,871 bytes in size.

### TECHNICAL FIELD

The present disclosure relates to the field of molecular biology. In particular, the present disclosure relates to the clusters of regularly interspaced short palindromic repeats (CRISPR) technology.

### BACKGROUND

The native prokaryotic CRISPR-Cas system comprises an array of short repeats with intervening variable sequences of constant length (i.e., clusters of regularly interspaced short palindromic repeats, or “CRISPR”), and CRISPR-associated (“Cas”) proteins. The RNA of the transcribed CRISPR array is processed by a subset of the Cas proteins into small guide RNAs, which generally have two components as discussed below. There are at least six different systems: Type I, Type II, Type III, Type IV, Type V, and Type VI. The enzymes involved in the processing of the RNA into mature crRNA are different in these six systems. In native prokaryotic Type II systems, the guide RNA (“gRNA”) comprises two short, non-coding RNA segments referred to as CRISPR RNA (“crRNA”) and trans-acting RNA (“tracrRNA”). In native Type V systems, the guide RNA comprises a crRNA that is sufficient to form an active complex with a Cas12 (e.g., Cas12a is also known as Cpf1) protein without a tracrRNA segment. The gRNA forms a complex with a Cas protein (a ribonucleoprotein “RNP” complex). The gRNA:Cas protein complex binds a target polynucleotide sequence having a protospacer adjacent motif (“PAM”) and a protospacer, which comprises a sequence complementary to a portion of the gRNA. The recognition and binding of the target polynucleotide by the gRNA:Cas protein complex induces cleavage of the target polynucleotide. The native CRISPR-Cas system functions as an immune system in prokaryotes, where gRNA:Cas protein complexes recognize and silence exogenous genetic elements in a manner analogous to RNAi in eukaryotic organisms, thereby conferring resistance to exogenous genetic elements such as plasmids and phages.

Many enhancements and refinements of CRISPR technology have been and continue to be developed. Early approaches include using the CRISPR-Cas system to cleave both strands of a target DNA, and editing takes place by homologous recombination or non-homologous end joining due to the double-stranded break. Newer technologies include modulation of gene expression and other gene-editing methods. For instance, prime editing is a CRISPR-based technology for the editing of targeted sequences in DNA, and it allows for various forms of base substitutions, such as transversion and transition mutations. It also allows for precise insertions and deletions, including large deletions of up to about 700 bp long. Notably prime editing does not require an exogenous DNA repair template. Instead, a polymerization template containing the desired edits is included in the guide RNA, which complexes with a Cas protein that is fused with a polymerase (such as a reverse transcriptase). Upon binding a target site, the Cas protein nicks the target site, and the polymerase can synthesize a new strand of DNA using the polymerization template. Base editing is another gene-editing technique where a base editor enzyme, such as a cytidine deaminase, is delivered with a Cas protein and a guide RNA. The base editor enzyme is directed to the target site by the gRNA:Cas protein complex, and catalyzes deamination and hence mutation of cytidine residues at the target site. Modulation of gene expression may be achieved, for example, by fusing a transcriptional activator or inhibitor to a Cas protein that has no cleavage activity but can complex with a gRNA to bind to a target site. As a result, the transcriptional activator or inhibitor can regulate gene expression at the target site. The technique is thus called CRISPRa and CRISPRi, respectively, wherein “a” stands for activation and “i” stands for inhibition.

Despite these advances, there exists a need in the art for further improvements to CRISPR technology and, in particular, for improvements to the efficiency and stability of CRISPR-based systems, e.g., to bolster the adoption of CRISPR-based gene editing or modulation.

### DESCRIPTION OF THE FIGURES

FIG. 1 is a graph showing the results of a titration study in which an increasing amount of gRNA was mixed with a fixed amount of Cas9 protein for transfection into 0.2 million HepG2 cells where the HBB gene was targeted for creation of indels at the target site. These results illustrate the concept of a saturating amount of a transfected component for editing, where the increasing amount reaches a plateau of editing activity and further increases in the amount do not increase the editing yield in a constant number of cells.

FIG. 2 is a graph showing on- and off-target editing of HBB in HepG2 cells transfected with sub-saturating amounts of Cas mRNA and gRNA (0.0625 pmol of Cas9 mRNA and 10 pmol gRNA for 0.2 million cells) after washing the cells with PBS buffer to remove residual serum.

FIG. 3 is a graph showing on- and off-target editing of HBB in HepG2 cells transfected with sub-saturating amounts of Cas mRNA and gRNA (0.0625 pmol of Cas9 mRNA and 10 pmol gRNA for 0.2 million cells) after washing the cells with PBS buffer to remove residual serum.

FIG. 4 is a graph showing on- and off-target editing of HBB in HepG2 cells transfected with sub-saturating amounts of Cas mRNA and gRNA (0.5 pmol of Cas9 mRNA and 30 pmol gRNA for 0.2 million cells) when the cells were not washed with buffer to remove residual serum prior to transfection.

FIG. 5 is a graph showing on- and off-target editing of HBB in HepG2 cells transfected with sub-saturating amounts of Cas protein and gRNA (12.5 pmol of Cas9 protein and 30 pmol of sgRNA for 0.2 million cells) when the cells were not washed with buffer to remove serum prior to transfection.

FIG. 6 illustrates two exemplary gRNAs that incorporate 3×MS at the 5′ and 3′ end (top), or 3×MS at the 5′ end and 3×MP at the 3′ end (bottom).

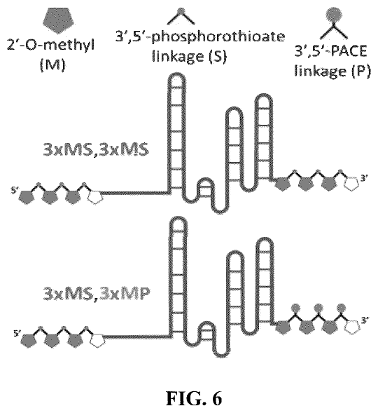

FIG. 7 is a graph showing the results of an experiment that evaluated the relative level of chemically-modified gRNA in K562 cells over time. Cells were transfected with gRNA in the absence of Cas protein, after washing the cells with PBS buffer to remove residual serum.

FIG. 8 is a graph showing on- and off-target editing of HBB in primary human T cells transfected with sub-saturating amounts of Cas9 mRNA and gRNA (0.0625 pmol of Cas9 mRNA and 5 pmol of sgRNA for 0.2 million cells) after washing the cells with PBS buffer to remove residual serum.

FIG. 9 is a graph showing the results of cytidine base editing of HBB in K562 cells using chemically-modified gRNA having MS or MP at the 3′ end relative to a control using unmodified gRNA. Cells were co-transfected with gRNA and an mRNA encoding a Cas9 nickase fused to a cytidine deaminase.

FIG. 10 is an illustration depicting prime editing using an exemplary CRISPR-Cas system.

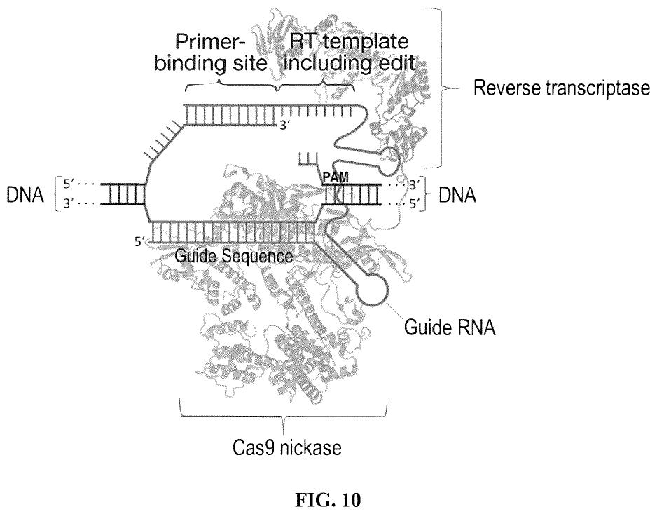

FIG. 11 is a graph showing the effectiveness of prime editing of EMX1 in K562 cells using an initial set of chemically-modified pegRNAs. Cells were co-transfected with pegRNA and mRNA encoding a Cas9 nickase fused to a reverse transcriptase.

FIG. 12 is a graph showing the effectiveness of prime editing of EMX1 in Jurkat cells using an initial set of chemically-modified pegRNAs. Cells were co-transfected with pegRNA and mRNA encoding a Cas9 nickase fused to a reverse transcriptase.

FIG. 13 is a graph showing the effectiveness of prime editing of EMX1 in K562 cells using a second set of chemically-modified pegRNAs. Cells were co-transfected with pegRNA and mRNA encoding a Cas9 nickase fused to a reverse transcriptase.

FIG. 14 is a graph showing the effectiveness of prime editing of EMX1 in Jurkat cells using a second set of chemically-modified pegRNAs. Cells were co-transfected with pegRNA and mRNA encoding a Cas9 nickase fused to a reverse transcriptase.

FIG. 15 is a graph showing the effectiveness of prime editing of RUNX1 in K562 cells using an initial set of chemically-modified pegRNAs. Cells were co-transfected with pegRNA and mRNA encoding a Cas9 nickase fused to a reverse transcriptase.

FIG. 16 is a graph showing the effectiveness of prime editing of RUNX1 in Jurkat cells using an initial set of chemically-modified pegRNAs. Cells were co-transfected with pegRNA and mRNA encoding a Cas9 nickase fused to a reverse transcriptase.

FIG. 17 illustrates the chemical structure of 2′-O-methyl-3′-phosphorothioate (MS) and 2′-O-methyl-3′-phosphonoacetate (MP), two examples of chemically-modified nucleotides that may be incorporated into the pegRNAs disclosed herein.

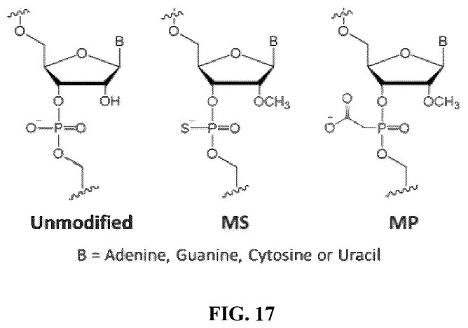

FIG. 18 illustrates prime editing of EMXI and RUNX1 using exemplary target sequences.

FIG. 19 is a graph showing the results of an experiment that assessed prime editing of EMX1 in K562 cells. In this case, the prime editing was used to knockout the PAM in EMX1. Cells were co-transfected with pegRNA and mRNA encoding a Cas9 nickase fused to a reverse transcriptase.

FIG. 20 is a graph showing the results of an experiment that assessed prime editing of EMX1 in Jurkat cells. In this case, the prime editing was used to knockout the PAM in EMX1. Cells were co-transfected with pegRNA and mRNA encoding a Cas9 nickase fused to a reverse transcriptase.

FIG. 21 is a graph showing the results of an experiment that assessed prime editing of RUNX1 in K562 cells. In this case, the prime editing was used to introduce a three-base insertion in RUNX1. Cells were co-transfected with pegRNA and mRNA encoding a Cas9 nickase fused to a reverse transcriptase.

FIG. 22 is a graph showing the results of an experiment that assessed prime editing of RUNX1 in Jurkat cells. In this case, the prime editing was used to introduce a three-base insertion in RUNX1. Cells were co-transfected with pegRNA and mRNA encoding a Cas9 nickase fused to a reverse transcriptase.

FIG. 23 is a graph showing the results of an experiment that assessed editing of the HBB sickle cell allele (and a known intergenic off-target locus) in unrinsed HepG2 cells co-transfected with sgRNA and mRNA encoding a Cas9 protein.

FIG. 24 is a graph showing the results of an experiment that assessed editing of the HBB sickle cell allele (and a known intergenic off-target locus) in unrinsed HepG2 cells transfected with ribonucleoprotein (RNP) complexes formed from chemically-modified sgRNA pre-complexed with Cas9 protein.

FIG. 25 is a graph showing the results of an experiment that assessed editing of the HBB sickle cell allele (and a known intergenic off-target locus) in unrinsed HepG2 cells transfected with ribonucleoprotein (RNP) complexes formed by chemically-modified 163 mer sgRNAs pre-complexed with Cas9 protein. The 163 mer sgRNAs were designed for CRISPRa SAM systems but were used with SpCas9 protein to produce indels instead of using them for gene activation by CRISPRa.

### DETAILED DESCRIPTION

Provided herein are methods for CRISPR/Cas-based genome editing and/or modulation of gene expression in a cell (e.g., a primary cell for use in ex vivo therapy) or an in vivo cell (e.g., a cell in an organ or tissue of a subject such as a human). In particular, the methods provided herein utilize chemically-modified guide RNAs (gRNAs) having enhanced activity or yield in gene editing or regulation compared to corresponding unmodified gRNAs. In some aspects, the present disclosure provides methods for editing a sequence of a target nucleic acid, or modulating expression of the target nucleic acid, in a cell by introducing a chemically-modified gRNA that hybridizes to the target nucleic acid together with either a Cas protein, an mRNA encoding a Cas protein, or a recombinant expression vector comprising a nucleotide sequence encoding a Cas protein. In some aspects, the Cas protein may be a variant that lacks nuclease activity (e.g., dCas9), or which possesses a nickase activity. In some aspects, the Cas protein is a fusion protein comprising a Cas polypeptide and a reverse transcriptase polypeptide. In some aspects, the present disclosure provides methods for preventing or treating a genetic disease in a subject by administering a sufficient amount of the chemically modified gRNA to correct a genetic mutation associated with the disease (e.g., by editing the genomic DNA of a patient or by modulating expression of a gene associated with the disease).

Aspects of the present disclosure employ conventional techniques of immunology, biochemistry, chemistry, molecular biology, microbiology, cell biology, genomics and recombinant DNA, which are within the skill of the art. See Sambrook, Fritsch and Maniatis, Molecular Cloning: A Laboratory Manual, 2nd edition (1989), Current Protocols in Molecular Biology (F. M. Ausubel, et al. eds., (1987)), the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)).

Oligonucleotides that are not commercially available can be chemically synthesized, e.g., according to the solid phase phosphoramidite triester method first described by Beaucage and Caruthers. Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### Definitions and Abbreviations

Unless specifically indicated otherwise, all technical and scientific terms used herein have the same meaning as commonly understood by those of ordinary skill in the art. In addition, any method or material similar or equivalent to a method or material described herein can be used in the practice of the methods and preparation of the compositions described herein. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “CRISPR-associated protein” or “Cas protein” or “Cas polypeptide” refers to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the “Cas mutant” or “Cas variant” substantially retains the nuclease activity of the Cas protein. In certain embodiments, the “Cas mutant” or “Cas variant” is mutated such that one or both nuclease domains are inactive (this protein may be referred to as a Cas nickase or dead Cas protein, respectively). In certain embodiments, the “Cas mutant” or “Cas variant” has nuclease activity. In certain embodiments, the “Cas mutant” or “Cas variant” lacks some or all of the nuclease activity of its wild-type counterpart. The term “CRISPR-associated protein” or “Cas protein” also includes a wild type Cpf1 protein, also referred to as Cas12a, of various species of prokaryotes (and named for Clustered Regularly Interspaced Short Palindromic Repeats from Prevotella and Francisella 1 ribonucleoproteins or CRISPR/Cpf1 ribonucleoproteins), a fragment thereof, or a mutant or variant thereof. Cas protein includes any of the CRISPR-associated proteins, including but not limited to any one in the six different CRISPR systems: Type I, Type II, Type III, Type IV, Type V, and Type VI.

The term “nuclease domain” of a Cas protein refers to the polypeptide sequence or domain within the protein which possesses the catalytic activity for DNA cleavage. Cas9 typically catalyzes a double-stranded break upstream of the PAM sequence. A nuclease domain can be contained in a single polypeptide chain, or cleavage activity can result from the association of two (or more) polypeptides. A single nuclease domain may consist of more than one isolated stretch of amino acids within a given polypeptide. Examples of these domains include RuvC-like motifs (amino acids 7-22, 759-766 and 982-989 in SEQ ID NO: 1) and HNH motifs (amino acids 837-863); see Gasiunas et al. (2012) Proc. Natl. Acad. Sci. USA 109:39, E2579-E2586 and WO/2013176772.

A synthetic guide RNA (“gRNA”) that has “gRNA functionality” is one that has one or more of the functions of naturally occurring guide RNA, such as associating with a Cas protein to form a ribonucleoprotein (RNP) complex, or a function performed by the guide RNA in association with a Cas protein (i.e., a function of the RNP complex). In certain embodiments, the functionality includes binding a target polynucleotide. In certain embodiments, the functionality includes targeting a Cas protein or a gRNA:Cas protein complex to a target polynucleotide. In certain embodiments, the functionality includes nicking a target polynucleotide. In certain embodiments, the functionality includes cleaving a target polynucleotide. In certain embodiments, the functionality includes associating with or binding to a Cas protein. For example, the Cas protein may be engineered to be a “dead” Cas protein (dCas) fused to one or more proteins or portions thereof, such as a transcription factor enhancer or repressor, a deaminase protein, a reverse transcriptase, a polymerase, etc., such that the fused protein(s) or portion(s) thereof can exert its functions at the target site. In certain embodiments, the functionality comprises base editing functionality. In other embodiments, the functionality includes prime editing functionality. In certain embodiments, the functionality includes activation, repression or interference of gene expression. In other embodiments, the functionality includes epigenetic modifications. In certain embodiments, the functionality is any other known function of a guide RNA in a CRISPR-Cas system with a Cas protein, including an artificial CRISPR-Cas system with an engineered Cas protein. In certain embodiments, the functionality is any other function of natural guide RNA. The synthetic guide RNA may have gRNA functionality to a greater or lesser extent than a naturally occurring guide RNA. In certain embodiments, a synthetic guide RNA may have greater activities as to one function and lesser activities as to another function in comparison to a similar naturally occurring guide RNA.

A Cas protein having a single-strand “nicking” activity refers to a Cas protein, including a Cas mutant or Cas variant, that has reduced ability to cleave one of two strands of a dsDNA as compared to a wild type Cas protein. For example, in certain embodiments, a Cas protein having a single-strand nicking activity has a mutation (e.g., amino acid substitution) that reduces the function of the RuvC domain (or the HNH domain) and as a result reduces the ability to cleave one strand of the target DNA. Examples of such variants include the D10A, H839A/H840A, and/or N863A substitutions in S. pyogenes Cas9, and also include the same or similar substitutions at equivalent sites in Cas9 enzymes of other species.

A Cas protein having “binding” activity or that “binds” a target polynucleotide refers to a Cas protein which forms a complex with a guide RNA and, when in such a complex, the guide RNA hybridizes with another polynucleotide, such as a target polynucleotide sequence, via hydrogen bonding between the bases of the guide RNA and the other polynucleotide to form base pairs. The hydrogen bonding may occur by Watson-Crick base pairing or in any other sequence specific manner. The hybrid may comprise two strands forming a duplex, three or more strands forming a multi-stranded triplex, or any combination of these.

A “CRISPR system” is a system that utilizes at least one Cas protein and at least one gRNA to provide a function or effect, including but not limited to gene editing, DNA cleavage, DNA nicking, DNA binding, regulation of gene expression, CRISPR activation (CRISPRa), CRISPR interference (CRISPRi), and any other function that can be achieved by linking a Cas protein to another effector, thereby achieving the effector function on a target sequence recognized by the Cas protein. For example, a nuclease-free Cas protein can be fused to a transcription factor, a deaminase, a methylase, a reverse transcriptase, etc. The resulting fusion protein, in the presence of a guide RNA for the target, can be used to edit, regulate the transcription of, deaminate, or methylate, the target. As another example, in prime editing, a Cas protein is used with a reverse transcriptase or other polymerases (optionally as a fusion protein) to edit target nucleic acids in the presence of a pegRNA.

A “fusion protein” is a protein comprising at least two peptide sequences (i.e., amino acid sequences) covalently linked to each other, where the two peptide sequences are not covalently linked in nature. The two peptide sequences can be linked directly (with a bond in between) or indirectly (with a linker in between, wherein the linker may comprise any chemical structure, including but not limited to a third peptide sequence).

A “prime editor” is a molecule, or a collection of multiple molecules, that has both Cas protein and reverse transcriptase activities. In some embodiments, the Cas protein is a nickase. In some embodiments, the prime editor is a fusion protein comprising both a Cas protein and a reverse transcriptase. As indicated elsewhere in this disclosure, other polymerases can be used in prime editing in lieu of a reverse transcriptase, so a prime editor may comprise a polymerase that is not a reverse transcriptase, in lieu of the RT. Different versions of prime editor have been developed and are referred to as PE1, PE2, PE3, etc. For example “PE2” refers to a PE complex comprising a fusion protein (PE2 protein) comprising a Cas9(H840A) nickase and a variant of MMLV RT having the following structure: [NLS]-[Cas9(H840A)-[linker]-[MMLV_RT(D200N)(T330P)(L603W)(T306K)(W313F)], and a desired pegRNA. “PE3” refers to PE2 plus a second-strand nicking guide RNA that complexes with the PE2 protein and introduces a nick in the non-edited DNA strand in order to stimulate the cell into repairing the target region, which facilitates incorporation of the edits into the genome (see Anzalone et al. 2019; see Liu WO2020191153). Prime editors use specialized gRNAs, referred to as prime editing gRNAs or “pegRNAs”, as described in detail elsewhere in this disclosure.

A “base editor” or “BE” is a molecule, or a collection of multiple molecules, that has both Cas protein (or mutated protein) and deaminase or transglycosylation activities. Base editors (BEs) are typically fusions of a Cas domain and a nucleotide modification domain (e.g., a natural or evolved deaminase, such as a cytidine deaminase, e.g., APOBEC1 (“apolipoprotein B mRNA editing enzyme, catalytic polypeptide 1”), CDA (“cytidine deaminase”), and AID (“activation-induced cytidine deaminase”) or adenosine deaminase, e.g., TadA (Bacterial tRNA-specific adenosine deaminase)).Two classes of deaminase base editors have been generally described to date: cytosine base editors (“CBE”) that convert target C:G base pairs to T:A base pairs, and adenosine base editors (“ABE”) which convert A:T base pairs to G:C base pairs. Collectively, these two classes of base editors enable the targeted installation of all possible transition mutations (C-to-T, G-to-A, A-to-G, T-to-C, C- to-U, and A-to-U), see Gaudelli, N. M. et al, Programmable base editing of A:T to G:C in genomic DNA without DNA cleavage. Nature 551, 464-471 (2017), which is incorporated herein by reference. Another nucleotide modification domain used in base editing is a transglycosylase domain such as a wild-type tRNA guanine transglycosylase (TGT), or a variant thereof, e.g., a TGT that substitutes a first nucleobase (i.e., a thymine) for a second nucleobase at a ribose-nucleobase glycosidic bond. The transglycosylase editor provides for thymine-to-guanine or “TGBE” (or adenine-to-cytosine or “ACBE”) transversion base editors. In some cases, base editors may also include proteins or domains that alter cellular DNA repair processes to increase the efficiency and/or stability of the resulting single-nucleotide change. In some embodiments the base editors comprise one or more NLSs (Nuclear Localization Sequence), and may further include one or more Uracil-DNA glycosylase inhibitor (UGI) domains, which are capable of inhibiting Uracil-DNA glycosylase, thereby improving base editing efficiency of C to T base editor proteins. In some embodiments, the Cas domain is a nickase (e.g. nCas9). In some embodiments, the Cas protein is a fully nuclease-inactivated protein or a dead Cas9 “dCas9”. In some embodiments, the base editor is a fusion protein comprising both a Cas protein (or portion thereof) and a deaminase (or portion thereof). In some embodiments, the base editor is a fusion protein comprising both a Cas protein (or portion thereof) and a transglycosylase (or portion thereof). Different versions of base editors that represent improvements over prior systems have been developed such as base editors with different or expanded PAM compatibilities (see: Kim, Y. B. et al. Increasing the genome-targeting scope and precision of base editing with engineered Cas9-cytidine deaminase fusions. Nature biotechnology 35, 371-376 (2017); Hu, J. H. et al. Evolved Cas9 variants with broad PAM compatibility and high DNA specificity. Nature 556, 57-63 (2018); Li, X. et al. Base editing with a Cpf1-cytidine deaminase fusion. Nature biotechnology 36, 324-327 (2018)), High fidelity base editors with reduced off-target activity (see: Hu, J. H. et al. Evolved Cas9 variants with broad PAM compatibility and high DNA specificity. Nature 556, 57-63 (2018); Rees, H. A. et al. Improving the DNA specificity and applicability of base editing through protein engineering and protein delivery. Nat Commun 8, 15790 (2017); Kleinstiver, B. P., Pattanayak, V., Prew, M. S. & Nature, T.-S. Q. High-fidelity CRISPR-Cas9 nucleases with no detectable genome-wide off-target effects. Nature (2016); Chen, J. S. et al. Enhanced proofreading governs CRISPR-Cas9 targeting accuracy. Nature 550, 407-410 (2017); Slaymaker, I. M. et al. Rationally engineered Cas9 nucleases with improved specificity. Science 351, 84-88 (2016)), base editors with narrower editing windows (normally ˜5 nucleotides wide) (see: Kim, Y. B. et al. Increasing the genome-targeting scope and precision of base editing with engineered Cas9-cytidine deaminase fusions. Nature biotechnology 35, 371-376 (2017).), and a cytidine base editor (BE4) with reduced by-products (see: Komor, A. C. et al. Improved base excision repair inhibition and bacteriophage Mu Gam protein yields C:G-to-T:A base editors with higher efficiency and product purity. Sci Adv 3, eaao4774 (2017)). The different versions of base editors are referred to as BE1, BE2, BE3, BE4 etc. Unlike prime editors, base editors function in concert with “conventional” gRNAs (e.g. Cas9 style or Cpf1 style), that program the Cas effector portion of the base editor to target the nucleic acid at a desired sequence location.

A “guide RNA” (or “gRNA”) generally refers to an RNA molecule (or a group of RNA molecules collectively) that can bind to a Cas protein and aid in targeting the Cas protein to a specific location within a target polynucleotide (e.g. a DNA). Thus, a guide RNA comprises a guide sequence that can hybridize to a target sequence, and another part of the guide RNA (the “scaffold”) functions to bind a Cas protein to form a ribonucleoprotein (RNP) complex of the guide RNA and the Cas protein. There are various styles of guide RNAs, including but not limited to the Cas9 style and the Cpf1 style of guide RNAs. A “Cas9 style” of guide RNA comprises a crRNA segment and a tracrRNA segment. As used herein, the term “crRNA” or “crRNA segment” refers to an RNA molecule or portion thereof that includes a polynucleotide-targeting guide sequence; a scaffold sequence which helps to interact with a Cas protein; and, optionally, a 5′-overhang sequence. As used herein, the term “tracrRNA” or “tracrRNA segment” refers to an RNA molecule or portion thereof that includes a protein-binding segment capable of interacting with a CRISPR-associated protein, such as a Cas9. In addition to Cas9, there are other Cas proteins employing the Cas9 style of guide RNAs, and the word “Cas9” is used in the term “Cas9 style” merely to specify a representative member of the various Cas proteins that employ this style. A “Cpf1 style” is a one-molecule guide RNA comprising a scaffold that is 5′ to a guide sequence. In the literature, the Cpf1 guide RNA is often described as having only a crRNA but not a tracrRNA. It should be noted that, regardless of the terminology, all guide RNAs have a guide sequence to bind to the target, and a scaffold region that can interact with a Cas protein. Unlike prime editing which uses specialized gRNA (pegRNA), base editing uses conventional gRNAs (i.e. Cas9 style and Cpf1 style).

The term “guide RNA” encompasses a single-guide RNA (“sgRNA”) that contains all functional parts in one molecule. For example, in a sgRNA of the Cas9 style, the crRNA segment and the tracrRNA segment are located in the same RNA molecule. As another example, the Cpf1 guide RNA is naturally a single-guide RNA molecule. The term “guide RNA” also encompasses, collectively, a group of two or more RNA molecules; for example, the crRNA segment and the tracrRNA segment may be located in separate RNA molecules. Furthermore, the term “gRNA” as used herein encompasses guide RNAs that are used in prime editing (pegRNA), base editing and gene expression modulation and any other CRISPR technology that employs gRNAs.

Optionally, a “guide RNA” may comprise one or more additional segments that serve one or more accessory functions upon being recognized and bound by cognate polypeptides or enzymes that perform molecular functions alongside the function of the Cas protein associated with the gRNA. For example, a gRNA for prime editing (which is commonly referred to as a “pegRNA”) may comprise a primer binding site and a reverse transcriptase template. In another example, the gRNA may comprise one or more polynucleotide segments that form one or more aptamers (e.g. MS2 aptamer) that recognize and bind aptamer-binding polypeptides (optionally fused to other polypeptides, (e.g. MS2-p65-HSF1) that serve accessory functions such as transcriptional activation alongside the Cas protein or Cas fusion protein (e.g. dCas9-VP64), these systems are known as a synergistic activation mediator “SAM” system; see S. Konermann et al., Genome-scale transcriptional activation by an engineered CRISPR-Cas9 complex. Nature. 517, 583-588 (2015)., see M. A. Horlbeck et al., Compact and highly active next-generation libraries for CRISPR-mediated gene repression and activation. eLife. 5, e19760 (2016)).

Optionally, a “guide RNA” may comprise an additional polynucleotide segment (such as a 3′ (or 5′)-terminal polyuridine tail, a hairpin, a stem loop, a toeloop etc.) that can increase the stability of the gRNA by impeding its degradation, as can occur for example by nucleases such as endonucleases and/or exonucleases.

The term “guide sequence” refers to a contiguous sequence of nucleotides in a gRNA (or pegRNA) which has partial or complete complementarity to a target sequence in a target polynucleotide and can hybridize to the target sequence by base pairing facilitated by a Cas protein.

In some cases, a target sequence is adjacent to a PAM site (the PAM sequence). In some cases, the target sequence may be located immediately upstream of the PAM sequence. A target sequence, which hybridizes to the guide sequence, may be immediately downstream from the complement of the PAM sequence. In other examples such as Cpf1, the location of the target sequence, which hybridizes to the guide sequence, may be upstream from the complement of the PAM sequence.

A guide sequence can be as short as about 14 nucleotides and as long as about 30 nucleotides. Typical guide sequences are 15, 16, 17, 18, 19, 20, 21, 22, 23 and 24 nucleotides long. The length of the guide sequence varies across the two classes and six types of CRISPR-Cas systems mentioned above. Synthetic guide sequences for Cas9 are usually 20 nucleotides long, but can be longer or shorter. When a guide sequence is shorter than 20 nucleotides, it is typically a deletion from the 5′-end compared to a 20-nucleotide guide sequence. By way of example, a guide sequence may consist of 20 nucleotides complementary to a target sequence. In other words, the guide sequence is identical to the 20 nucleotides upstream of the PAM sequence, except the A/U difference between DNA and RNA. If this guide sequence is truncated by 3 nucleotides from the 5′-end, nucleotide 4 of the 20-nucleotide guide sequence now becomes nucleotide 1 in the 17-mer, nucleotide 5 of the 20-nucleotide guide sequence now becomes nucleotide 2 in the 17-mer, etc. The new position is the original position minus 3 for a 17-mer guide sequence.

As used herein, the term “prime editing guide RNA” (or “pegRNA”) refers to a guide RNA (gRNA) that comprises a reverse transcriptase template sequence encoding one or more edits to a target sequence of a nucleic acid, and a primer binding site that can bind to a sequence in the target region (also called a target site). For example, a pegRNA may comprise a reverse transcriptase template sequence comprising one or more nucleotide substitutions, insertions or deletions to a sequence in the target region. A pegRNA has the function of complexing with a Cas protein and hybridizing to a target sequence in a target region, usually in the genome of a cell, to result in editing of a sequence in the target region. In some embodiments, without being limited to a theory, the pegRNA forms an RNP complex with a Cas protein and binds the target sequence in the target region, the Cas protein makes a nick on one strand of the target region to result in a flap, the primer binding site of the pegRNA hybridizes with the flap, the reverse transcriptase uses the flap as a primer on the hybridized reverse transcriptase template of the pegRNA which serves as a template to synthesize a new DNA sequence onto the nicked end of the flap which then contains the desired edits, and ultimately, this new DNA sequence replaces an original sequence in the target region, resulting in editing of the target.

A “pegRNA” may comprise the reverse transcriptase template and primer binding site near its 5′ end or 3′ end. The “prime editing end” is one end of the pegRNA, either 5′ or 3′, that is closer to the reverse transcriptase template and primer binding site than to the guide sequence. The other end of the pegRNA is the “distal end”, which is closer to the guide sequence than to the reverse transcriptase template or primer binding site. Thus, the order of these components is, in either 5′ or 3′ orientation:
prime editing end—(primer binding site and reverse transcriptase template)—(guide sequence and scaffold)—distal end
   where the parentheses indicate that the two segments mentioned within could be switched in order with respect to each other, depending on the style of the pegRNA (e.g. Cas9 style or Cpf1 style) as well as the position of the prime editing end (i.e., a 5′ end or a 3′ end). It should be noted that if the pegRNA is not a single-guide RNA but comprises more than one RNA molecule, the prime editing end refers to the end closer to the primer binding site and reverse transcriptase template in the RNA molecule containing these components, whereas the opposite end of this RNA molecule is the distal end. The guide sequence may be in a different RNA molecule of the pegRNA, distinct from the RNA molecule bearing the prime editing end and the distal end.

A “nicking guide RNA” or “nicking gRNA” is a guide RNA (not a pegRNA) that can be optionally added in prime editing to cause nicking of the strand that is not being edited, in or near the target region. Such nicking helps to stimulate the cell in which prime editing is taking place to repair the relevant area, i.e. the target region.

An “extension tail” is a stretch of nucleotides of 1, 2, 3 4, 5, 6, 7, 8, 9, or 10 nucleotides that can be added to either the 5′ end or 3′ end of a guide RNA, such as a pegRNA. A “poly(N) tail” is a homopolymer extension tail, containing 1-10 nucleotides with the same nucleobase, for example A, U, C or T. A “polyuridine tail” or “polyU tail” is a poly(N) tail containing 1-10 uridines. Similarly, a “polyA tail” contains 1-10 adenosines.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “nucleotide analog” or “modified nucleotide” refers to a nucleotide that contains one or more chemical modifications (e.g., substitutions), in or on the nitrogenous base of the nucleoside (e.g., cytosine (C), thymine (T) or uracil (U), adenine (A) or guanine (G)), in or on the sugar moiety of the nucleoside (e.g., ribose, deoxyribose, modified ribose, modified deoxyribose, six-membered sugar analog, or open-chain sugar analog), or the phosphate.

The term “gene” or “nucleotide sequence encoding a polypeptide” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “nucleic acid”, “polynucleotide” or “oligonucleotide” refers to a DNA molecule, an RNA molecule, or analogs thereof. As used herein, the terms “nucleic acid”, “polynucleotide” and “oligonucleotide” include, but are not limited to DNA molecules such as cDNA, genomic DNA or synthetic DNA and RNA molecules such as a guide RNA, messenger RNA or synthetic RNA. Moreover, as used herein, the terms include single-stranded and double-stranded forms.

The term “hybridization” or “hybridizing” refers to a process where completely or partially complementary polynucleotide strands come together under suitable hybridization conditions to form a double-stranded structure or a region in which the two constituent strands are joined by hydrogen bonds. As used herein, the term “partial hybridization” includes where the double-stranded structure or region contains one or more bulges or mismatches. Although hydrogen bonds typically form between adenine and thymine or adenine and uracil (A and T, or A and U, respectively) or cytosine and guanine (C and G), other non-canonical base pairs may form (see, e.g., Adams et al., “The Biochemistry of the Nucleic Acids,” 11th ed., 1992). It is contemplated that modified nucleotides may form hydrogen bonds that allow or promote hybridization in a non-canonical way.

The term “complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, the term “portion”, “segment”, “element”, or “fragment” of a sequence refers to any portion of the sequence (e.g., a nucleotide subsequence or an amino acid subsequence) that is smaller than the complete sequence. Portions, segments, elements, or fragments of polynucleotides can be of any length that is more than 1, for example, at least 5, 10, 15, 20, 25, 30, 40, 50, 75, 100, 150, 200, 300 or 500 or more nucleotides in length.

The term “oligonucleotide” as used herein denotes a multimer of nucleotides. For example, an oligonucleotide may have about 2 to about 200 nucleotides, up to about 50 nucleotides, up to about 100 nucleotides, up to about 500 nucleotides in length, or any integer value between 2 and 500 in nucleotide number. In some embodiments, an oligonucleotide may be in the range of 30 to 300 nucleotides in length or 30 to 400 nucleotides in length. Oligonucleotides may contain ribonucleotide monomers (i.e., may be oligoribonucleotides) and/or deoxyribonucleotide monomers. An oligonucleotide may be 10 to 20, 21 to 30, 31 to 40, 41 to 50, 51-60, 61 to 70, 71 to 80, 80 to 100, 100 to 150, 150 to 200, 200 to 250, 250 to 300, 300 to 350, or 350 to 400 nucleotides in length, for example, and any integer value in between these ranges.

A “recombinant expression vector” is a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators), as well as those that confer certain binding affinity or antigenicity to the recombinant protein produced from the expression vector.

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

“Editing” a nucleic acid target means causing a change in the nucleotide sequence of the target. The change may be an insertion, deletion or substitution, each of a single nucleotide or multiple nucleotides. Where multiple nucleotides are inserted, deleted or substituted, the nucleotides may be consecutive or not consecutive. The change may be a combination of any of the above. “Editing” comprises “base editing” and “prime editing” technologies.

“Editing efficiency” is a measure of the Cas-induced editing achieved in one or more cells. The results of genome editing at the target, and potential off-target sites, can be measured using standard methods known in the art, for example, genomic DNA sequencing, RNA sequencing, or deep sequencing of PCR amplicons of the target site and any off-target sites of interest. Also, indel mutations in genomic DNA can be identified by using the SURVEYOR® mutation detection kit (Integrated DNA Technologies, Coralville, Iowa) or the Guide-it™ Indel Identification Kit (Clontech, Mountain View, CA). In addition, techniques that measure the presence or absence of proteins, e.g. gel or capillary electrophoresis, Western blotting, flow cytometry, or mass spectrometry techniques can be used to quantify the efficiency of editing aimed to introduce or knock-out protein-coding genes. These techniques can be applied to populations of cells in bulk preparations or at a single cell level. In some embodiments, the efficiency is measured using the number of the correct edits in a population of cells measured in hulk or at a single-cell level. In some embodiments, the efficiency is measured as the percentage of the targets that are correctly edited, or the number or percentage of the cells that show the corrected genotype or phenotype.

“Modulating the expression of a gene” means altering (decreasing or activating) the expression of a specific gene product. CRISPR activation or “CRISPRa” refers to the activation of a gene whereas CRISPR interference or “CRISPRi” refers to the interference of a gene expression. Both systems use a nuclease deficient Cas protein (dCas9) fused or interacting in combination with transcriptional effector(s) (activator or repressor). CRISPRa may be performed in a SAM system (dCas9-VP64) as described previously. When used with gene-specific CRISPRa, the gRNA comprising a MS2 aptamer recruits the MS2-p65-HSF1 fusion to the transcriptional start site (TSS) of the targeted gene to initiate activation. CRISPRa and CRISPRi can be both performed and combined in a multiplexed fashion (e.g., targeting of multiple genes). CRISPRoff is a programmable epigenetic memory writer consisting of a dead Cas9 fusion protein that establishes DNA methylation and repressive histone modifications that can heritably alter gene expression (Nunez et al., Genome-wide programmable transcriptional memory by CRISPR-based epigenome editing, Cell. (2021) 184(9):2503-2519.

“Gene expression modulation efficiency” can be measured for example by techniques that measure the relative or absolute levels of different RNAs, e.g. qRT-PCR or RNA-sequencing, or by various methods that measure the relative or absolute levels of proteins, e.g. gel or capillary electrophoresis, Western blotting, flow cytometry, or mass spectrometry techniques. These techniques can be applied to populations of cells in bulk preparations or at a single cell level. In some embodiments, the efficiency is measured using the amount of the protein or RNA expressed. from the target gene in a population of cells measured in bulk or at a single-cell level.

The term “single nucleotide polymorphism” or “SNP” refers to a change of a single nucleotide with a polynucleotide, including within an allele. This can include the replacement of one nucleotide by another, as well as deletion or insertion of a single nucleotide. Most typically, SNPs are biallelic markers although tri- and tetra-allelic markers can also exist. By way of non-limiting example, a nucleic acid molecule comprising SNP A\C may include a C or A at the polymorphic position.

“Nucleases” as used herein means enzymes capable of cleaving the phosphodiester linkage between nucleotides of nucleic acids. Nucleases variously can effect both single and/or double stranded cleavage of DNA and/or RNA molecules. In living organisms, they are essential machinery for many aspects of DNA repair. As used herein, nucleases refer to both exonucleases and endonucleases and encompass ribonucleases as well as deoxyribonucleases.

The term “primary cell” refers to a cell isolated directly from a multicellular organism. Primary cells typically have undergone very few population doublings and are therefore more representative of the main functional component of the tissue from which they are derived in comparison to continuous (tumor or artificially immortalized) cell lines. In some cases, primary cells are cells that have been isolated and then used immediately. In other cases, primary cells cannot divide indefinitely and thus cannot be cultured for long periods of time in vitro.

The terms “nucleases-containing fluid” is used herein to refer to any medium in which nucleases are present. For instance, the medium can be a cell culture medium or a medium that originated from a cell culture medium, meaning that the cells were transferred from a cell culture medium, into a new medium with or without washing the cells but without removing all the components contained in the original medium, and therefore may still contain nucleases. For instance, a cell may be transferred from a cell culture medium to a reaction medium without washing the cell or without removal of substantially all the components of the cell culture medium, and therefore nucleases may be present at the time of contacting the cell with the gRNA and the Cas protein (RNP) or the gRNA and the mRNA or DNA vector encoding the editing Cas effector. The fluid may be a serum, a human serum, an animal serum, a bovine serum (BSA), a fetal serum, a cerebrospinal fluid (CSF) or another bodily fluid.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., primary cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject,” “patient,” and “individual” are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent (e.g., Cas protein, modified gRNA/pegRNA, etc.) that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other agents, timing of administration, and the physical delivery system in which it is carried.

As disclosed herein, a number of ranges of values are provided. It is understood that each intervening value between the upper and lower limits of that range is also specifically contemplated. Each smaller range or intervening value encompassed by a stated range is also specifically contemplated. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

Several chemically-modified nucleotides are described herein. Note that each of MS, MP, and MSP can mean the corresponding modification, or a nucleotide comprising the corresponding modification. The following abbreviations shall be used in relevant contexts:
“PACE”: phosphonoacetate
“MS”: 2′-O-methyl-3′-phosphorothioate
“MP”: 2′-O-methyl-3′-phosphonoacetate
“MSP”: 2′-O-methyl-3′-thiophosphonoacetate
“2′-MOE”: 2′-O-methoxyethyl

Other definitions of terms may appear throughout the specification.

The present invention demonstrates that certain modifications of a guide RNA, in specific positions, render the guide RNA extra resistant to degradation by nucleases. This is particularly important for in vivo delivery of guide RNAs for CRISPR-mediated gene editing or modulation of gene expression, as nuclease activities are high in vivo. For example, bodily fluids, such as serum and cerebrospinal fluid (CSF), contain relatively abundant nucleases. In such a challenging environment, the guide RNA tends to be degraded, thus its concentration does not reach a level that can achieve higher performance (i.e., sub-saturated). Therefore, any increase in guide RNA concentration, and thereby in the chance of gene editing and modulation of gene expression, would be significant in this industry. This invention provides the surprising discovery that certain guide RNAs, for example those with phosphorothioate modifications at the 5′ end as well as phosphonocarboxylate or thiophosphonocarboxylate modifications at the 3′ end, led to higher CRISPR activities even in the presence of serum, as compared to counterparts that are unmodified or contains other modifications (e.g. phosphorothioate in the place of phosphonocarboxylate or thiophosphonocarboxylate but otherwise the same).

Similarly, cells to be subjected to CRISPR-mediated editing/modulation for ex vivo therapy are usually in cell culture media that contain serum, or, if freshly harvested from a subject, contain bodily fluids in the environment. As such, nucleases present in the serum or bodily fluids would degrade the guide RNAs delivered to the cells and reduce the efficiency of CRISPR-mediated editing/modulation. Although cells can be washed before CRISPR treatment in order to lower the amount of serum or bodily fluid, extensive washing may be unhealthy to the cells. Furthermore, CRISPR-mediated editing/modulation does not happen immediately after the guide RNA and other CRISPR effectors are added to the cells, and the cells need to be cultured for a period of time. Culturing in the absence of serum is often detrimental to cells, and is a risk factor for ex vivo therapy since the cells will be delivered to a patient later. The modified guide RNAs of the present invention, which are more resistant to nuclease degradation, is a significant improvement for resolving these problems.

Modified guide RNAs of the present invention are useful when introduced into cells in a “naked” manner and directly exposed to nucleases, e.g., co-transfected or otherwise delivered with a DNA or mRNA encoding a Cas protein. However, even when the guide RNA is not naked, for example present in a ribonucleoprotein (RNP) with the Cas protein, or in a nanoparticle with or without the Cas protein, the modifications described herein are also advantageous.

In one aspect of the present disclosure, methods are provided for editing a target region, or modulating expression of a target gene in a target region, in a nucleic acid in a cell. The methods comprise providing to the cell a) a CRISPR-associated (“Cas”) protein, and b) a modified guide RNA comprising a 5′ end and a 3′ end, a guide sequence that is capable of hybridizing to the target sequence in the target region, and a scaffold region that interacts with the Cas protein. The modified guide RNA also comprises one or more phosphorothioate modifications within 5 nucleotides of the 5′ end, and at least two consecutive phosphonocarboxylate or thiophosphonocarboxylate modifications within 5 nucleotides of the 3′ end. The cell exists ex vivo in the presence of nuclease containing fluids, or exists in vivo. In the present methods, providing the Cas protein and the modified guide RNA to the cell results in editing of the target region or modulation of expression of the target gene.

In some embodiments, the modified guide RNA comprises 2, 3, 4, or 5 phosphonocarboxylate or thiophosphonocarboxylate modifications within 5 nucleotides of the 3′ end. The at least two consecutive phosphonocarboxylate or thiophosphonocarboxylate modifications within 5 nucleotides of the 3′ end of the gRNA may comprise at least 2, 3, 4, or 5 MP nucleotides, which may be arranged in any order, including two consecutive modified nucleotides and one or two nonconsecutive modified nucleotides, three consecutive modified nucleotides and one nonconsecutive modified nucleotides, two pairs of two consecutive modified nucleotides, or five two consecutive modified nucleotides. In some embodiments, the modified guide RNA comprises at least two, at least three, at least four, or five consecutive MP nucleotides within 5 nucleotides of the 3′ end. In some embodiments, the modified guide RNA comprises 1, 2, 3, 4, or 5 phosphorothioate modifications within 5 nucleotides of the 5′ end. The one or more phosphorothioate modifications within 5 nucleotides of the 5′ end of the gRNA may comprise at least 1, at least 2, at least 3, at least 4, or 5 MS nucleotides, which may be arranged in any order, including consecutively or nonconsecutively. In some embodiments of the present methods, the modified guide RNA comprises at least two, at least three, at least four, or five consecutive MS nucleotides within 5 nucleotides of the 5′ end. The one or more modified nucleotides within 5 nucleotides of the 3′ or 5′ end of the gRNA may be independently selected (e.g., the number and/or order of modified nucleotides may be different on the 5′ and the 3′ end of the gRNA).

In some embodiments, the modified guide RNA further comprises modified nucleotide(s) located outside of 5 nucleotides within the 5′ end and 3′ end. The modified guide RNA may comprise one or more modifications in the guide sequence that enhance target specificity (as described, e.g., in U.S. Pat. No. 10,767,175). For example, the modified guide RNA may comprise a modified nucleotide at position 5 or position 11 of the modified guide sequence.

In some embodiments, the modified guide RNA is a single guide RNA. In some embodiments, the guide RNA is a single-guide RNA comprising exactly or at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 nucleotides, and/or up to 180, 179, 178, 177, 176, 175, 174, 173, 172, 171, 170, 169, 168, 167, 166, 165, 164, 163, 162, 161, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, or 120 nucleotides. It is expressly contemplated that any of the foregoing minima and maxima can be combined to form a range, as long as the minimum as less than the maximum.

In some embodiments, the Cas protein is provided to the cell as an mRNA encoding a Cas protein or a variant or fusion protein thereof. In some embodiments, the Cas protein is provided to the cell as a recombinant expression vector comprising a nucleotide sequence encoding a Cas protein or a variant or fusion protein thereof. The cell can be transfected with the mRNA or expression vector encoding the Cas protein, separately or together with the modified guide RNA. In some embodiments, the cell is co-transfected with the modified guide RNA and an mRNA or expression vector encoding the Cas protein. When co-transfected, the modified guide RNA and the mRNA or expression vector encoding the Cas protein can be provided to the cell in separate delivery systems or in a single delivery system. Alternatively, the cell may be transfected with the modified guide RNA before or after being transfected with an mRNA or expression vector encoding a Cas protein. The cell can be transfected by electroporation, microinjection, lipofection or exposure to nanoparticles or other delivery systems (as described in more detail below). In some embodiments, the mRNA or expression vector encoding the Cas protein and/or modified guide RNA are provided in nanoparticles, e.g., lipid nanoparticles.

In some embodiments, the Cas protein and the modified guide RNA are provided as a ribonucleoprotein complex (RNP). The modified guide RNA can be complexed with a Cas protein or a variant or fusion protein thereof to form a RNP for introduction into a cell. The RNP can be provided to a cell in a delivery system such as by electroporation, microinjection, virus-like particles, lipofection or exposure to nanoparticles or other delivery systems (as described in more detail below). In some embodiments, the Cas protein and/or modified guide RNA are provided in nanoparticles, e.g., lipid nanoparticles.

In some embodiments, the cell to be edited or modulated is ex vivo. In other embodiments, the cell to be edited or modulated is in vivo. The present methods can be used for editing a target region, or modulating expression of a target gene in a target region, in a nucleic acid in an ex vivo cell that was previously cultured in a medium comprising serum, where the cell was incompletely separated from the serum or one or more serum components. For example, the method may comprise transferring a cell from a cell culture medium to a reaction medium without washing the cell, or without extensive washing of the cell. In some embodiments, the modified guide RNA and the Cas protein are provided to the cell in the presence of serum or one or more serum components, such as an in vivo cell in blood, plasma or serum.

In some embodiments, the cell is a population of cells, each comprising the target region. For instance, the population of cells may be a cell culture or derived from a cell culture. The cell or population of cells may be in a cell culture medium or nuclease containing fluid before the modified guide RNA and the Cas protein are provided to the cell, and in some embodiments, the cell is washed but not completely free from the cell culture medium, or one or more components of the cell culture medium such that nucleases are still present, before the introduction of the editing components. For instance, a cell may be transferred from a cell culture medium to a reaction medium without washing the cell or without removal of substantially all the components of the cell culture medium. Alternatively, the cell or population of cells may be present in a cell culture medium at the time of providing the modified guide RNA and the Cas protein. In such embodiments, the cell culture medium can function as a reaction medium for editing or modulating a target sequence in the cell. In some embodiments, the cell is in, or is transferred from, a cell culture medium comprising serum or one or more other medium components, such one or more natural proteins of human or animal origin. In some embodiments, the cell is in, or is transferred from, a cell culture medium comprising bovine serum albumin, horse serum, or fetal bovine serum.

In some embodiments, the editing or expression modulation that results from providing the modified guide RNA to the cell is at least 10%, at least 20%, at least 25%, or at least 50% more efficient than editing or modulation caused by an unmodified guide RNA that is otherwise identical to the modified guide RNA. For instance, the present methods have a mean indel yield or mean edit yield at least 10%, at least 20%, at least 25%, or at least 50% higher than the yield obtained in a corresponding method employing an unmodified guide RNA that is otherwise identical to the modified guide RNA. In some embodiments, the editing or modulation that results from providing the modified guide RNA to the cell is at least 2-fold, at least 3-fold, or at least 5-fold more efficient than editing or modulation caused by an unmodified guide RNA that is otherwise identical to the modified guide RNA. For instance, the present methods have a mean indel yield or mean edit yield at least 2-fold, at least 3-fold, or at least 5-fold higher than a corresponding method employing an unmodified guide RNA that is otherwise identical to the modified guide RNA.

Multiplexing is contemplated in the present invention by using a plurality of modified gRNAs for a plurality of target regions. In some embodiments, two modified guide RNAs of the present application are used to edit (or modulate) two different target regions in the same cell, preferably at the same time. In some embodiments, a modified guide RNA is used to edit a first target region, and a second modified guide RNA is used to modulate the expression of a target region (which may be the same or different from the first target region), in a multiplexed manner.

In recent years, CRISPR-based technologies have emerged as a potentially revolutionary therapy (e.g., for correcting genetic defects). However, the use of CRISPR systems has been limited due to practical concerns. In particular, there is a need for methods to stabilize the guide RNA (gRNA) for in vivo delivery of CRISPR-Cas components. Prior research has investigated the use of gRNAs having chemically-modified nucleotides. As explained herein, the present disclosure is based in part on the surprising discovery that the incorporation of particular modified nucleotides at the 3′ end of a gRNA can improve the yield of Cas-mediated editing or modulation of target nucleic acids, with a pronounced improvement in cases where a gRNA and an mRNA or DNA encoding a Cas protein are introduced (e.g. co-transfection) into a cell under challenging conditions.

In some aspects, the guide RNAs disclosed herein may be particularly advantageous in applications wherein the guide RNA is introduced into a cell under one or more challenging conditions such as:
i. the cell is in a medium comprising serum (e.g., fetal bovine serum);
ii. the cell was previously cultured in a medium comprising serum, and serum is still present when the guide RNA is introduced;
iii. the cell was previously cultured in a medium comprising one or more nucleases, and the nucleases are still present when the guide RNA is introduced;
iv. the cell has a relatively high level of nuclease activity, such as relatively high expression of one or more nucleases;
v. the cell has a relatively low level of nuclease inhibitor activity, such as a relatively low expression of nuclease inhibitor;
vi. the modified guide RNA is not in a complex with a Cas protein before delivery into the cell;
vii. the cell exists in vivo; and
viii. combinations thereof where applicable.

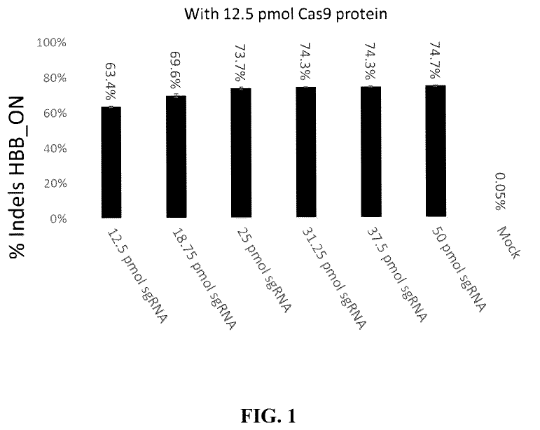

The concept of saturation is well known in the art. When a substance is at its “saturating level,” any further increase in the amount of the substance does not result in higher activities. A “sub-saturating level” is lower than the saturating level, and adding more of the substance at issue can lead to higher activities. One may determine the threshold of saturation empirically using conventional assays. For example, FIG. 1 shows the results of an assay that evaluated Cas editing activity following co-transfection with increasing amounts of a synthetic gRNA and a constant amount of Cas protein. As shown by this figure Cas-mediated editing activity plateaued at 25-31.25 pmoles of gRNA, when the level of gRNA reaches a saturation point for transfection of 0.2 million cells.

In many cases, it would be desirable to use a saturating level of the components required by a chemical reaction. However, such conditions are not always feasible, particularly in the case of therapeutics where it may not be possible or safe to treat a human or animal with a saturating level of one or more compounds. In the case of CRISPR-based therapies, transfection efficiency is typically a bottleneck that limits the effectiveness of the therapy. For example, current CRISPR-based therapies normally require co-transfection of one or more cells of a patient with a gRNA and an mRNA encoding a Cas protein. If the transfection efficiency is low, one or both components may be delivered at a level below the effective amount required for a therapeutic effect. The modified guide RNA constructs disclosed herein address this need in the art in that they typically display high levels of Cas editing activity even when transfected at a sub-saturating level. Indeed, the incorporation of one or more phosphonocarboxylate modifications at the 3′ end of a synthetic gRNA is particularly advantageous for CRISPR-based methods involving co-transfection of Cas mRNA with synthetic gRNA.

As noted above, the present disclosure also provide modified pegRNA constructs and methods which retain high levels of prime editing activity under challenging conditions, such as when transfected at sub-saturating amounts. This result is particularly surprising, as the structure of a traditional guide RNA (gRNA) is very different from that of a prime editing gRNA (pegRNA), and it was unclear, prior to the present disclosure, how chemical modifications of a pegRNA would impact its activity. In particular, pegRNAs contain additional sequences in their 3′ portions compared to typical gRNAs (i.e., a reverse transcriptase template and a primer binding site sequence) and the 3′ ends of pegRNAs perform a different function in prime editing than the 3′ ends of typical gRNAs in other CRISPR-Cas systems. Thus, phosphoribose (or other chemical) modifications at the 3′ terminus of pegRNA have the potential to interfere with the role of the primer binding site sequence, which hybridizes to the 3′ end of the nicked strand of the DNA target site, such that the reverse transcriptase recognizes the resulting RNA:DNA duplex as an acceptable substrate for primer extension of the nicked 3′ end to achieve prime editing.

Based on this understanding, one would expect that some phosphoribose modifications such as MS and MP in the RNA segment of the RNA:DNA duplex may interfere with, or reduce, the affinity of the reverse transcriptase for this duplex and thus reduce prime editing activity. Moreover, positions and/or combinations of positions where phosphoriboses are modified (such as by MS or MP) would be expected to interfere with reverse transcriptase function in prime editing and thus reduce prime editing activity. A published co-crystal structure of a complex between an RNA:DNA duplex and a portion of the duplex-complexing polypeptide fragment of the reverse transcriptase from xenotropic murine leukemia virus-related virus, a close relative of the Moloney murine leukemia virus (MMLV) whose reverse transcriptase is typically employed in prime editing, lacks the portion of the reverse transcriptase that interacts with the 3′ terminus of the RNA strand in the RNA:DNA duplex (Nowak et al., Nucl. Acids Res. 2013, 3874-3887), leaving the art with a lack of information about the RNA-protein contacts which may be important at the 3′ terminus of a pegRNA in prime editing.

The present disclosure is based in part on the surprising finding that modified gRNAs or pegRNAs which include one or more MP modifications at the 3′ end, optionally with one or more modifications at the 5′ end, can enhance Cas-mediated editing activities, particularly in cases where the modified guide RNA is transfected into a cell at a sub-saturating level. As discussed in further detail below, various designs of chemically-synthesized single guide RNAs, which can be about 100 nts long, and pegRNAs, which are typically longer, were co-transfected with a Cas protein or mRNA encoding a Cas protein in cultured human cells and enhanced activity was observed when MS or MP modifications were added to phosphoriboses at the 3′ end of the gRNA/pegRNA.

In order to evaluate the impact of various 3′ and/or 5′ end modifications, a series of synthetic gRNAs targeting the HBB gene were created by systematically incorporating MS or MP phosphoribose modifications at the 3′ ends of the gRNAs as listed in Table 1. The 5′ and 3′ end modifications are indicated in the name of each synthetic gRNA; for example, HBB-101-3×MS, 3×MP means a guide RNA for the HBB gene with three MS modifications at the 5′ end and three MP modifications at the 3′ end of the gRNA. The exact locations of the modifications are denoted by underline in the sequences shown in FIG. 1. The name also indicates the RNA length; for example, HBB-101-etc. means a strand of sgRNA targeting the HBB gene that is composed of 101 nucleotides. Likewise, HBB-99-etc. means the sgRNA strand is composed of 99 nucleotides. The difference in sequence length between these and similar lengths lies in the different number of uridines in the short polyuridine (polyU) tail at the 3′ terminus of the sgRNA, as specified by the sequences defined in Table 1. In the examples listed in Table 1, the 3′ polyU tail is composed of 3, 4, 5, 6 or 7 consecutive uridines (as a point of reference, the 3′ polyU tail on natural tracrRNAs is generally composed of 7 consecutive uridines). Also, any modification in the guide sequence is indicated after the name of the target gene and the RNA length. For example, HBB-102-11MP-3×MS, 3×MP means a guide RNA for the AUB gene composed of 102 nucleotides with three MS modifications at the 5′-end and three MP modifications at the 3′-end and comprising an MIP modification at position 11 in the guide sequence. The exact locations of the modifications are denoted by underline in the sequences shown in Table 1 (as well as Tables 2 to 4, with MP modifications in the guide sequence noted in underlined bold.

TABLE 1
Exemplary synthetic gRNAs targeting the HBB gene

| SEQ | | | Length |
|---|---|---|---|
| ID NO | sgRNA Name | Sequence (5′→3′) | (nts) |
| 1 | HBB-100_unmodified | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 2 | HBB-100_3xMS, 3xMS | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 3 | HBB-99_3xMS, 1xMP | CUUGCCCCACAGGGCAGUAAGU | 99 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUU | |
| 4 | HBB-100_3xMS, 1xMP | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 5 | HBB-100_3xMS, 2xMP | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 6 | HBB-101_3xMS, 3xMP | CUUGCCCCACAGGGCAGUAAGU | 101 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUU | |
| 7 | HBB-102_3xMS, 3xMP | CUUGCCCCACAGGGCAGUAAGU | 102 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUU | |
| 8 | HBB-102_3xMS, 4xMP | CUUGCCCCACAGGGCAGUAAGU | 102 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUU | |
| 9 | HBB-103_3xMS, 4xMP | CUUGCCCCACAGGGCAGUAAGU | 103 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUUU | |
| 10 | HBB-100_5MP_3xMS, 3xMS | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 11 | HBB-99_5MP_3xMS, 1xMP | CUUGCCCCACAGGGCAGUAAGU | 99 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUU | |
| 12 | HBB-100_5MP_3xMS, 2xMP | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 13 | HBB-101_5MP_3xMS, 3xMP | CUUGCCCCACAGGGCAGUAAGU | 101 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUU | |
| 14 | HBB-102_5MP_3xMS, 4xMP | CUUGCCCCACAGGGCAGUAAGU | 102 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUU | |
| 15 | HBB-100_11MP_3xMS, 3xMS | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 16 | HBB-100_11MP_3xMS, 1xMP | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 17 | HBB-100_11MP_3xMS, 2xMP | CUUGCCCCACAGGGCAGUAAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 18 | HBB-102_11MP_3xMS, 3xMP | CUUGCCCCACAGGGCAGUAAGU | 102 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUU | |
| 19 | HBB-103_11MP_3xMS, 4xMP | CUUGCCCCACAGGGCAGUAAGU | 103 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUUU | |
| 20 | HBB-163_unmodified | CUUGCCCCACAGGGCAGUAAGU | 163 |
| | | UUUAGAGCUAGGCCAACAUGAG | |
| | | GAUCACCCAUGUCUGCAGGGCC | |
| | | UAGCAAGUUAAAAUAAGGCUAG | |
| | | UCCGUUAUCAACUUGGCCAACA | |
| | | UGAGGAUCACCCAUGUCUGCAG | |
| | | GGCCAAGUGGCACCGAGUCGGU | |
| | | GCUUUUUUU | |
| 21 | HBB-163_3xMS, 1xMP | CUUGCCCCACAGGGCAGUAAGU | 163 |
| | | UUUAGAGCUAGGCCAACAUGAG | |
| | | GAUCACCCAUGUCUGCAGGGCC | |
| | | UAGCAAGUUAAAAUAAGGCUAG | |
| | | UCCGUUAUCAACUUGGCCAACA | |
| | | UGAGGAUCACCCAUGUCUGCAG | |
| | | GGCCAAGUGGCACCGAGUCGGU | |
| | | GCUUUUUUU | |
| 22 | HBB-163_3xMS, 2xMP | CUUGCCCCACAGGGCAGUAAGU | 163 |
| | | UUUAGAGCUAGGCCAACAUGAG | |
| | | GAUCACCCAUGUCUGCAGGGCC | |
| | | UAGCAAGUUAAAAUAAGGCUAG | |
| | | UCCGUUAUCAACUUGGCCAACA | |
| | | UGAGGAUCACCCAUGUCUGCAG | |
| | | GGCCAAGUGGCACCGAGUCGGU | |
| | | GCUUUUUUU | |
| 23 | HBB-163_3xMS, 3xMP | CUUGCCCCACAGGGCAGUAAGU | 163 |
| | | UUUAGAGCUAGGCCAACAUGAG | |
| | | GAUCACCCAUGUCUGCAGGGCC | |
| | | UAGCAAGUUAAAAUAAGGCUAG | |
| | | UCCGUUAUCAACUUGGCCAACA | |
| | | UGAGGAUCACCCAUGUCUGCAG | |
| | | GGCCAAGUGGCACCGAGUCGGU | |
| | | GCUUUUUUU | |
| 24 | HBB-163_3xMS, 4xMP | CUUGCCCCACAGGGCAGUAAGU | 163 |
| | | UUUAGAGCUAGGCCAACAUGAG | |
| | | GAUCACCCAUGUCUGCAGGGCC | |
| | | UAGCAAGUUAAAAUAAGGCUAG | |
| | | UCCGUUAUCAACUUGGCCAACA | |
| | | UGAGGAUCACCCAUGUCUGCAG | |
| | | GGCCAAGUGGCACCGAGUCGGU | |
| | | GCUUUUUUU | |

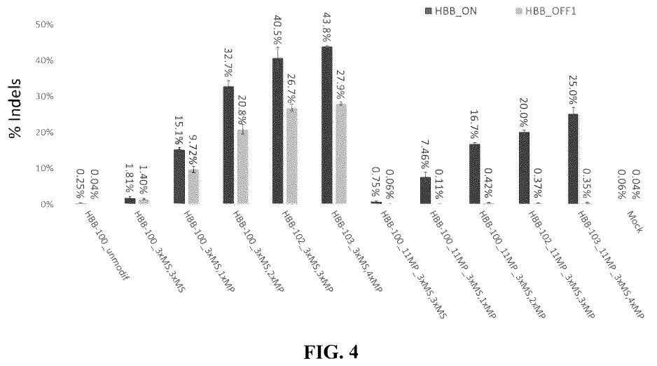

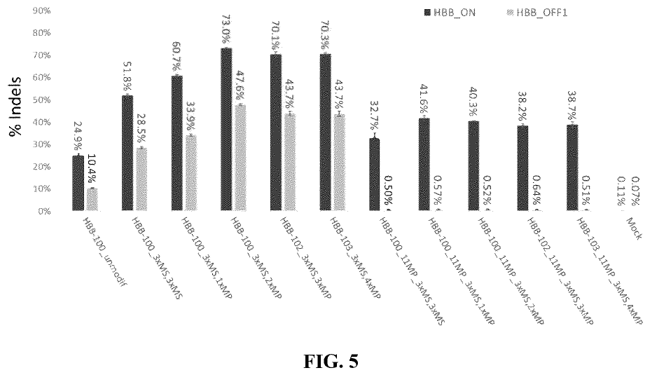

The number and types of chemical modifications at the 3′ end of gRNAs can substantially improve their efficacy for DNA editing under conditions wherein a sub-saturating amount of the gRNA is delivered into a cell (e.g., by nucleofection). This benefit is especially pronounced in methods using gRNA co-transfected with an mRNA encoding a Cas protein, as opposed to being co-transfected in a complex with Cas protein as a ribonucleoprotein (RNP) complex. The number and types of chemical modifications incorporated into a gRNA can also improve the editing efficiency of a Cas RNP complex, illustrated by the data provided herein regarding transfection of cells suspended in growth media comprising serum (which is known to contain nucleases). See, e.g., FIGS. 4 and 5. The experimental data described herein also shows that certain chemical modifications and certain sequence positions in the transfected gRNA sequence can be especially advantageous for enhancing editing yields, such as by incorporating one or more MP modifications at consecutive 3′ terminal phosphoriboses on the 3′ end of a gRNA.

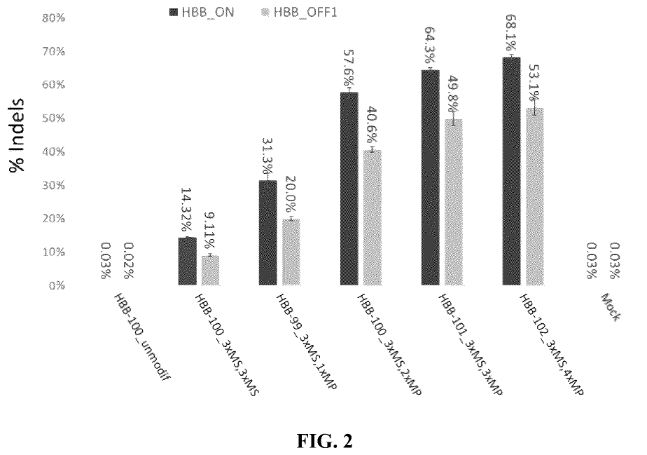

Any of the 5′ and 3′ end modifications described herein may optionally be combined with modifications in the guide sequence of a gRNA that enhance target specificity (as described, e.g., in U.S. Pat. No. 10,767,175). For example, MP modifications on the 3′ end (such as MP at the second nucleotide from the 3′ end, which means the first internucleotide linkage from the 3′ end comprises a phosphonoacetate) may be combined with MP or other modifications at position 5 or 11 (counting from the 5′ end of the guide sequence in a 20-nucleotide guide sequence) in the guide sequence portion of a gRNA or pegRNA, as illustrated in Table 1 and as tested in FIGS. 2-5.

The chemical modifications may be incorporated during chemical synthesis of gRNAs by using chemically-modified phosphoramidites at select cycles of amidite coupling to produce the desired sequence. Once synthesized, the chemically-modified gRNA is used in the same manner as unmodified gRNA for gene editing or regulation. A preferred embodiment is to co-transfect the chemically-modified synthetic gRNA with an mRNA or DNA encoding a Cas protein. Chemical modifications enhance the activity of the gRNA in transfected cells, including when delivered by electroporation, lipofection or exposure of live cells or tissues to nanoparticles charged with gRNA and/or an mRNA encoding a Cas protein.

Exemplary synthetic pegRNAs are shown below in Tables 2 and 3. These pegRNAs were modified by systematically incorporating MS or MP phosphoribose modifications at the 3′ ends. The 5′ and 3′ end modifications are indicated in the name of each synthetic pegRNA, which also indicates the target gene. For example, “EMX1-peg-3×MS, 3×MP” refers to a pegRNA for the EMX1 gene with three MS modifications at the 5′ end and three MP modifications at the 3′ end of the pegRNA. The exact locations of the modifications are denoted by underlining in the sequences shown in Table 2. Some of the pegRNA designs have a short polyuridine tract (i.e., a polyU tail) added to the 3′ terminus, as indicated by “+3′UU”, “+3′UUU”, or “+3′UUUU” in the pegRNA name.

TABLE 2
Exemplary synthetic pegRNAs targeting the EMX1 gene

| SEQ | | | Length |
|---|---|---|---|
| ID NO | sgRNA Name | Sequence (5′→3′) | (nts) |
| 25 | EMX1-peg_3xMS, unmod | GAGUCCGAGCAGAAGAAGAAGU | 124 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGAC | |
| 26 | EMX1-peg_3xMS, 3xMS | GAGUCCGAGCAGAAGAAGAAGU | 124 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGAC | |
| 27 | EMX1-peg_3xMS, 1xMP | GAGUCCGAGCAGAAGAAGAAGU | 124 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGAC | |
| 28 | EMX1-peg_3xMS, 2xMP | GAGUCCGAGCAGAAGAAGAAGU | 124 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGAC | |
| 29 | EMX1-peg_3xMS, 3xMP | GAGUCCGAGCAGAAGAAGAAGU | 124 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGAC | |
| 30 | EMX1-ng_3xMS, 3xMS | GCCGUUUGUACUUUGUCCUCGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 41 | EMX1-peg + 3′UU- | GAGUCCGAGCAGAAGAAGAAGU | 126 |
| | 3xMS, 3xMS | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUU | |
| 42 | EMX1-peg + 3′UUUU- | GAGUCCGAGCAGAAGAAGAAGU | 128 |
| | 3xMS, 3xMS | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUUUU | |
| 43 | EMX1-peg + 3′UU- | GAGUCCGAGCAGAAGAAGAAGU | 126 |
| | 3xMS, 1xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUU | |
| 44 | EMX1-peg + 3′UU- | GAGUCCGAGCAGAAGAAGAAGU | 126 |
| | 3xMS, 2xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUU | |
| 45 | EMX1-peg + 3′UUU- | GAGUCCGAGCAGAAGAAGAAGU | 127 |
| | 3xMS, 2xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUUU | |
| 46 | EMX1-peg + 3′UUUU- | GAGUCCGAGCAGAAGAAGAAGU | 128 |
| | 3xMS, 3xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUUUU | |
| 47 | EMX1-peg + 3′UUUU- | GAGUCCGAGCAGAAGAAGAAGU | 128 |
| | 3xMS, 2xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUUUU | |
| 48 | EMX1-peg + 3′UUUU- | GAGUCCGAGCAGAAGAAGAAGU | 128 |
| | 3xMS, 1xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCAUGGGAGCACUUCU | |
| | | UCUUCUGCUCGGACUUUU | |

TABLE 3
Exemplary synthetic pegRNAs targeting the EMXI gene.

| SEQ | | | Length |
|---|---|---|---|
| ID NO | sgRNA Name | Sequence (5′→3′) | (nts) |
| 31 | RUNX1-peg_3xMS, | GCAUUUUCAGGAGGAAGCGAGU | 129 |
| | unmod | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAU | |
| 32 | RUNX1-peg_3xMS, 3xMS | GCAUUUUCAGGAGGAAGCGAGU | 129 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAU | |
| 33 | RUNX1-peg_3xMS, 1xMP | GCAUUUUCAGGAGGAAGCGAGU | 129 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAU | |
| 34 | RUNX1-peg_3xMS, 2xMP | GCAUUUUCAGGAGGAAGCGAGU | 129 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAU | |
| 35 | RUNX1-peg_3xMS, 3xMP | GCAUUUUCAGGAGGAAGCGAGU | 129 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAU | |
| 36 | RUNX1-ng_3xMS, 3xMS | AUGAAGCACUGUGGGUACGAGU | 100 |
| | | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 49 | RUNX1-peg + UU-3xMS, | GCAUUUUCAGGAGGAAGCGAGU | 131 |
| | 3xMS | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAUUU | |
| 50 | RUNX1-peg + UUUU- | GCAUUUUCAGGAGGAAGCGAGU | 133 |
| | 3xMS, 3xMS | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAUUUU | |
| | | U | |
| 51 | RUNX1-peg + UU-3xMS, | GCAUUUUCAGGAGGAAGCGAGU | 131 |
| | 2xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAUUU | |
| 52 | RUNX1-peg + UUUU- | GCAUUUUCAGGAGGAAGCGAGU | 133 |
| | 3xMS, 3xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAUUUU | |
| | | U | |
| 53 | RUNX1-peg + UUUU- | GCAUUUUCAGGAGGAAGCGAGU | 133 |
| | 3xMS, 2xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAUUUU | |
| | | U | |
| 54 | RUNX1-peg + UUUU- | GCAUUUUCAGGAGGAAGCGAGU | 133 |
| | 3xMS, 1xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUGUCUGAAGCCAUC | |
| | | CAUGCUUCCUCCUGAAAAUUUU | |
| | | U | |

TABLE 4
Exemplary synthetic gRNAs targeting the IL2RG gene

| SEQ | | | |
|---|---|---|---|
| ID | | | Length |
| NO | sgRNA Name | Sequence (5′→3′) | (nts) |
| 37 | IL2RG-100_3xMS, | UGGUAAUGAUGGCUUCAACAGU | 100 |
| | 3xMS | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 38 | IL2RG-100_3xMS, | UGGUAAUGAUGGCUUCAACAGU | 100 |
| | 2xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUU | |
| 39 | IL2RG-102 3xMS, | UGGUAAUGAUGGCUUCAACAGU | 102 |
| | 3xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUU | |
| 40 | IL2RG-103 3xMS, | UGGUAAUGAUGGCUUCAACAGU | 103 |
| | 4xMP | UUUAGAGCUAGAAAUAGCAAGU | |
| | | UAAAAUAAGGCUAGUCCGUUAU | |
| | | CAACUUGAAAAAGUGGCACCGA | |
| | | GUCGGUGCUUUUUUU | |

As demonstrated by the examples described below, the use of chemical modifications at the 3′ end of pegRNAs substantially improves the efficacy of synthetic pegRNAs with prime editors (with respect to pegRNAs that are unmodified at the 3′ end). The use of synthetic pegRNAs for prime editing can be preferred when aiming to limit the duration of editing activity, as opposed to a sustained editing activity when pegRNAs and prime editors are constitutively expressed in cells transfected with DNA vectors as originally reported in the literature (see Anzalone et al. 2019). The present disclosure further demonstrates that data certain chemical modifications and certain sequence positions in a pegRNA sequence can be especially advantageous, in some aspects, such as incorporating two MP modifications at consecutive 3′ terminal phosphoriboses on a pegRNA strand that terminates with a primer binding segment at the 3′ terminus (without adding a downstream polyU tail to the 3′ terminus).

### A. Exemplary CRISPR/Cas Systems

The CRISPR/Cas system of genome modification includes a Cas protein (e.g., Cas9 nuclease), and a DNA-targeting RNA (e.g., modified gRNA) containing a guide sequence that targets the Cas protein to the target DNA, and a scaffold region that interacts with the Cas protein (e.g., tracrRNA). In some instances, a variant of a Cas protein such as a Cas9 mutant containing one or more of the following mutations: D10A, H840A, D839A, and H863A, can be used. In other instances, a fragment of a Cas protein or a variant thereof with desired properties (e.g., capable of generating single- or double-strand breaks and/or modulating gene expression) can be used. A donor repair template may be used in some CRISPR applications, which, for example, can include a nucleotide sequence encoding a reporter polypeptide such as a fluorescent protein or an antibiotic resistance marker, and homology arms that are homologous to the target DNA and flank the site of gene modification. Alternatively, the donor repair template can be a single-stranded oligodeoxynucleotide (ssODN). In some aspects, a CRISPR/CAS system may include a Cas protein capable of acting as a prime editor (e.g., a fusion protein comprising a Cas protein which displays nickase activity fused to a reverse transcriptase protein or domain thereof). A prime editor may be used with a pegRNA, which incorporates a reverse transcriptase template containing one or more edits to the sequence of a target nucleic acid, in order to modify the sequence of the target nucleic acid by a process referred to as prime editing.

1. Cas proteins and Variants Thereof

The CRISPR (Clustered Regularly Interspaced Short Palindromic Repeats)/Cas (CRISPR-associated protein) nuclease system was discovered in bacteria but has been used in eukaryotic cells (e.g. mammalian) for genome editing/modulation of gene expression. It is based on part of the adaptive immune response of many bacteria and archaea. When a virus or plasmid invades such a microbe, segments of the invader's DNA are incorporated into a CRISPR locus (or “CRISPR array”) in the microbial genome. Expression of the CRISPR locus produces non-coding CRISPR RNAs (crRNA). In Type II CRISPR systems, the crRNA then associates, through a region of partial complementarity, with another type of RNA called tracrRNA to guide the Cas (e.g., Cas9) protein to a region homologous to the crRNA in the target DNA called a “protospacer.” The Cas (e.g., Cas9) protein cleaves the DNA to generate blunt ends at the double-strand break at sites specified by a 20-nucleotide guide sequence contained within the crRNA transcript. The Cas (e.g., Cas9) protein requires both the crRNA and the tracrRNA for site-specific DNA recognition and cleavage. This system has been engineered such that the crRNA and tracrRNA can be combined into one molecule (a single guide RNA or “sgRNA”) (see, e.g., Jinek et al. (2012) Science, 337:816-821; Jinek et al. (2013) eLife, 2:e00471; Segal (2013) eLife, 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments, the Cas protein has DNA cleavage activity. The Cas protein can direct cleavage of one or both strands at a location in a target DNA sequence. For example, the Cas protein can be a nickase having one or more inactivated catalytic domains that cleaves a single strand of a target DNA sequence (e.g., as in the case of a prime editor Cas protein).

Non-limiting examples of Cas proteins include Cas1, Cas1B, Cas2, Cas3, Cas4, Cas5, Cas6, Cas7, Cas8, Cas9 (also known as Csn1 and Csx12), Cas10, Cas11, Cas12, Cas13, Cas14, CasΦ, CasX, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, homologs thereof, variants thereof, fragments thereof, mutants thereof, and derivatives thereof. There are at least six types of Cas protein (Types I through VI), and at least 33 subtypes (see, e.g., Makarova et al., Nat. Rev. Microbiol., 2020, 18:2, 67-83). Type II Cas proteins include Cas1, Cas2, Csn2, and Cas9. Cas proteins are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. CRISPR-related endonucleases that are useful in aspects of the present disclosure are disclosed, e.g., in U.S. Pat. Nos. 9,267,135; 9,745,610; and 10,266,850.

Cas proteins, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacterial species.

“Cas12” (comprising variants Cas12a (also known as Cpf1), Cas12b, c2c1, c2c3, CasX, and CasY) refers to an RNA-guided double-stranded DNA-binding nuclease protein containing a mixed alpha/beta domain, a RuvC-I followed by a helical region, a RuvC-II and a zinc finger-like domain or nickase protein. Wild-type Cas12 nucleases produce staggered, 5′ overhangs on the dsDNA target sequence and do not require a tracrRNA. Cas12 and its variants recognize a 5′ AT-rich PAM sequence on the target dsDNA. An insert domain, called Nuc, of the Cas12a protein has been demonstrated to be responsible for target strand cleavage. The Cas12 enzyme can comprise one or more catalytic domains of a Cas12 protein derived from bacteria belonging to the group consisting of Francisella and Prevotella.

Useful variants of the Cas9 protein can include a single inactive catalytic domain, such as a RuvC⁻ or HNH⁻ enzymes, both of which are nickases. Such Cas proteins are useful, e.g., in the context of prime editing. A Cas9 nickase has only one active functional domain and can cut only one strand of the target DNA, thereby creating a single-strand break or nick. In some embodiments, the Cas protein is a mutant Cas9 nuclease having at least a D10A mutation, and is a Cas9 nickase. In other embodiments, the Cas protein is a mutant Cas9 nuclease having at least a H840A mutation, and is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A staggered double-nick-induced double-strand break can be repaired by NHEJ or HDR (Ran et al., 2013, Cell, 154:1380-1389; Anzalone et al. Nature 576:7785, 2019, 149-15). This gene editing strategy favors HDR and decreases the frequency of indel mutations as byproducts. Non-limiting examples of Cas9 nucleases or nickases are described in, for example, U.S. Pat. Nos. 8,895,308; 8,889,418; 8,865,406; 9,267,135; and 9,738,908; and in U.S. Patent Application Pub. No. 2014/0186919. The Cas9 nuclease or nickase can be codon-optimized for the target cell or target organism.

In some embodiments, the Cas protein can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Pub. No. WO 2013/176772. The dCas9 enzyme can contain a mutation at D10, E762, H983 or D986, as well as a mutation at H840 or N863. In some instances, the dCas9 enzyme contains a D10A or D1ON mutation. Also, the dCas9 enzyme can include a H840A, H840Y, or H840N. In some embodiments, the dCas9 enzyme used in aspects of the present disclosure comprises D10A and H840A; D10A and H840Y; D10A and H840N; D1ON and H840A; D10N and H840Y; or D10N and H840N substitutions. The substitutions can be conservative or non-conservative substitutions to render the Cas9 polypeptide catalytically inactive and able to bind to target DNA.

The dCas9 polypeptide is catalytically inactive and lacks nuclease activity. In some instances, the dCas9 enzyme or a variant or fragment thereof can block transcription of a target sequence, and in some cases, block RNA polymerase. In other instances, the dCas9 enzyme or a variant or fragment thereof can activate transcription of a target sequence, for example, when fused to a transcriptional activator polypeptide. In some embodiments, the Cas protein or protein variants comprise one or more NLS sequences.

In some embodiments, the Cas protein can be a fusion protein which comprises one or more Cas nuclease domains fused to one or more heterologous functional domains of a second protein, with an optional intervening linker, wherein the linker does not interfere with activity of the fusion protein. Heterologous in this context means the functional domain is from a protein other than a Cas protein. In some embodiments, the heterologous functional domain comprise an enzymatic domain and/or a binding domain. In some embodiments, the heterologous enzymatic domain is a nuclease, a nickase, a recombinase, a deaminase, a methyltransferase, a polymerase, a reverse transcriptase, a methylase, an acetylase, an acetyltransferase, a transcriptional activator, or a transcriptional repressor domain. In some embodiments, the heterologous enzymatic domain comprises base editing activity, nucleotide deaminase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

In some embodiments, the Cas protein comprises a heterologous functional domain which is a base editor, such as a cytidine deaminase domain, for example, from the apolipoprotein B mRNA-editing enzyme, catalytic polypeptide-like (APOBEC) family of deaminases, including APOBEC1, APOBEC2, APOBEC3A, APOBEC3B, APOBEC3C, APOBEC3D/E, APOBEC3F, APOBEC3G, APOBEC3H, or APOBEC4; activation-induced cytidine deaminase (AID), e.g., activation induced cytidine deaminase (AICDA); cytosine deaminase 1 (CDA1) or CDA2; or cytosine deaminase acting on tRNA (CDAT). In some embodiments, the heterologous functional domain is a deaminase that modifies adenosine DNA bases, e.g., the deaminase is an adenosine deaminase 1 (ADA1), ADA2; adenosine deaminase acting on RNA 1 (ADAR1), ADAR2, ADAR3; adenosine deaminase acting on tRNA 1 (ADAT1), ADAT2, ADAT3; and naturally occurring or engineered tRNA-specific adenosine deaminase (TadA). In some embodiments, the heterologous functional domain is a biological tether. In some embodiments, the biological tether is MS2, Csy4 or lambda N protein. In some embodiments, the heterologous functional domain is FokI.

In some embodiments, the Cas protein comprises a heterologous functional domain which is an enzyme, domain, or peptide that inhibits or enhances endogenous DNA repair or base excision repair (BER) pathways, for example, uracil DNA glycosylase inhibitor (UGI) that inhibits uracil DNA glycosylase (UDG, also known as uracil N-glycosylase, or UNG) mediated excision of uracil to initiate BER; or DNA end-binding proteins such as Gam from the bacteriophage Mu.

In some embodiments, the Cas protein comprises a heterologous functional domain which is a transcriptional activation domain, for example, a VP64 domain, a p65 domain, a MyoD1 domain, or a HSF1 domain. In some embodiments, the Cas protein comprises a heterologous functional domain which is a transcriptional repression domain, for example, a Krueppel-associated box (KRAB) domain, an ERF repressor domain (ERD), a mSin3A interaction domain (SID) domain, a SID4X domain, a NuE domain, or a NcoR domain. In some embodiments, the Cas protein comprises a heterologous functional domain which is a nuclease domain, for example, a Fok1 domain. In some embodiments, the Cas protein comprises a transcriptional silencer domain, for example, Heterochromatin Protein 1 (HP1), e.g., HPla or HP1D. In some embodiments, the heterologous functional domain of the Cas protein is an enzyme that modifies the methylation state of DNA. In some embodiments, the enzyme that modifies the methylation state of DNA is a DNA methyltransferase (DNMT) or a TET protein. In some embodiments, the TET protein is TET1. In some embodiments, the heterologous functional domain of the Cas protein is an enzyme that modifies a histone subunit. In some embodiments, the enzyme that modifies a histone subunit is a histone acetyltransferase (HAT), histone deacetylase (HDAC), histone methyltransferase (HMT), or histone demethylase.

For gene regulation (e.g., modulating transcription of target DNA), a nuclease-deficient Cas protein, such as but not limited to dCas9, can be used for transcriptional activation or transcriptional repression. Methods of inactivating gene expression using a nuclease-null Cas protein are described, for example, in Larson et al., Nat. Protoc., 2013, 8(11):2180-2196.

In some embodiments, the Cas protein comprises one or more nuclear localization signal (NLS) domains. The one or more NLS domain(s) may be positioned at or near or in proximity to a terminus of the effector protein (e.g., C2c2) and if two or more NLSs, each of the two may be positioned at or near or in proximity to a terminus of the effector protein (e.g., C2c2).

In some embodiments, a nucleotide sequence encoding the Cas protein is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell.

Depending on the target cell/expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

The Cas protein can be introduced into a cell (e.g., a cell such as a primary cell for ex vivo therapy, or an in vivo cell such as in a patient) as a Cas polypeptide, an mRNA encoding a Cas polypeptide, or a recombinant expression vector comprising a nucleotide sequence encoding a Cas polypeptide.

2. Chemically-Modified Guide RNA (gRNA)

The modified gRNAs for use in the CRISPR/Cas system of genome modification typically include a guide sequence that is complementary to a target nucleic acid sequence and a scaffold region that interacts with a Cas protein.

The guide sequence of the modified guide RNA can be any polynucleotide sequence having sufficient complementarity with a target polynucleotide sequence (e.g., target DNA sequence) to hybridize with the target sequence and direct sequence-specific binding of a CRISPR complex to the target sequence. In some embodiments, the degree of complementarity between a guide sequence of the modified guide RNA and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif), SOAP (available at soap.genomics.org.cn), and Maq (available at maq.sourceforge.net). In some embodiments, a guide sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some instances, a guide sequence is about 20 nucleotides in length. In other instances, a guide sequence is about 15 nucleotides in length. In other instances, a guide sequence is about 25 nucleotides in length. The ability of a guide sequence to direct sequence-specific binding of a CRISPR complex to a target sequence may be assessed by any suitable assay. Binding can be assessed directly, or indirectly by using, e.g., editing or cleavage as a proxy. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of editing or cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

The nucleotide sequence of a guide RNA can be selected using any of the web-based software described above. Considerations for selecting a DNA-targeting RNA include the PAM sequence for the Cas protein (e.g., Cas9 polypeptide) to be used, and strategies for minimizing off-target modifications. Tools, such as the CRISPR Design Tool, can provide sequences for preparing the modified gRNA, for assessing target modification efficiency, and/or assessing cleavage at off-target sites. Another consideration for selecting the sequence of a modified guide RNA includes reducing the degree of secondary structure within the guide sequence. Secondary structure may be determined by any suitable polynucleotide folding algorithm. Some programs are based on calculating the minimal Gibbs free energy. Examples of suitable algorithms include mFold (Zuker and Stiegler, Nucleic Acids Res, 9 (1981), 133-148), UNAFold package (Markham et al., Methods Mol Biol, 2008, 453:3-31) and RNAfold form the ViennaRNA Package.

One or more nucleotides of the guide sequence and/or one or more nucleotides of the scaffold region of the modified guide RNA can be a modified nucleotide. For instance, a guide sequence that is about 20 nucleotides in length may have 1 or more, e.g., 1, 2, 3, 4, 5, 6, 7, 8, 9, 10, 11, 12, 13, 14, 15, 16, 17, 18, 19, 20, or more modified nucleotides. In some cases, the guide sequence includes at least 2, 3, 4, 5, 6, 7, 8, 9, 10, or more modified nucleotides. In other cases, the guide sequence includes at least 2, 3, 4, 5, 6, 7, 8, 9, 10, 11, 12, 13, 14, 15, 16, 17, 19, 20, or more modified nucleotides. The modified nucleotides can be located at any nucleic acid position of the guide sequence. In other words, the modified nucleotides can be at or near the first and/or last nucleotide of the guide sequence, and/or at any position in between. For example, for a guide sequence that is 20 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, and/or position 20 of the guide sequence. In certain instances, from about 10% to about 30%, e.g., about 10% to about 25%, about 10% to about 20%, about 10% to about 15%, about 15% to about 30%, about 20% to about 30%, or about 25% to about 30% of the guide sequence can comprise modified nucleotides. In other instances, from about 10% to about 30%, e.g., about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% of the guide sequence can comprise modified nucleotides.

In some embodiments, the scaffold region of the modified guide RNA contains one or more modified nucleotides. For example, a scaffold region that is about 80 nucleotides in length may have 1 or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 76, 77, 78, 79, 80, or more modified nucleotides. In some instances, the scaffold region includes at least 2, 3, 4, 5, 6, 7, 8, 9, 10, or more modified nucleotides. In other instances, the scaffold region includes at least 2, 3, 4, 5, 6, 7, 8, 9, 10, 11, 12, 13, 14, 15, 16, 17, 19, 20, or more modified nucleotides. The modified nucleotides can be located at any nucleic acid position of the scaffold region. For example, the modified nucleotides can be at or near the first and/or last nucleotide of the scaffold region, and/or at any position in between. For example, for a scaffold region that is about 80 nucleotides in length, the one or more modified nucleotides can be located at nucleic acid position 1, position 2, position 3, position 4, position 5, position 6, position 7, position 8, position 9, position 10, position 11, position 12, position 13, position 14, position 15, position 16, position 17, position 18, position 19, position 20, position 21, position 22, position 23, position 24, position 25, position 26, position 27, position 28, position 29, position 30, position 31, position 32, position 33, position 34, position 35, position 36, position 37, position 38, position 39, position 40, position 41, position 42, position 43, position 44, position 45, position 46, position 47, position 48, position 49, position 50, position 51, position 52, position 53, position 54, position 55, position 56, position 57, position 58, position 59, position 60, position 61, position 62, position 63, position 64, position 65, position 66, position 67, position 68, position 69, position 70, position 71, position 72, position 73, position 74, position 75, position 76, position 77, position 78, position 79, and/or position 80 of the sequence. In some instances, from about 1% to about 10%, e.g., about 1% to about 8%, about 1% to about 5%, about 5% to about 10%, or about 3% to about 7% of the scaffold region can comprise modified nucleotides. In other instances, from about 1% to about 10%, e.g., about 1%, about 2%, about 3%, about 4%, about 5%, about 6%, about 7%, about 8%, about 9%, or about 10% of the scaffold region can comprise modified nucleotides.

The modified nucleotides of the guide RNA can include a modification in the ribose (e.g., sugar) group, phosphate group, nucleobase, or any combination thereof. In some embodiments, the modification in the ribose group comprises a modification at the 2′ position of the ribose.

In some embodiments, the modified nucleotide includes a 2′ fluoro-arabino nucleic acid, tricycle-DNA (tc-DNA), peptide nucleic acid, cyclohexene nucleic acid (CeNA), locked nucleic acid (LNA), ethylene-bridged nucleic acid (ENA), xeno nucleic acid (XNA), a phosphodiamidate morpholino, or a combination thereof.

Modified nucleotides or nucleotide analogues can include sugar- and/or backbone-modified ribonucleotides (i.e., include modifications to the phosphate-sugar backbone). For example, the phosphodiester linkages of a native or natural RNA may be modified to include at least one of a nitrogen or sulfur heteroatom. In some backbone-modified ribonucleotides the phosphoester group connecting to adjacent ribonucleotides may be replaced by a modified group, e.g., of phosphorothioate group. In preferred sugar-modified ribonucleotides, the 2′ moiety is a group selected from H, OR, R, halo, SH, SR, NH₂, NHR, NR₂ or ON, wherein R is C₁-C₆ alkyl, alkenyl or alkynyl and halo is F, Cl, Br or I.

In some embodiments, the modified nucleotide contains a sugar modification. Non-limiting examples of sugar modifications include 2′-deoxy-2′-fluoro-oligoribonucleotide (2′-fluoro-2′-deoxycytidine-5′-triphosphate, 2′-fluoro-2′-deoxyuridine-5′-triphosphate), 2′-deoxy-2′-deamine oligoribonucleotide (2′-amino-2′-deoxycytidine-5′-triphosphate, 2′-amino-2′-deoxyuridine-5′-triphosphate), 2′-O-alkyl oligoribonucleotide, 2′-deoxy-2′-C-alkyl oligoribonucleotide (2′-O-methylcytidine-5′-triphosphate, 2′-methyluridine-5′-triphosphate), 2′-C-alkyl oligoribonucleotide, and isomers thereof (2′-aracyti dine-5′-triphosphate, 2′-arauridine-5′-triphosphate), azidotriphosphate (2′-azido-2′-deoxycytidine-5′-triphosphate, 2′-azido-2′-deoxyuridine-5′-triphosphate), and combinations thereof.

In some embodiments, the modified guide RNA contains one or more 2′-fluoro, 2′-amino and/or 2′-thio modifications. In some instances, the modification is a 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, 5-amino-allyl-uridine, 5-bromo-uridine, 5-iodo-uridine, 5-methyl-cytidine, ribo-thymidine, 2-aminopurine, 2′-amino-butyryl-pyrene-uridine, 5-fluoro-cytidine, and/or 5-fluoro-uridine.

There are more than 96 naturally occurring nucleoside modifications found on mammalian RNA. See, e.g., Limbach et al., Nucleic Acids Research, 22(12):2183-2196 (1994). The preparation of nucleotides and modified nucleotides and nucleosides are well-known in the art and described in, e.g., U.S. Pat. Nos. 4,373,071, 4,458,066, 4,500,707, 4,668,777, 4,973,679, 5,047,524, 5,132,418, 5,153,319, 5,262,530, and 5,700,642. Numerous modified nucleosides and modified nucleotides that are suitable for use as described herein are commercially available. The nucleoside can be an analogue of a naturally occurring nucleoside. In some cases, the analogue is dihydrouridine, methyladenosine, methylcytidine, methyluridine, methylpseudouridine, thiouridine, deoxycytodine, and deoxyuridine.

In some cases, the modified guide RNA described herein includes a nucleobase-modified ribonucleotide, i.e., a ribonucleotide containing at least one non-naturally occurring nucleobase instead of a naturally occurring nucleobase. Non-limiting examples of modified nucleobases which can be incorporated into modified nucleosides and modified nucleotides include m5C (5-methylcytidine), m5U (5-methyluridine), m6A (N6-methyladenosine), s2U (2-thiouridine), Um (2′-O-methyluridine), m1A (1-methyl adenosine), m2A (2-methyladenosine), Am (2-1-O-methyladenosine), ms2m6A (2-methylthio-N6-methyladenosine), i6A (N6-isopentenyl adenosine), ms2i6A (2-methylthio-N6-isopentenyladenosine), io6A (N6-(cis-hydroxyisopentenyl) adenosine), ms2io6A (2-methylthio-N6-(cis-hydroxyisopentenyl)adenosine), g6A (N6-glycinylcarbamoyladenosine), t6A (N6-threonyl carbamoyladenosine), ms2t6A (2-methylthio-N6-threonyl carbamoyladenosine), m6t6A (N6-methyl-N6-threonylcarbamoyladenosine), hn6A(N6-hydroxynorvalylcarbamoyl adenosine), ms2hn6A (2-methylthio-N6-hydroxynorvalyl carbamoyladenosine), Ar(p) (2-O-ribosyladenosine(phosphate)), I (inosine), ml1 (1-methylinosine), m′Im (1,2′-O-dimethylinosine), m3C (3-methylcytidine), Cm (2T-O-methylcytidine), s2C (2-thiocytidine), ac4C (N4-acetylcytidine), f5C (5-fonnylcytidine), m5Cm (5,2-O-dimethylcytidine), ac4Cm (N4acetyl2TOmethylcytidine), k2C (lysidine), m1G (1-methylguanosine), m2G (N2-methylguanosine), m7G (7-methylguanosine), Gm (2′-O-methylguanosine), m22G (N2,N2-dimethylguanosine), m2Gm (N2,2′-O-dimethylguanosine), m22Gm (N2,N2,2′-O-trimethylguanosine), Gr(p) (2′-O-ribosylguanosine(phosphate)), yW (wybutosine), o2yW (peroxywybutosine), OHyW (hydroxywybutosine), OHyW* (undermodified hydroxywybutosine), imG (wyosine), mimG (methylguanosine), Q (queuosine), oQ (epoxyqueuosine), galQ (galactosyl-queuosine), manQ (mannosyl-queuosine), preQo (7-cyano-7-deazaguanosine), preQi (7-aminomethyl-7-deazaguanosine), G (archaeosine), D (dihydrouridine), m5Um (5,2′-O-dimethyluridine), s4U (4-thiouridine), m5s2U (5-methyl-2-thiouridine), s2Um (2-thio-2′-O-methyluridine), acp3U (3-(3-amino-3-carboxypropyl)uridine), ho5U (5-hydroxyuridine), mo5U (5-methoxyuridine), cmo5U (uridine 5-oxyacetic acid), mcmo5U (uridine 5-oxyacetic acid methyl ester), chm5U (5-(carboxyhydroxymethyl)uridine)), mchm5U (5-(carboxyhydroxymethyl)uridine methyl ester), mcm5U (5-methoxycarbonyl methyluridine), mcm5Um (S-methoxycarbonylmethyl-2-O-methyluridine), mcm5s2U (5-methoxycarbonylmethyl-2-thiouridine), nm5 s2U (5-aminomethyl-2-thiouridine), mnm5U (5-methylaminomethyluridine), mnm5s2U (5-methylaminomethyl-2-thiouridine), mnm5se2U (5-methylaminomethyl-2-selenouridine), ncm5U (5-carbamoylmethyl uridine), ncm5Um (5-carbamoylmethyl-2′-O-methyluridine), cmnm5U (5-carboxymethylaminomethyluridine), cnmm5Um (5-carboxymethylaminomethyl-2-L-O-methyluridine), cmnm5s2U (5-carboxymethylaminomethyl-2-thiouridine), m62A (N6,N6-dimethyladenosine), Tm (2′-O-methylinosine), m4C (N4-methylcytidine), m4Cm (N4,2-O-dimethylcytidine), hm5C (5-hydroxymethylcytidine), m3U (3-methyluridine), cm5U (5-carboxymethyluridine), m6Am (N6,T-0-dimethyladenosine), m62Am (N6,N6,O-2-trimethyladenosine), m2′7G (N2,7-dimethylguanosine), m2′2′7G (N2,N2,7-trimethylguanosine), m3Um (3,2T-O-dimethyluridine), m5D (5-methyldihydrouridine), f5Cm (5-formyl-2′-O-methylcytidine), m1Gm (1,2′-O-dimethylguanosine), m′Am (1,2-O-dimethyl adenosine)irinomethyluridine), tm5s2U (S-taurinomethyl-2-thiouridine)), imG-14 (4-demethyl guanosine), imG2 (isoguanosine), or ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxy cytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and combinations thereof.

In some embodiments, the phosphate backbone of the guide RNA is altered. The modified gRNA can include one or more phosphorothioate, phosphoramidate (e.g., N3′-P5′-phosphoramidate (NP)), 2′-O-methoxy-ethyl (2′MOE), 2′-O-methyl-ethyl (2′ME), and/or methylphosphonate linkages.

In particular embodiments, one or more of the modified nucleotides of the guide sequence and/or one or more of the modified nucleotides of the scaffold region of the guide RNA include a 2′-O-methyl (M) nucleotide, a 2′-O-methyl 3′-phosphorothioate (MS) nucleotide, a 2′-O-methyl-3′-phosphonoacetate (MP) nucleotide, a 2′-O-methyl 3′thioPACE (MSP) nucleotide, or a combination thereof. In some instances, the guide RNA includes one or more MS nucleotides. In other instances, the guide RNA includes one or more MP/MSP nucleotides. In yet other instances, the guide RNA includes one or more MS nucleotides and one or more MP/MSP nucleotides. In further instances, the guide RNA does not include M nucleotides. In certain instances, the guide RNA includes one or more MS nucleotides and/or one or more MP/MSP nucleotides, and further includes one or more M nucleotides. In certain other instances, MS nucleotides and/or MP/MSP nucleotides are the only modified nucleotides present in the guide RNA.

In some aspects, the modified guide RNA, and the Cas proteins (or mRNA encoding the same) described herein may be present in a composition (e.g., a CRISPR/Cas reaction mixture) in particular amounts, ratios, or ranges. For example, a reaction mixture may comprise: a) 1 to 200 pmols of a guide RNA; b) 1 to 100 pmols of a Cas protein, or 0.01 to 3.0 pmols of a DNA or mRNA encoding a Cas protein; c) a guide RNA and a Cas protein, at a molar ratio 0.1:1 to 3:1; and/or d) a guide RNA and a DNA or mRNA encoding the Cas protein, at a molar ratio of 1:1 to 200:1. For example, in some aspects, a reaction mixture comprises a plurality of cells; and i) 1 to 100 pmols of the guide RNA (or pegRNA) per 100,000 cells, and/or ii) 1 to 50 pmols of the Cas protein or 0.01 to 3.0 pmols of the DNA or mRNA encoding the Cas protein, per 100,000 cells. Similarly, in some aspects, a reaction mixture may comprise at least, about, or at most 10, 20, 30, 40, 50, 60, 70, 80, 90, 100, 110, 120, 130, 140, 150, 160, 170, 180, 190, or 200 pmols of the guide RNA, or an amount within a ranged bounded by any combination of the foregoing values, per 1 pmol of the DNA or mRNA encoding the Cas protein. In some aspects, the molar ratio of the guide RNA to the DNA or mRNA encoding the Cas protein is at least, about, or at most 200:1, 190:1, 180:1, 170:1, 160:1, 150:1, 140:1, 130:1, 120:1, 110:1, 100:1, 90:1, 80:1, 70:1, 60:1, 50:1, 40:1, 30:1, 20:1, or 10:1, or a ratio within a range bounded by any combination of the foregoing ratios. In some aspects, a reaction mixture according to the disclosure comprises at least, about, or at most 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or 3.0 pmols of the guide RNA, or an amount within a range bounded by any combination of the foregoing values, per 1 pmol of the Cas protein.

It should be noted that any of the modifications described herein may be combined and incorporated in the guide sequence and/or the scaffold region of the modified gRNA.

In some cases, the guide RNA also includes a structural modification such as a stem loop, e.g., MS2 stem loop or tetraloop.

The guide RNA can be synthesized by any method known to one of ordinary skill in the art. Modified gRNAs can be synthesized using 2′-O-thionocarbamate-protected nucleoside phosphoramidites. Methods are described in, e.g., Dellinger et al., J. American Chemical Society 133, 11540-11556 (2011); Threlfall et al., Organic & Biomolecular Chemistry 10, 746-754 (2012); and Dellinger et al., J American Chemical Society 125, 940-950 (2003).

The chemically modified gRNAs or pegRNAs can be used with any CRISPR-associated technology, e.g., and RNA-guided technology. As described herein, the guide RNA can serve as a guide for any Cas protein or variant or fragment thereof, including any engineered or man-made Cas9 polypeptide. The modified gRNAs or pegRNAs can target DNA and/or RNA molecules in isolated primary cells for ex vivo therapy or in vivo (e.g., in an animal). The methods disclosed herein can be applied to genome editing, gene regulation, imaging, and any other CRISPR-based applications.

### 3. Donor Repair Template

In some embodiments, the present disclosure provides a recombinant donor repair template comprising two homology arms that are homologous to portions of a target DNA sequence (e.g., target gene or locus) at either side of a Cas protein (e.g., Cas9 nuclease) cleavage site. In certain instances, the recombinant donor repair template comprises a reporter cassette that includes a nucleotide sequence encoding a reporter polypeptide (e.g., a detectable polypeptide, fluorescent polypeptide, or a selectable marker), and two homology arms that flank the reporter cassette and are homologous to portions of the target DNA at either side of the Cas protein cleavage site. The reporter cassette can further comprise a sequence encoding a self-cleavage peptide, one or more nuclear localization signals, and/or a fluorescent polypeptide, e.g. superfolder GFP (sfGFP).

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb.

The donor repair template can be cloned into an expression vector. Conventional viral and non-viral based expression vectors known to those of ordinary skill in the art can be used.

In place of a recombinant donor repair template, a single-stranded oligodeoxynucleotide (ssODN) donor template can be used for homologous recombination-mediated repair. An ssODN is useful for introducing short modifications within a target DNA. For instance, ssODN are suited for precisely correcting genetic mutations such as SNPs. ssODNs can contain two flanking, homologous sequences on each side of the target site of Cas protein cleavage and can be oriented in the sense or antisense direction relative to the target DNA. Each flanking sequence can be at least about 10 base pairs (bp), e.g., at least about 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1 kb, 2 kb, 4 kb, or longer. In some embodiments, each homology arm is about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb. The ssODN can be at least about 25 nucleotides (nt) in length, e.g., at least about 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, 100 nt, 150 nt, 200 nt, 250 nt, 300 nt, or longer. In some embodiments, the ssODN is about 25 to about 50; about 50 to about 100; about 100 to about 150; about 150 to about 200; about 200 to about 250; about 250 to about 300; or about 25 nt to about 300 nt in length.

In some embodiments, the ssODN template comprises at least one, e.g., 1, 2, 3, 4, 5, 6, 7, 8, 9, 10, 20, 30, 40, 50, 60, 70, 80, 90, 100, 200, 300, 400, 500, or more modified nucleotides described herein. In some instances, at least 1%, 5%, 10%, 20%, 30%, 40%, 50%, 60%, 70%, 80%, 90%, or 99% of the sequence of the ssODN includes a modified nucleotide. In some embodiments, the modified nucleotides are located at one or both of the terminal ends of the ssODN. The modified nucleotides can be at the first, second, third, fourth, fifth, sixth, seventh, eighth, ninth, or tenth terminal nucleotide, or any combination thereof. For instance, the modified nucleotides can be at the three terminal nucleotides at both ends of the ssODN template. Additionally, the modified nucleotides can be located internal to the terminal ends.

In some aspects, e.g., prime editing, an exogenous DNA repair template is not required. For example, the modified pegRNAs described herein include a reverse transcriptase sequence (e.g., at the 3′ end in proximity to a primer binding site sequence) containing one or more edits to a target nucleic acid, which is used as a template by a prime editor Cas protein when performing prime editing of the target nucleic acid.

### 4. Target DNA

In the CRISPR/Cas system, the target DNA sequence can be immediately followed by a protospacer adjacent motif (PAM) sequence. The target DNA site may lie immediately 5′ of a PAM sequence that is specific to the bacterial species of the Cas protein used. For instance, the PAM sequence of Streptococcus pyogenes-derived Cas9 is NGG; the PAM sequence of Neisseria meningitidis-derived Cas9 is NNNNGATT; the PAM sequence of Streptococcus thermophilus-derived Cas9 is NNAGAA; and the PAM sequence of Treponema denticola-derived Cas9 is NAAAAC. In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system, the selected target DNA sequence should immediately precede (e.g., be located 5′) a 5′NGG PAM, wherein N is any nucleotide, such that the guide sequence of the DNA-targeting RNA (e.g., modified gRNA) base pairs with the opposite strand to mediate cleavage at about 3 base pairs upstream of the PAM sequence.

In some embodiments, the degree of complementarity between a guide sequence of the DNA-targeting RNA (e.g., guide RNA) and its corresponding target DNA sequence, when optimally aligned using a suitable alignment algorithm, is about or more than about 50%, 55%, 60%, 65%, 70%, 75%, 80%, 85%, 90%, 91%, 92%, 93%, 94%, 95%, 96%, 97%, 98%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, Selangor, Malaysia), and ELAND (Illumina, San Diego, Calif).

The target DNA site can be selected in a predefined genomic sequence (gene) using web-based software such as ZiFiT Targeter software (Sander et al., 2007, Nucleic Acids Res, 35:599-605; Sander et al., 2010, Nucleic Acids Res, 38:462-468), E-CRISP (Heigwer et al., 2014, Nat Methods, 11:122-123), RGEN Tools (Bae et al., 2014, Bioinformatics, 30(10):1473-1475), CasFinder (Aach et al., 2014, bioRxiv), DNA2.0 gNRA Design Tool (DNA2.0, Menlo Park, Calif), and the CRISPick Design Tool (Broad Institute, Cambridge, Mass.). Such tools analyze a genomic sequence (e.g., gene or locus of interest) and identify suitable target site for gene editing. To assess off-target gene modifications for each DNA-targeting RNA (e.g., modified gRNA), computationally predictions of off-target sites are made based on quantitative specificity analysis of base-pairing mismatch identity, position and distribution.

### 5. Modulating Gene Expression

The CRISPR/Cas system may be used to regulate gene expression, such as inhibiting gene expression or activating gene expression. As a non-limiting example, a complex comprising a Cas9 variant or fragment and an gRNA that can bind to a target DNA sequence can block or hinder transcription initiation and/elongation by RNA polymerase. This, in turn, can inhibit or repress gene expression of the target DNA. Alternatively, a complex comprising a different Cas9 variant or fragment and an gRNA that can bind to a target DNA sequence can induce or activate gene expression of the target DNA.

Detailed descriptions of methods for performing CRISPR interference (CRISPRi) to inactivate or reduce gene expression can be found in, e.g., Larson et al., Nature Protocols, 2013, 8(11):2180-2196, and Qi et al., Cell, 152, 2013, 1173-1183. In CRISPRi, the gRNA-Cas9 variant complex can bind to a nontemplate strand of a protein-coding region and block transcription elongation. In some cases, when the gRNA-Cas9 variant complex binds to a promoter region of a gene, the complex prevents or hinders transcription initiation.

Detailed descriptions of methods for performing CRISPR activation to increase gene expression can be found in, e.g., Cheng et al., Cell Research, 2013, 23:1163-1171, Konerman et al., Nature, 2015, 517:583-588, and U.S. Pat. No. 8,697,359.

For CRISPR-based control of gene expression, a catalytically inactive variant of the Cas protein (e.g., Cas9 polypeptide) that lacks endonucleolytic activity can be used. In some embodiments, the Cas protein is a Cas9 variant that contains at least two point mutations in the RuvC-like and HNH nuclease domains. In some embodiments, the Cas9 variant has D10A and H840A amino acid substitutions, which is referred to as dCas9 (Jinek et al., Science, 2012, 337:816-821; Qi et al., Cell, 152(5):1173-1183). In some cases, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Application Pub. No. WO2013/176772. The dCas9 enzyme can contain a mutation at D10, E762, H983 or D986, as well as a mutation at H840 or N863. In some cases, the dCas9 enzyme contains a D1OA or DION mutation. Also, the dCas9 enzyme can include a H840A, H840Y, or H840N. In some cases, the dCas9 enzyme comprises D1OA and H840A; D1OA and H840Y; D1OA and H840N; D1ON and H840A; D1ON and H840Y; or D1ON and H840N substitutions. The substitutions can be conservative or non-conservative substitutions to render the Cas9 polypeptide catalytically inactive and able to bind to target DNA.

In certain embodiments, the dCas9 polypeptide is catalytically inactive such as defective in nuclease activity. In some instances, the dCas9 enzyme or a variant or fragment thereof can block transcription of a target sequence, and in some cases, block RNA polymerase. In other instances, the dCas9 enzyme or a variant or fragment thereof can activate transcription of a target sequence.

In certain embodiments, the Cas9 variant lacking endonucleolytic activity (e.g., dCas9) can be fused to a transcriptional repression domain, e.g., a Kruppel associated box (KRAB) domain, or a transcriptional activation domain, e.g., a VP16 transactivation domain. In some embodiments, the Cas9 variant is a fusion polypeptide comprising dCas9 and a transcription factor, e.g., RNA polymerase omega factor, heat shock factor 1, or a fragment thereof. In other embodiments, the Cas9 variant is a fusion polypeptide comprising dCas9 and a DNA methylase, histone acetylase, or a fragment thereof.

For CRISPR-based control of gene expression mediated by RNA binding and/or RNA cleavage, a suitable Cas protein (e.g., Cas9 polypeptide) variant having endoribonuclease activity, as described in, e.g., O'Connell et al., Nature, 2014, 516:263-266, can be used. Other useful Cas protein (e.g., Cas9) variants are described in, e.g., U.S. Pat. No. 9,745,610. Other CRISPR-related enzymes that can cleave RNA include a Csy4 endoribonuclease, a CRISPR-related Cas6 enzyme, a Cas5 family member enzyme, a Cas6 family member enzyme, a Type I CRISPR system endoribonuclease, a Type II CRISPR system endoribonuclease, a Type III CRISPR system endoribonuclease, and variants thereof.

In some embodiments of CRISPR-based RNA cleavage, a DNA oligonucleotide containing a PAM sequence (e.g., PAMmer) is used with the modified gRNA and Cas protein (e.g., Cas9) variant described herein to bind to and cleave a single-stranded RNA transcript. Detailed descriptions of suitable PAMmer sequences are found in, e.g., O'Connell et al., Nature, 2014, 516:263-266.

In some embodiments, a plurality of modified gRNAs and/or pegRNAs is used to target different regions of a target gene to regulate gene expression of that target gene. The plurality of modified gRNAs and/or pegRNAs can provide synergistic modulation (e.g., inhibition or activation) of gene expression of a single target gene compared to each modified gRNA alone. In other embodiments, a plurality of modified gRNAs/pegRNAs is used to regulate gene expression of at least two different target genes.

### B. Cells Ex Vivo

In some aspects of the present methods, the target sequence is in a cell. The present methods can be used to edit, modulate, cleave, nick, or bind a target sequence in a nucleic acid in any cell of interest, including primary cells, immortalized cells, cells from cell lines, cells from cell culture, and others. In some embodiments, the cell is a cell type with one or challenging conditions. For example, cells having high nuclease (e.g. ribonuclease, exonuclease, exoribonuclease) expression, concentration and/or activity, for example, cell types high in a particular nuclease.

The compositions and methods disclosed herein can be used to edit or regulate the expression of a target nucleic acid in a primary cell of interest. The primary cell can be a cell isolated from any multicellular organism, e.g., a plant cell (e.g., a rice cell, a wheat cell, a tomato cell, an Arabidopsis thaliana cell, a Zea mays cell, and the like), a cell from a multicellular protist, a cell from a multicellular fungus, an animal cell such as a cell from an invertebrate animal (e.g., fruit fly, cnidarian, echinoderm, nematode, etc.) or a cell from a vertebrate animal (e.g., fish, amphibian, reptile, bird, mammal, etc.), a cell from a human, a cell from a healthy human, a cell from a human patient, a cell from a cancer patient, etc. In some cases, the primary cell with genome edits or induced gene regulation can be transplanted to a subject (e.g., patient). For instance, the primary cell can be derived from the subject (e.g., patient) to be treated.

Any type of primary cell may be of interest, such as a stem cell, e.g., embryonic stem cell, induced pluripotent stem cell, adult stem cell (e.g., mesenchymal stem cell, neural stem cell, hematopoietic stem cell, organ stem cell), a progenitor cell, a somatic cell (e.g., fibroblast, hepatocyte, heart cell, liver cell, pancreatic cell, muscle cell, skin cell, blood cell, neural cell, immune cell), and any other cell of the body, e.g., human body. Primary cells are typically derived from a subject, e.g., an animal subject or a human subject, and allowed to grow in vitro for a limited number of passages. In some embodiments, the cells are disease cells or derived from a subject with a disease. For instance, the cells can be cancer or tumor cells.

Primary cells can be harvested from a subject by any standard method. For instance, cells from tissues, such as skin, muscle, bone marrow, spleen, liver, kidney, pancreas, lung, intestine, stomach, etc., can be harvested by a tissue biopsy or a fine needle aspirate. Blood cells and/or immune cells can be isolated from whole blood, plasma or serum. In some cases, suitable primary cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem and progenitor cell (HSPC) such as CD34+ HSPCs, or a non-pluripotent stem cell. In some cases, the cell can be any immune cell including, but not limited to, any T cell such as tumor infiltrating cells (TILs), CD3+ T cells, CD4+ T cells, CD8+ T cells, or any other type of T cell. The T cell can also include memory T cells, memory stem T cells, or effector T cells. The T cells can also be skewed towards particular populations and phenotypes. For example, the T cells can be skewed to phenotypically comprise CD45RO(−), CCR7(+), CD45RA(+), CD62L(+), CD27(+), CD28(+) and/or IL-7Ra(+). Suitable cells can be selected that comprise one of more markers selected from a list comprising CD45RO(−), CCR7(+), CD45RA(+), CD62L(+), CD27(+), CD28(+) and/or IL-7Ra(+). Induced pluripotent stem cells can be generated from differentiated cells according to standard protocols described in, for example, U.S. Pat. Nos. 7,682,828, 8,058,065, 8,530,238, 8,871,504, 8,900,871 and 8,791,248.

### C. Ex Vivo Therapy

The methods described herein can be used in ex vivo therapy. Ex vivo therapy can comprise administering a composition (e.g., a cell) generated or modified outside of an organism to a subject (e.g., patient). In some embodiments, the composition (e.g. comprising a cell) can be generated or modified by the methods disclosed herein. For example, ex vivo therapy can comprise administering a primary cell generated or modified outside of an organism to a subject (e.g., patient), wherein the primary cell has been cultured and edited/modulated in vitro in accordance with the methods of the present disclosure that includes contacting the target nucleic acid in the primary cell with one or more modified gRNAs described herein and a Cas protein (e.g., Cas9 polypeptide) or variant or fragment thereof, an mRNA encoding a Cas protein (e.g., Cas9 polypeptide) or variant or fragment thereof, or a recombinant expression vector comprising a nucleotide sequence encoding a Cas protein (e.g., Cas9 polypeptide) or variant or fragment thereof.

In some embodiments, the composition (e.g., a cell) can be derived from the subject (e.g., patient) to be treated by ex vivo therapy. In some embodiments, ex vivo therapy can include cell-based therapy, such as adoptive immunotherapy.

In some embodiments, the composition used in ex vivo therapy can be a cell. The cell can be a primary cell, including but not limited to, peripheral blood mononuclear cells (PBMCs), peripheral blood lymphocytes (PBLs), and other blood cell subsets. The primary cell can be an immune cell. The primary cell can be a T cell (e.g., CD3+ T cells, CD4+ T cells, and/or CD8+ T cells), a natural killer cell, a monocyte, a natural killer T cell, a monocyte-precursor cell, a hematopoietic stem cell or a non-pluripotent stem cell, a stem cell, or a progenitor cell. The primary cell can be a hematopoietic stem or progenitor cell (HSPC) such as CD34+ HSPCs. The primary cell can be a human cell. The primary cell can be isolated, selected, and/or cultured. The primary cell can be expanded ex vivo. The primary cell can be expanded in vivo. The primary cell can be CD45RO(−), CCR7(+), CD45RA(+), CD62L(+), CD27(+), CD28(+), and/or IL-7Ra(+). The primary cell can be autologous to a subject receiving the cell. Or the primary cell can be non-autologous to the subject. The primary cell can be a good manufacturing practices (GMP) compatible reagent. The primary cell can be a part of a combination therapy to treat diseases, including cancer, infections, autoimmune disorders, or graft-versus-host disease (GVHD), in a subject having or at risk for such diseases.

As a non-limiting example of ex vivo therapy, a primary cell can be isolated from a multicellular organism (e.g., a plant, multicellular protist, multicellular fungus, invertebrate animal, vertebrate animal such as human, etc.) prior to contacting a target nucleic acid within the primary cell with a Cas protein and a modified gRNA. After contacting the target nucleic acid with the Cas protein and the guide RNA, the primary cell or its progeny (e.g., a cell derived from the primary cell) can be returned to the multicellular organism.

In some embodiments, the Cas protein and the guide RNA are introduced into a living organism, such as by introduction to a serum-containing fluid in or from the living organism (e.g., whole blood, plasma or serum).

D. Methods for Introducing Nucleic Acids and/or Polypeptides Into Target Cells

Methods for introducing polypeptides and nucleic acids into a target cell (host cell) are known in the art and can be employed in the present methods, to introduce a nucleic acid (e.g., a nucleotide sequence encoding a Cas protein, a modified guide RNA, a donor repair template for homology-directed repair (HDR), etc.), a polypeptide (such as a Cas protein, a polymerase, a deaminase, etc.), or an RNP (e.g. gRNA/Cas protein complex) into a cell, e.g., a primary cell such as a stem cell, a progenitor cell, or a differentiated cell. Non-limiting examples of suitable methods include electroporation, viral or bacteriophage infection, transfection, microinjection, conjugation, protoplast fusion, lipofection, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated delivery (e.g. lipid nanoparticle-mediated delivery, polymer nanoparticle-mediated delivery, hybrid lipid-polymer nanoparticle mediated delivery), and the like.

In some embodiments, the components of a CRISPR system can be introduced into a cell using a delivery system. In certain instances, the delivery system comprises a nanoparticle, a microparticle (e.g., a polymer micropolymer), a liposome, a micelle, a virosome, a viral particle, a virus-like particle (VLP), a nucleic acid complex, a transfection agent, an electroporation agent (e.g., using a NEON transfection system), a nucleofection agent, a lipofection agent, and/or a buffer system that includes the component(s) to be delivered. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996. See Advanced Drug Delivery Reviews 2021, Volume 168, for reviews on preparation of nanoparticles such as lipid, polymer or hybrid lipid-polymer nanoparticles.

### E. Methods for Assessing the Efficiency of Genome Editing

To functionally test the presence of the correct genomic editing modification, the target DNA can be analyzed by standard methods known to those in the art. For example, indel mutations can be identified by sequencing using the SURVEYOR® mutation detection kit (Integrated DNA Technologies, Coralville, Iowa) or the Guide-it™ Indel Identification Kit (Clontech, Mountain View, Calif). Homology-directed repair (HDR), base editing, or prime editing-mediated edits can be detected by PCR-based methods, and in combination with sequencing or RFLP analysis. Non-limiting examples of PCR-based kits include the Guide-it™ Mutation Detection Kit (Clontech) and the GeneArt® Genomic Cleavage Detection Kit (Life Technologies, Carlsbad, Calif). Deep sequencing can also be used, particularly for a large number of samples or potential target/off-target sites.

In certain embodiments, the efficiency (e.g., specificity) of genome editing corresponds to the number or percentage of on-target genome editing events relative to the number or percentage of all genome editing events, including on-target and off-target events. In some embodiments, the efficiency of editing of a target region corresponds to the number of expected editing of that target region, at the level of either single cells or cell populations.

In some embodiments, the modified gRNAs described herein are capable of enhancing genome editing of a target DNA sequence in a cell such as a primary cell relative to the corresponding unmodified gRNAs. The genome editing can comprise homology-directed repair (HDR) (e.g., insertions, deletions, or point mutations), prime editing, base editing, or nonhomologous end joining (NHEJ).

In certain embodiments, the nuclease-mediated genome editing efficiency of a target DNA sequence in a cell is enhanced by at least about 5%, 10%, 15%, 20%, 25%, 30%, 35%, 40%, 45%, 0.5-fold, 0.6-fold, 0.7-fold, 0.8-fold, 0.9-fold, 1-fold, 1.1-fold, 1.2-fold, 1.3-fold, 1.4-fold, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 15-fold, 20-fold, 25-fold, 30-fold, 35-fold, 40-fold, 45-fold, 50-fold, or greater in the presence of a guide RNA described herein compared to the corresponding unmodified gRNA sequence. In some other embodiments, the efficiency is compared to a corresponding gRNA with different modifications and achieves a level of enhancement described above. For example, gRNAs with 1×, 2×, or 3×MS at the 5′ end as well as 2×, 3×, or 4×MP or MSP at the 3′ end, may be compared to gRNAs with the same number of MS instead of MP/MSP (i.e. 1×, 2×, or 3×MS at the 5′ end as well as 2×, 3×, or 4×MS at the 3′ end).

### F. Methods for Preventing or Treating a Genetic Disease in a Subject

The modified gRNAs can be applied to targeted nuclease-based therapeutics of genetic diseases. Current approaches for precisely correcting genetic mutations in the genome of primary patient cells can be very inefficient (sometimes less than 1% of cells can be precisely edited). The modified gRNAs described herein can enhance the activity of genome editing and increase the efficacy of genome editing-based therapies. In particular embodiments, modified gRNAs may be used for in vivo gene editing of genes in subjects with a genetic disease. The modified gRNAs can be administered to a subject via any suitable route of administration and at doses or amounts sufficient to enhance the effect (e.g., improve the genome editing efficiency) of the nuclease-based therapy.

Provided herein is a method for preventing or treating a genetic disease in a subject in need thereof by correcting a genetic mutation associated with the disease. The method includes administering to the subject a modified guide RNA described herein in an amount that is sufficient to correct the mutation. Also provided herein is the use of a modified guide RNA described herein in the manufacture of a medicament for preventing or treating a genetic disease in a subject in need thereof by correcting a genetic mutation associated with the disease. The modified guide RNA can be contained in a composition that also includes a Cas protein (e.g., Cas9 polypeptide), an mRNA encoding a Cas protein, or a recombinant expression vector comprising a nucleotide sequence encoding a Cas protein. In some instances, the modified guide RNA is included in a delivery system described above.

The genetic diseases that may be corrected by the method include, but are not limited to, X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation disease or disorders, inflammation, immune-related diseases or disorders, metabolic diseases, liver diseases and disorders, kidney diseases and disorders, muscular/skeletal diseases and disorders (e.g., muscular dystrophy, Duchenne muscular dystrophy), neurological and neuronal diseases and disorders, cardiovascular diseases and disorders, pulmonary diseases and disorders, ocular diseases and disorders, viral infections (e.g., HIV infection), and the like.

### EXAMPLES

Aspects of the present teachings can be further understood in light of the following examples, which should not be construed as limiting the scope of the present teachings in any way.

Various general methods and reagents were used in the examples which follow, and are described below to facilitate understanding of the examples, though it should be understood that variations and alternatives in preparation, testing and other details may be employed in accordance with the teachings herein.

Preparation of RNAs and mRNAs.

RNA oligomers were synthesized on Dr. Oligo™ 48 and 96 synthesizers (Biolytic Lab Performance Inc.) using 2′-O-thionocarbamate-protected nucleoside phosphoramidites (Sigma-Aldrich and Hongene) on controlled pore glass (LGC) according to previously described procedures. The 2′-O-methyl-3′-O-(diisopropylamino)-phosphinoacetic acid-1,1-dimethylcyanoethyl ester-5′-O-dimethoxytrityl nucleosides used for synthesis of MP-modified RNAs were purchased from Glen Research and Hongene. For phosphorothioate containing oligomers, the iodine oxidation step after the coupling reaction was replaced by a sulfurization step using a 0.05 M solution of 3-((N,N-dimethylaminomethylidene)amino)-3H-1,2,4-dithiazole-5-thione in a pyridine-acetonitrile (3:2) mixture for 6 min. Unless otherwise noted, reagents for solid-phase RNA synthesis were purchased from Glen Research and Honeywell. The phosphonoacetate modifications incorporated in the MP-modified gRNAs were synthesized using protocols adapted from previous publications (see, e.g., Dellinger et al., 2003 and Threlfall et al., 2012, supra), by using the commercially available protected nucleoside phosphinoamidite monomers above. All oligonucleotides were purified using reversed-phase high-performance liquid chromatography (RP-HPLC) and analyzed by liquid chromatography—mass spectrometry (LC-MS) using an Agilent 1290 Infinity series LC system coupled to an Agilent 6545 Q-TOF (time-of-flight) mass spectrometer. In all cases, the mass determined by deconvolution of the series of peaks comprising multiple charge states in a mass spectrum of purified gRNA matched the expected mass within error of the calibrated instrument (the specification for quality assurance used in this assay is that the observed mass of purified gRNA is within 0.01% of the calculated mass), thus confirming the composition of each synthetic gRNA.

CleanCap® Cas9 mRNA fully substituted with 5-methoxyuridine was purchased from TriLink (L-7206). BE4-Gam mRNA and PE2 mRNA, which encode BE4-Gam protein and PE2 protein respectively, were purchased from TriLink as custom orders by providing the coding sequences to which TriLink added their own proprietary 5′ and 3′ UTRs. The custom mRNAs were fully substituted with 5-methylcytidine and pseudouridine, capped with CleanCap® AG, and polyA tailed.

### Cell Culture and Nucleofections.

Human K562 cells were obtained from ATCC® and cultured in RPMI 1640+GlutaMAX™ media (Gibco™) supplemented with 10% fetal bovine serum (Gibco™). K562 cells (within passage number 4 to 14) were nucleofected using a Lonza 4D-Nucleofector® (96-well shuttle device, program FF-120) per manufacturer's instructions utilizing a Lonza SF Cell Line kit (V4SC-2960) with 0.2 million cells per transfection in 20 μL of SF buffer combined with 6 μL of 125 pmoles of gRNA and 1.87 pmoles of BE4-Gam mRNA in PBS buffer for cytidine base editing or combined with 8 μL of 125 pmoles of pegRNA with 100 pmoles of nicking gRNA and 1.35 pmoles of PE2 mRNA in PBS buffer for prime editing. Cells were cultured at 37° C. in ambient oxygen and 5% carbon dioxide and were harvested at 48 hr post-transfection.

Human Jurkat Clone E6-1 cells were obtained from ATCC® and were cultured in RPMI 1640+GlutaMAX™ media supplemented with 10% fetal bovine serum. Jurkat cells (within passage number 7 to 20) were nucleofected (program CL-120) utilizing a Lonza SE Cell Line kit (V4SC-1960) with 0.2 million cells in 20 μL of SE buffer combined with 8 μL of 125 pmoles of pegRNA, 100 pmoles of nicking gRNA and 1.35 pmoles of PE2 mRNA in PBS buffer. Cultured cells were harvested at 72 hr post-transfection.

Human HepG2 cells were obtained from ATCC® and were cultured in Dulbecco's Modified Eagle's Medium (DMEM)+L-Glutamine+4.5 g/L D-Glucose media (Gibco™) supplemented with 10% fetal bovine serum. HepG2 cells (within passage number 4 to 13) were spun down from culture media and were either rinsed or not with PBS and spun down again. Cells were nucleofected (program EH-100) utilizing a Lonza SF Cell Line kit (V4SC-2960) with 0.2 million cells in 20 μL of SF buffer combined with 3 μL of 10 pmoles of gRNA and 0.0625 pmoles of Cas9 mRNA in PBS buffer, or were nucleofected in the presence of residual serum by combining 0.2 million cells in 20 μL of SF buffer with 5 μL of 30 pmoles of gRNA and 0.5 pmoles of Cas9 mRNA or 12.5 pmoles of S. pyogenes Cas9 (SpCas9) protein (Aldeveron) in PBS buffer. For 163mer gRNAs, 0.2 million cells were likewise nucleofected in the presence of residual serum and SF buffer by combining these with 5 μL of 125 pmoles of 163mer gRNA and 50 pmoles of SpCas9 protein in PBS buffer. For all RNP transfections, gRNA was pre-complexed with SpCas9 protein (Aldevron) in PBS buffer by combining and incubating at room temperature for about 20 min before combining with cells in SF buffer for nucleofection. For mRNA transfections, gRNA was likewise combined with Cas9 mRNA (TriLink) in PBS buffer and kept on ice for about 20 min until combined with cells in SF buffer for nucleofection. Cultured HepG2 cells were harvested at about 72 hr post-transfection.

Human primary T cells (LP, CR, CD3+, NS) were obtained from AllCells (Alameda, CA) and were cultured in RPMI 1640+GlutaMAX™ media supplemented with 10% fetal bovine serum, 5 ng/mL of human IL-7 and 5 ng/mL of human IL-15 (Gibco™). Primary T cells were activated for 48 hr with anti-human CD3/CD28 magnetic Dynabeads™ (Thermo Fisher) at a beads-to-cells concentration of 3:1. Debeaded primary T cells were nucleofected (program EO-115) utilizing a Lonza P3 Primary Cell kit (V4SP-3960) with 0.2 million cells in 20 μL of P3 buffer combined with 2.7 μL of 5 pmoles of gRNA and 0.0625 pmoles of Cas9 mRNA in PBS buffer. Cultured cells were harvested at 7 days post-transfection. Throughout the culture period, T cells were maintained at an approximate density of 1M cells per mL of media. Following electroporation, additional media was added every 2 days.

qRT-PCR Assays.

Human K562 cells were cultured as above, and 0.2 million cells per replicate were nucleofected with 125 pmoles of gRNA (without Cas9 mRNA or protein) as described. For each timepoint, cells were collected in 1.7-mL Eppendorf Tubes®, rinsed with PBS, then resuspended in 750 μL of a lysis reagent, QIAzol™ and kept at room temperature for 5 min before transferring to a −20° C. freezer. Total RNA in PBS was isolated from QIAzol™ plus chloroform extracts using a kit for RNA purification, RNeasy® Micro (Qiagen) on a robotic workstation for nucleic acid purification, QIAcube® HT, and then immediately reverse transcribed using a ProtoScript® II first-strand cDNA synthesis kit (NEB). qRT-PCR was performed on an Applied Biosystems QuantStudio™ 6 Flex instrument using TaqPath™ ProAmp™ master mix with two TaqMan™ MGB probes, one for gRNA labeled with FAM and the other for U6 snRNA labeled with VIC (Thermo Fisher) for normalization to the amount of total RNA isolated, calculated as ΔCt. The ΔCt values for triplicate samples were averaged and normalized to the lowest observed mean ΔCt value to calculate ΔΔCt values. Relative gRNA levels were calculated as 2^{-ΔΔCt}.

### PCR-Targeted Deep Sequencing and Quantification of Targeted Genomic Modifications.

Genomic DNA purification and construction of PCR-targeted deep sequencing libraries were performed as previously described. Library concentration was determined using a Qubit™ dsDNA BR assay kit (Thermo Fisher). Paired-end 2×220-bp reads were sequenced on a next generation sequencer, MiSeq™ (Illumina) at 0.8 ng/μL of PCR-amplified library along with 20.5% PhiX.

Paired-end reads were merged using FLASH version 1.2.11 software and then mapped to the human genome using BWA-MEM software (bwa−0.7.10) set to default parameters. Reads were scored as having an indel or not according to whether an insertion or a deletion was found within 10 bp's of the Cas9 cleavage site. For prime editing analysis, reads were scored as having an edit if the desired edit was identified in the read. For cytidine base editing analysis, reads were scored as base edited if cytidines were edited within a window of 10-20 bp upstream of the PAM site. For each replicate in each experiment, mapped reads were segregated according to mapped amplicon locus and were binned by the presence or absence of an indel or edit. The tally of reads per bin was used to calculate % indels or % edits produced at each locus. Indel or edit yields and standard deviations for plots were calculated by logit transformation of % indels or % edits, transformed as ln(r/(1−r)) where r is % indels or % edits per specific locus, to closely approximate a normal distribution. Triplicate mock transfections provided a mean mock control (or negative control), and triplicate samples showing a mean indel yield or mean edit yield significantly higher (t-test p<0.05) than the corresponding negative control were considered above background.

### Example 1

This example evaluated the stability of guide RNAs having 2′-O-methyl-3′-phosphonoacetate (MP) and 2′-O-methyl-3′-phosphorothioate (MS) modifications at their 3′ ends. To evaluate the relative lifetimes of single-guide RNAs with MS or MP modifications at the 3′ end in transfected cells, guide RNAs were synthesized with MS modifications at the first three internucleotide linkages at the 5′ end and either MS modifications at the last three internucleotide linkages at the 3′ end (denoted as 3×MS, 3×MS) or 2, 3 or 4 consecutive MP modifications at the terminal internucleotide linkages at the 3′ end (denoted as 3×MS, 2×MP; 3×MS, 3×MP; and 3×MS, 4×MP; respectively). Each modified gRNA was transfected individually into human K562 cells in the absence of Cas9, and qRT-PCR was used to measure the relative amount of sgRNA remaining in cells collected at a series of timepoints from 1 to 96 hours post-transfection.

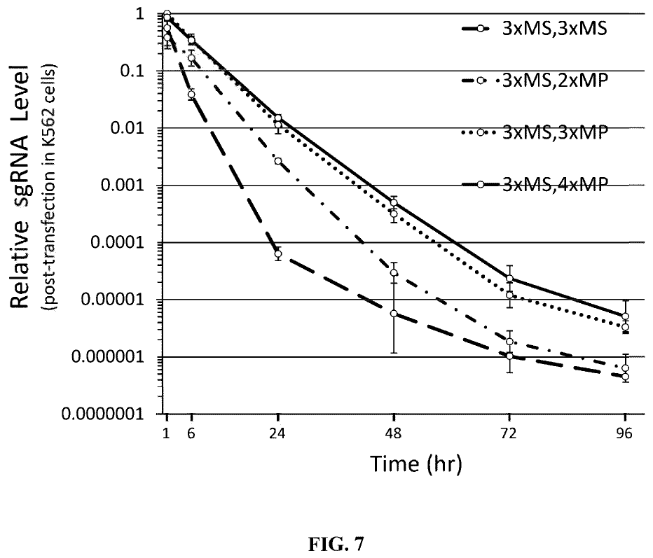

As shown by FIG. 7, a much steeper decline in the relative level of the 3×MS, 3×MS gRNA detected across 1, 6, and 24 h post-transfection was observed, in comparison to that for any of the gRNAs modified with MPs at the 3′ end (either two, three, or four consecutive MPs). Specifically, at 1 h post-transfection, the relative amounts of transfected gRNA differed by only 2.6-fold with largely overlapping error bars among all four variations of 3′ end protection, whereas much larger differences were observed at 6 h post-transfection, when the remaining amount of 3×MS, 3×MS-protected gRNA had dropped to a relative level of about 1/10 (0.039) that of the 3×MS, 3×MP- and 3×MS, 4×MP-protected gRNAs (0.341-0.351). The differences became even larger at the 24 h time point where they varied according to the level of 3′ end protection in a logical progression from having 3×MS to 2×MP to 3×MP to 4×MP at the 3′ end, resulting in residual gRNA levels that spanned ca. 250-fold, consistent with the level of 3′ end protection. Thus, it was found that incorporating MP modifications at the 3′ end of uncomplexed gRNAs can significantly enhance their stability in transfected cells relative to MS modifications, specifically by 1-2 orders of magnitude for three different MP-modified gRNAs tested in parallel with an MS-only modified gRNA. The designs with three or four consecutive MPs at the 3′ end can prolong the lifetimes of the free gRNAs across even longer time points (72 and 96 h post-transfection).

### Example 2

Phosphonate modifications can be stably incorporated in DNA and RNA oligonucleotides and have been demonstrated to increase their resistance to nucleases relative to phosphorothioates. In a previous report exploring the use of MP to enhance the specificity of gRNAs by incorporating it in the 20-nt guide sequence portion, it was found that MP at specific sequence positions such as position 5 or 11 (counted from the 5′ end of the 20 nucleotides) can significantly reduce off-target editing while maintaining high on-target editing as described in, e.g., Ryan et al., Nucleic Acids Research 46, 792-803 (2018). However, it was also reported that incorporating MP modifications within the first one, two or three nucleotides at the 5′ ends of gRNAs can, in some guide sequences, decrease their on-target cleavage activity and/or increase their off-target activities and thus lower specificity (see, e.g., Ryan et al., 2018).

To further explore the potential utility of phosphonate modifications in guide RNAs, the performance of gRNAs containing different numbers of consecutive 2′-O-methyl-3′-phosphonoacetate (2′-O-methyl-3′-PACE, or “MP”) modifications at the 3′ end was evaluated in comparison to guide RNAs with 2′-O-methyl-3′-phosphorothioate (or “MS”) modifications at that end. The results of this study are further described in Ryan et al. “Phosphonoacetate Modifications Enhance the Stability and Editing Yields of Guide RNAs for Cas9 Editors.” Biochemistry (2022) doi.org/10.1021/acs.biochem.1c00768.

This experiment was designed to evaluate Cas activity following co-transfection of HepG2 cells with relatively low (sub-saturating) amounts of chemically-modified guide RNA and an mRNA encoding a Cas protein, using HBB as the target gene. Such sub-saturating amounts constitute challenging conditions for editing a target region of the cell.

For three sets of samples, an mRNA encoding Cas9 was co-transfected into human hepatocytes (HepG2 cells) with modified gRNAs targeting HBB. (see Table 1 supra). For a fourth set of HepG2 cells, modified gRNAs targeting the same site in HBB were precomplexed with purified recombinant Cas9 protein to form RNPs, which were then transfected into the cells. Each transfection was performed in triplicate samples of cells that were cultured separately. Genomic DNA was harvested, the HBB target and off-target sequence were amplified using primers specific for the HBB gene and an intergenic off-target site, respectively, to produce amplicons that were sequenced, and the extent of editing (“% Indels”) at the target and off-target sites was determined from the sequencing results. ON and OFF indicate the on-target and off-target sequences, respectively. The intergenic off-target locus was monitored because it is known to suffer high collateral activity when targeting the chosen target sequence in the HBB gene. Editing yields for the modified gRNA described in Table 1 are plotted as bar graphs in FIGS. 2-5.

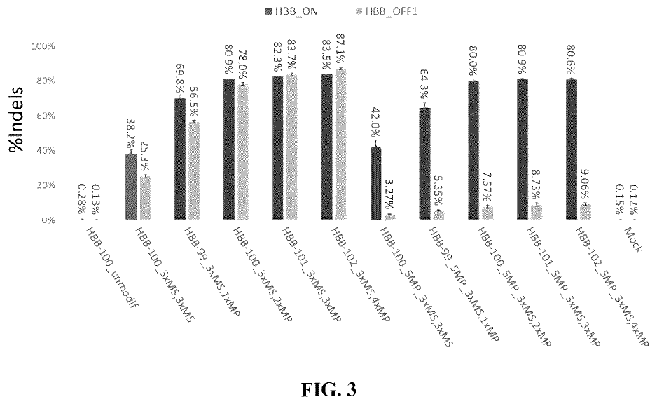

As demonstrated by FIGS. 2-5, co-transfection with sub-saturating levels of a modified guide RNA targeting HBB and an mRNA encoding a Cas protein (or an RNP complex of the modified guide RNAs) resulted in a higher level of editing yield relative to samples co-transfected with an equivalent amount of an unmodified gRNA. Furthermore, the addition of 2, 3, or 4 MP modifications at the 3′ end of the modified gRNA resulted in a progressively higher increase in editing yield, in addition to a substantial increase relative to modified gRNAs comprising 3 MS modifications at the 3′ end of the modified gRNA. See, e.g., FIGS. 2 and 4. As shown by FIGS. 3 and 5, the inclusion of an MP modification at position 5 or 11 (counted from the 5′ end of the 20-nt guide sequence in the gRNA) also reduced off-target activity. In particular, the inclusion of an MP at the position 5 in the gRNA had minimal impact on editing yield while substantially reducing off-target activity.

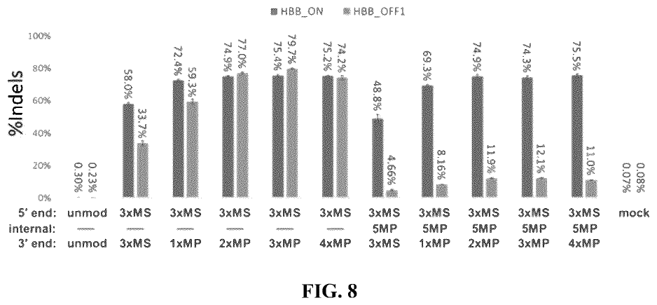

It was further observed that MP modification of the 3′ end significantly enhanced editing yields in HepG2 cells (FIG. 3). For instance, designs with 2, 3 or 4 consecutive MP modifications at the 3′ end gave at least 2-fold more Cas9-mediated indels than a comparable design with 3×MS at the 3′ end (81%-83% at the ON-target site for the 2×MP, 3×MP and 4×MP modifications versus 38% for 3×MS at the 3′ end). A similar trend was observed for the same gRNAs transfected into primary human T cells although the increase was more modest in that the 2×MP, 3×MP and 4×MP modifications gave a 1.3-fold higher level of ON-target indels than when using 3×MS at the 3′ end (FIG. 8). Incorporation of an additional MP at position 5 in the 20-nt guide sequence portion of the gRNA significantly lowered editing at the OFF1 site in both cell types while maintaining high on-target editing efficiency, as previously reported as a means for enhancing specificity (see, e.g., Ryan et al., 2018). Indeed, indels at the OFF1 site were reduced by 7-10 fold in HepG2 cells by incorporating MP at position 5 and similarly by 6-7 fold in primary T cells.

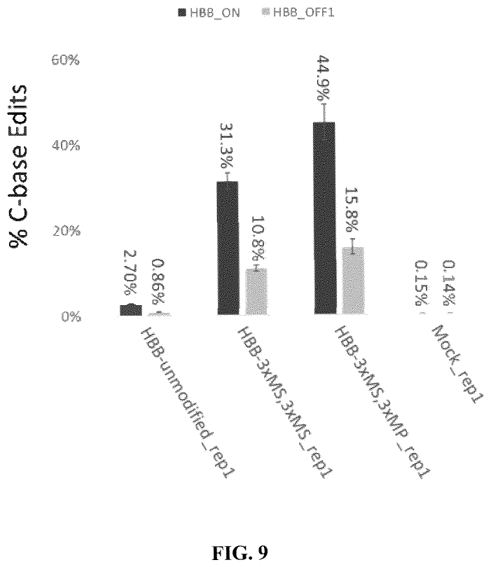

As shown by FIG. 9, the use of chemically-modified gRNAs was also evaluated in combination with a base editor. Base editors are a class of alternative genome editing systems built around Cas9 nickase (nCas9) or dead Cas9 (dCas9) fused to one of various deaminases that enable editing of genomic DNA in cells without creating double-stranded breaks. Both cytidine base editors (CBE) and adenosine base editors (ABE) have been reported, and these have inspired a number of variations for base editing. The potential benefits of using MP modifications in contrast to MS modifications at the 3′ ends of such gRNAs were tested in the context of a CBE, namely BE4-Gam mRNA. A 1.4-fold higher level of cytidine editing was observed by using CBE mRNA in K562 cells co-transfected with gRNA modified with MP at the 3′ end versus an alternative design with MS at the 3′ end.

### Example 3

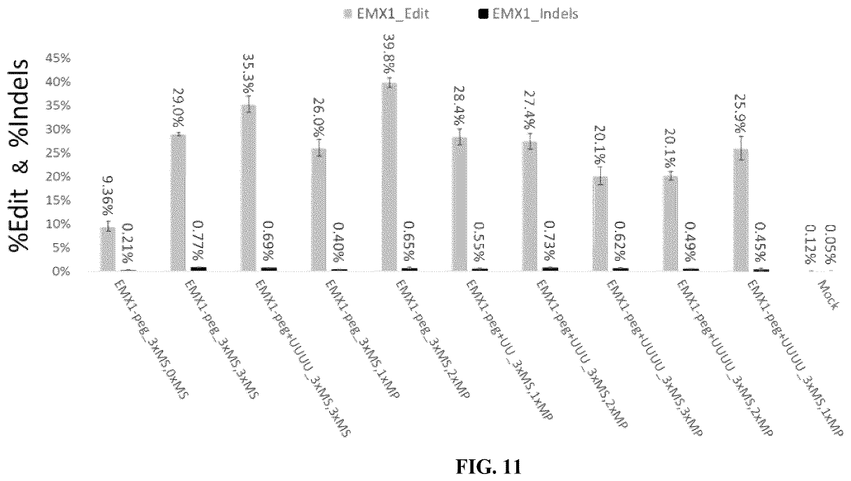

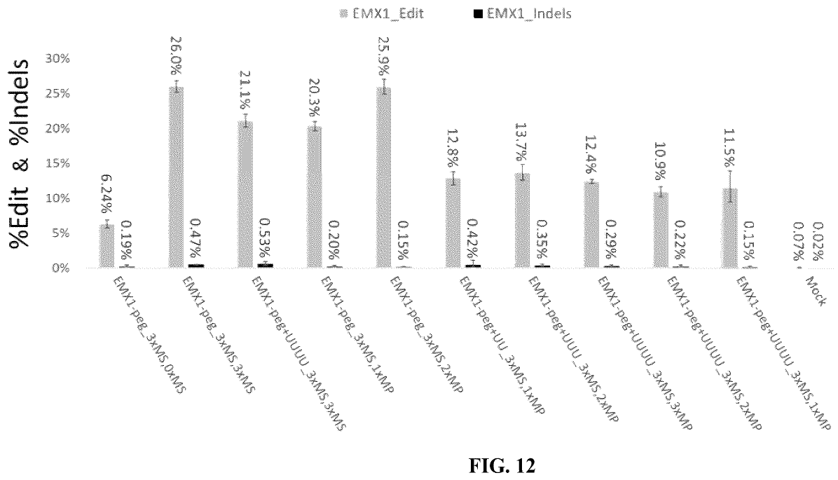

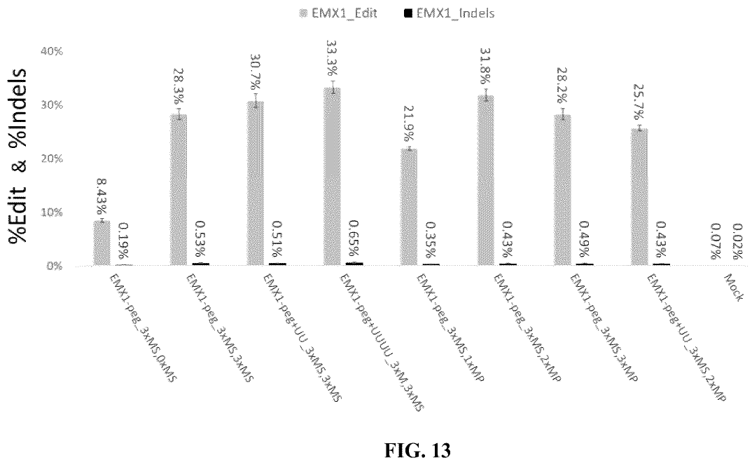

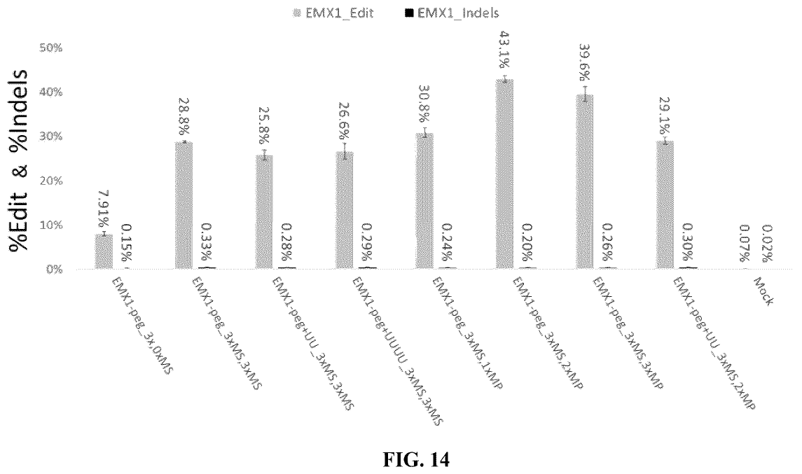

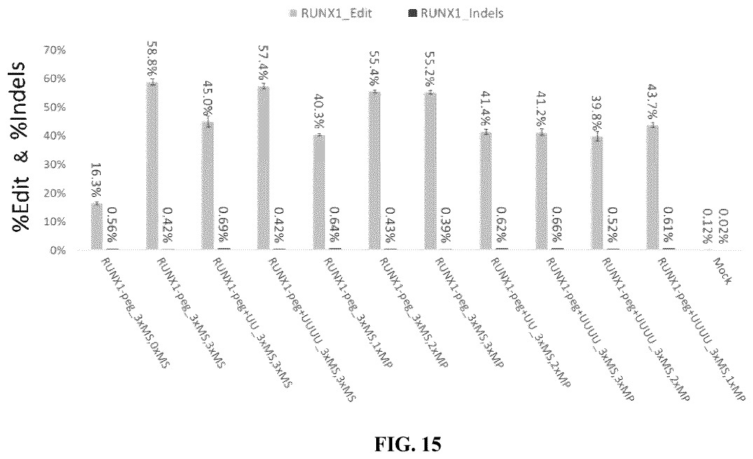

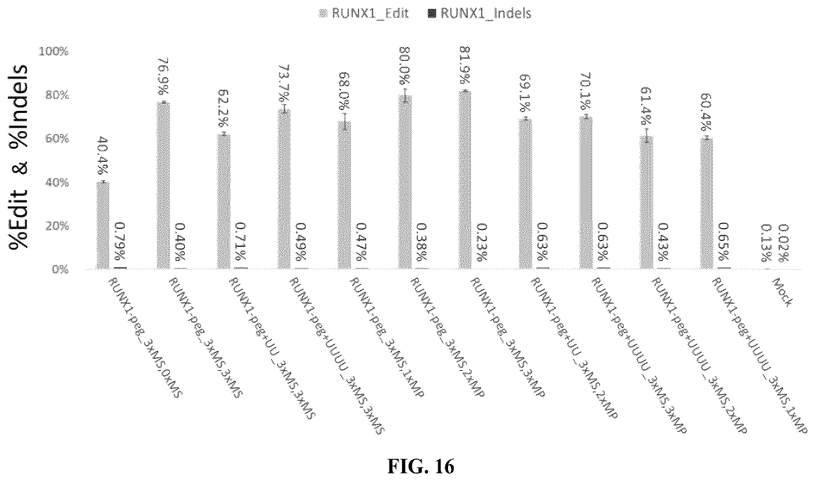

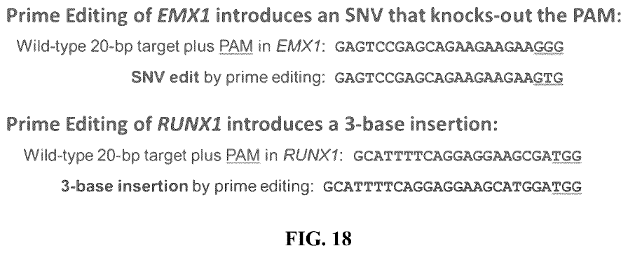

This example evaluated the use of 2′-O-methyl-3′-phosphonoacetate (MP) and 2′-O-methyl-3′-phosphorothioate (MS) modifications at the 3′ end of chemically synthesized pegRNAs. An experiment was conducted to explore two approaches for prime editing adopted from the literature that knock out the PAM in EMX1 or introduce a 3-base insertion in RUNX1, both of which utilize pegRNAs with a primer binding sequence comprising 15 nucleotides. The particular sequence edits that were evaluated in this experiment are shown in FIG. 18. An mRNA encoding a prime editor (in this case, a fusion protein comprising a Cas9 nickase and an MMLV-derived reverse transcriptase) was introduced into K562 or Jurkat cells with a pegRNA targeting the EMX1 gene. Each transfection was performed in triplicate samples of cells that were cultured separately. Genomic DNA was harvested, the EMX1 target sequence was amplified using primers specific for EMX1 to produce amplicons that were sequenced, and the extent of prime editing (“% Edit”) was determined from the sequencing results. Also determined from the sequencing results was the extent of undesired indel formation (“% Indels”) at the nickase site in the EMX1 target sequence. Such indels are known byproducts of prime editing and are generally considered undesirable (see Anzalone et al. 2019). Prime editing yields and indel byproduct yields per pegRNA are plotted as bar graphs in FIGS. 11-16. The sequences used in this assay were selected from sequences shown in Table 2. Data in FIGS. 11-12 were obtained using a first batch synthesis of pegRNAs targeting EMX1, whereas data in FIGS. 13-14 were obtained using a second batch synthesis of pegRNAs targeting EMX1. Note that some of the same sequences were synthesized again in the second batch synthesis. Conversely data in FIGS. 15-16 were obtained using pegRNAs targeting RUNX1 (i.e., using sequences described in Table 3).

As illustrated by the results shown in FIGS. 11-16, the inclusion of MS and MP nucleotides as chemical modifications at the 5′ end and the 3′ end, respectively, of pegRNAs increases prime editing activity. The enhanced activity of constructs having modified nucleotides at the 3′ end of the pegRNA is particularly surprising, given the fact that the 3′ end of a pegRNA contains additional functional sites (e.g., the primer binding site and a reverse transcriptase template sequence). As noted above, prior to the present disclosure it would have been expected that the inclusion of chemically-modified nucleotides (e.g., MS and/or MP) at this site would interfere with the functionality provided by these other 3′ end components of a pegRNA.

### Example 4

This example evaluated the incorporation of MP or MS modifications at the 3′ end of chemically synthesized pegRNAs. The methods used in this experiment are consistent with the methods described above. In short, prime editing approaches were adopted to knockout the PAM in EMX1 or to introduce a 3-base insertion in RUNX1. K562 cells were co-transfected with prime editor mRNA (in this case, a fusion protein comprising a Cas9 nickase and an MMLV-derived reverse transcriptase) and synthetic pegRNA modified by 3×MS at the 5′ end and various modification schemes at the 3′ end (as indicated) for editing EMX1 or RUNX1. Jurkat cells were likewise transfected using the same pegRNAs for editing EMX1 or RUNX1. Editing yields were measured by deep sequencing of PCR amplicons of the target loci for both the desired edit (% Edit) and any contaminating indel byproducts (% By-indels). Bars in the associated figures represent means with std. dev. (n=3).

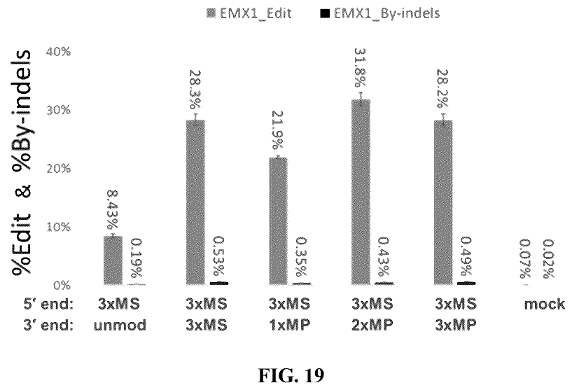

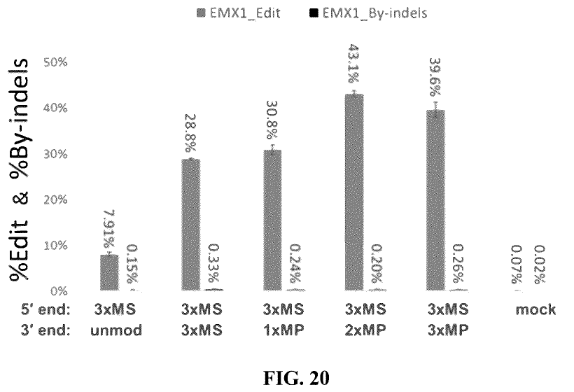

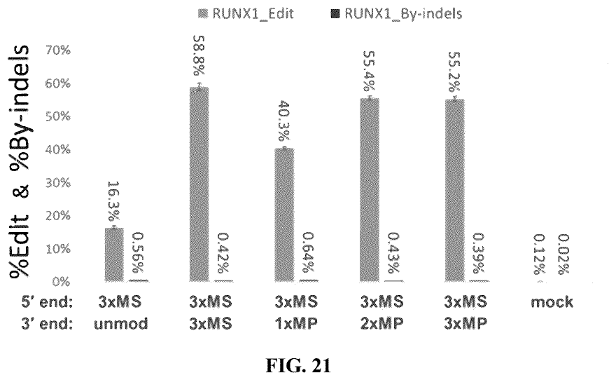

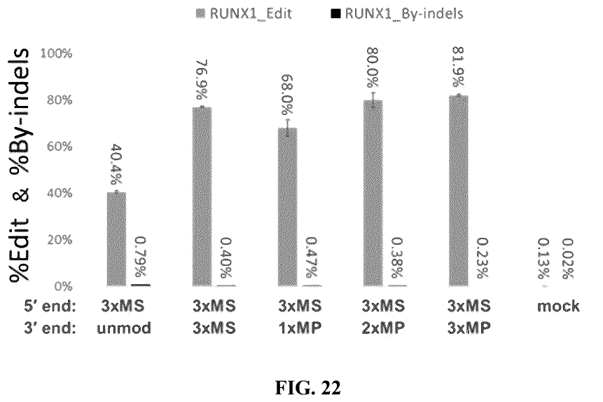

As shown by FIGS. 19-22, this experiment compared pegRNAs having 3×MS at the 3′ end for both targets with alternative designs having one, two or three consecutive MPs at the 3′ end, each co-transfected with PE2 mRNA in K562 or Jurkat cells. The results show that pegRNAs with MP modifications at the 3′ end performed well and can achieve comparable, or in some cases somewhat higher, editing yields than 3×MS. For the two pegRNA sequences tested here, designs with 2×MP and/or 3×MP at the 3′ end performed consistently better than designs with 1×MP at the 3′ end (specifically 1.2-1.4-fold better).

### Example 5

This example demonstrated that the use of MP modifications at the 3′ end of chemically synthesized gRNAs help to maximize editing yields in the presence of serum. To simulate harsher cellular environments that CRISPR-Cas components may encounter when delivered in vivo (as by nanocarriers or other cell-penetrating formulations), an experiment was conducted wherein Cas9 mRNA was co-transfected with gRNA into cells that were isolated from culture media but not rinsed with PBS buffer to remove residual serum, which is known to contain nucleases.

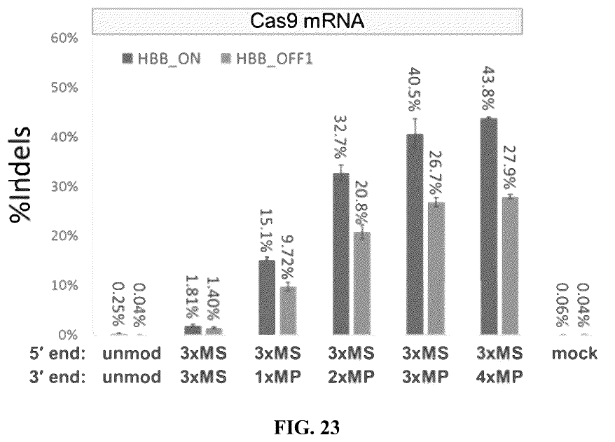

The methods used in this experiment are consistent with the methods described above. However, it is noted that under the conditions of this experiment, higher amounts of gRNA and Cas9 mRNA were needed to achieve substantial levels of editing, specifically, 3-fold more gRNA and 8-fold more Cas9 mRNA per transfection was used for the experiment that resulted in the data shown in FIG. 23, as compared to the experiment that resulted in the data shown in FIG. 3 where the same number of cells per transfection were washed with buffer before introducing the CRISPR-Cas components.

Based on the results of this study, it appears that extracellular nucleases in serum not rinsed from cells degraded the transfected RNAs. We found that gRNAs with MP modifications at the 3′ end gave substantially higher editing yields (by an order-of-magnitude or more) compared to gRNAs with MS modifications at the 3′ end when co-transfected with Cas9 mRNA into unrinsed HepG2 cells (FIG. 23). Specifically, 15%-44% editing yield for gRNAs with one or more MPs at the 3′ end was observed versus less than 2% with 3×MS at the 3′ end.

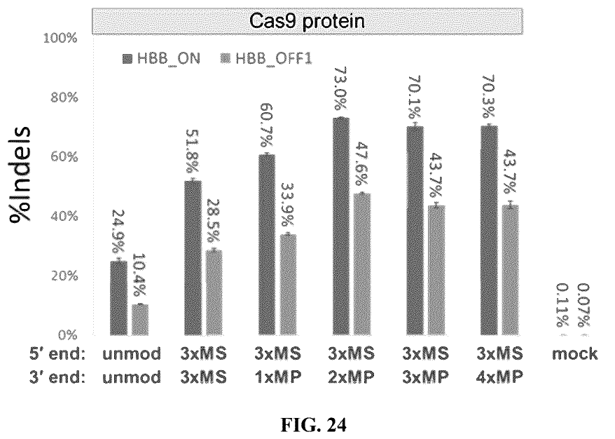

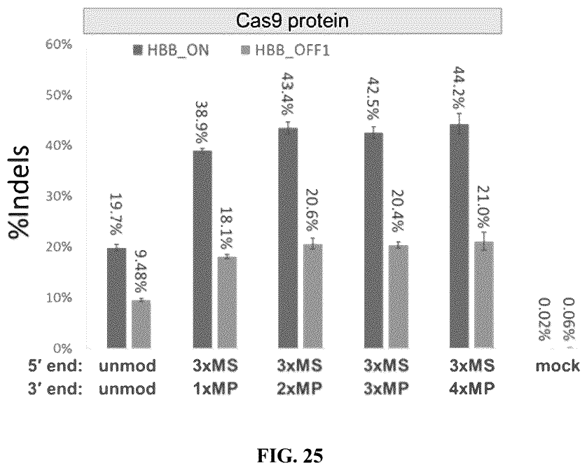

In a parallel experiment, an RNP version of each gRNA was prepared by pre-complexation with Cas9 protein in PBS buffer and transfected into aliquots of unrinsed HepG2 cells. As expected, the unmodified and 3×MS-modified gRNAs gave higher indel yields as RNP formulations than when these were co-transfected with Cas9 mRNA, as pre-complexation of gRNA with Cas9 protein in RNP helps shield the gRNA from nucleolytic degradation (compare the results shown in FIG. 24 versus FIG. 23). Even though the improvement in editing efficiency between RNPs incorporating gRNA with MP versus MS modifications at the 3′ end was not as dramatic as when using these modifications in co-transfections with Cas9 mRNA, designs with MPs at the 3′ end gave significantly higher Cas9-mediated indels than a comparable design with 3×MS at the 3′ end (70%-73% indels at the ON-target site for the 2×MP, 3×MP and 4×MP modifications at the 3′ end versus 52% for 3×MS at the 3′ end, a difference of about 1.3 fold) (see FIG. 24). A similar outcome was observed for a different set of synthetic 163mer gRNAs designed for CRISPRa SAM systems but used with SpCas9 protein in RNP formulations to produce indels instead of using them for gene activation by CRISPRa (FIG. 25).

### EXEMPLARY EMBODIMENTS

### Section A

Embodiment A1. A method of editing a target region in a nucleic acid under one or more challenging conditions, the method comprising:
providing to the cell
a) a CRISPR-associated (“Cas”) protein, and
b) a modified guide RNA comprising a guide sequence that is capable of hybridizing to the target region and a scaffold that interacts with the Cas protein, wherein the modified guide RNA comprises a 5′ end and a 3′ end, and the modified guide RNA further comprises one or more modified nucleotides within 5 nucleotides of the 3′ end, wherein the one or more modified nucleotides comprises at least one nucleotide with a 2′ modification and an internucleotide linkage modification, wherein the 2′ modification is selected from 2′-O-methyl, 2′-fluoro, 2′-O-methoxyethyl (2′-MOE) and 2′-deoxy, and the internucleotide linkage modification is a phosphonocarboxylate or thiophosphonocarboxylate;
wherein the one or more challenging conditions are selected from the group consisting of:
i. the target region or a cell comprising the target region is in a medium comprising serum (e.g., fetal bovine serum);
ii. a cell comprising the target region was previously cultured in a medium comprising serum, and the cell was incompletely separated from the serum;
iii. a cell comprising the target region was previously cultured in a medium comprising one or more exoribonucleases, and the cell was incompletely separated from the one or more exoribonucleases;
iv. a cell comprising the target region has a relatively high level of exoribonuclease activity, such as relatively high expression of one or more exoribonucleases;
v. a cell comprising the target region has a relatively low level of ribonuclease inhibitor activity, such as a relatively low expression of ribonuclease inhibitor;
vi. the modified guide RNA is not in a complex with a Cas protein before delivery into a cell comprising the target region; and
vii. applicable combinations thereof;
wherein the Cas protein and the modified guide RNA form a complex that results in editing the target region.

Embodiment A2. The method of Embodiment A1, wherein the internucleotide linkage modification is a phosphonocarboxylate.

Embodiment A3. The method of Embodiment A2, wherein the phosphonocarboxylate is phosphonoacetate.

Embodiment A4. The method of Embodiment A1, wherein the thiophosphonocarboxylate is thiophosphonoacetate.

Embodiment A5. The method of any of Embodiments A1 to 4, wherein the Cas protein is introduced as an mRNA encoding the Cas protein.

Embodiment A6. The method of any of Embodiments A1 to 4, wherein the Cas protein is introduced as an expression vector encoding the Cas protein.

Embodiment A7. The method of Embodiment A5 or A6, wherein the mRNA or expression vector encoding the Cas protein is contained in a nanoparticle when introduced to the target region.

Embodiment A8. The method of any of Embodiments A1 to A4, wherein the Cas protein and the guide RNA are introduced as a ribonucleoprotein (RNP) complex.

Embodiment A9. The method of any of the preceding embodiments, wherein the 2′ modification is 2′-O-methyl.

Embodiment A10. The method of any of Embodiments A1-A8, wherein the 2′ modification is 2′-fluoro.

Embodiment A11. The method of any of Embodiments A1-A8, wherein the 2′ modification is 2′-MOE.

Embodiment A12. The method of any of Embodiments A1-A8, wherein the 2′ modification is 2′-deoxy.

Embodiment A13. The method of any of the preceding embodiments, wherein the one or more edits comprise one or more single-nucleotide changes, an insertion of one or more nucleotides, and/or a deletion of one or more nucleotides.

Embodiment A14. The method of any of the preceding embodiments, wherein the target region is present in a cell-free assay.

Embodiment A15. The method of Embodiment A14, wherein the method further comprises extracting nucleic acid from a cell, such as by lysing the cell, forming an assay mixture comprising the extracted nucleic acid and one or more other cell components, such as exoribonucleases or other enzymes, and introducing the guide RNA to the assay mixture.

Embodiment A16. The method of any of the preceding embodiments, wherein the target region is in a cell having high ribonuclease expression, concentration and/or activity, for example, cell types high in a particular nuclease.

Embodiment A17. The method of Embodiment A16, wherein the cells comprise primary cells.

Embodiment A18. The method of Embodiment A17, wherein the cell exists ex vivo, and the method further comprises one or more steps for separating the cell from a living organism. The cell can be separated into a reaction mixture, or a separated cell can be transferred into a reaction mixture.

Embodiment A19. The method of any of Embodiments A16-A18, wherein the cell is isolated from a multicellular organism prior to introducing the modified guide RNA and the Cas protein to the target region in the cell.

Embodiment A20. The method of Embodiment A19, wherein the cell or a progeny thereof is returned to the multicellular organism after introducing the modified guide RNA and the Cas protein to the target region in the cell.

Embodiment A21. The method of any of Embodiments A16-A20, wherein the cell is a primary cell.

Embodiment A22. The method of Embodiment A21, wherein the primary cell is a stem cell or an immune cell.

Embodiment A23. The method of Embodiment A22, wherein the stem cell is a hematopoietic stem and progenitor cell (HSPC), a mesenchymal stem cell, a neural stem cell, or an organ stem cell.

Embodiment A24. The method of Embodiment A22, wherein the immune cell is a T cell, a natural killer cell, a monocyte, a peripheral blood mononuclear cell (PBMC), or a peripheral blood lymphocyte (PBL).

Embodiment A25. The method of Embodiment A24, wherein the cell is a T-cell.

Embodiment A26. The method of any of Embodiments A16-A20, wherein the cell is a hepatocyte.

Embodiment A27. The method of any of Embodiments A16-A26, wherein the cell is a population of cells, each comprising the target region.

Embodiment A28. The method of any of Embodiments A16-A27, wherein the cell is in a cell culture, wherein the cell is in a cell culture medium comprising serum or one or more other medium components.

Embodiment A29. The method of Embodiment A28, wherein the cell is not separated from the cell culture medium before the Cas protein and the modified guide RNA are introduced.

Embodiment A30. The method of any of Embodiments A1-13 and A16-A29, wherein the Cas protein and the modified guide RNA are introduced into a living organism.

Embodiment A31. The method of Embodiment A30, wherein the Cas protein and the modified guide RNA are introduced to a serum-containing fluid in or from the living organism.

Embodiment A32. The method of any of the preceding embodiments, wherein the editing is prime editing, and the modified guide RNA further comprises a region comprising desired edit(s).

Embodiment A33. The method of any of the preceding embodiments, wherein the editing comprises homologous-directed repair (HDR), nonhomologous end joining (NHEJ), prime editing, or base editing.

Embodiment A34. The method of any of the preceding embodiments, wherein the Cas protein is a Cas9 or Cas12 protein.

Embodiment A35. The method of any of the preceding embodiments, wherein the Cas protein is a Cas nickase capable of nicking a single strand of DNA.

Embodiment A36. The method of any of the preceding embodiments, wherein the Cas protein is a fusion protein comprising a Cas domain and a heterologous functional domain, wherein the heterologous functional domain comprises base editing activity, nucleotide deaminase activity, transglycosylase activity, methylase activity, demethylase activity, reverse transcriptase activity, polymerase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

Embodiment A37. The method of Embodiment A36, wherein the fusion protein comprises a Cas nickase domain and a nucleotide deaminase.

Embodiment A38. The method of Embodiment A36, wherein the nucleotide deaminase is an adenosine deaminase or a cytidine deaminase.

Embodiment A39. The method of Embodiment A36, wherein the fusion protein comprises one or more nucleic acid modifying domains.

Embodiment A40. The method of Embodiment A36, wherein the nucleic acid modifying domain is a DNA polymerase domain, a recombinase domain, a ribonucleotide reductase domain, a methyltransferase domain, a diadenosine tetraphosphate hydrolase domain, a DNA helicase domain, or a RNA helicase domain.

Embodiment A41. The method of Embodiment A36, wherein the fusion protein comprises a Cas nickase domain and a reverse transcriptase domain.

Embodiment A42. The method of any of the preceding embodiments, wherein the guide RNA is a single-guide RNA.

Embodiment A43. The method of Embodiment A42, wherein the modified guide RNA is a single-guide RNA comprising at least 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, or 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 nucleotides, and/or
up to 180, 179, 178, 177, 176, 175, 174, 173, 172, 171, 170, 169, 168, 167, 166, 165, 164, 163, 162, 161, 159, 158, 157, 156, 155, 154, 153, 152, 151, 150, 149, 148, 147, 146, 145, 144, 143, 142, 141, 140, 139, 138, 137, 136, 135, 134, 133, 132, 131, 130, 129, 128, 127, 126, 125, 124, 123, 122, 121, or 120 nucleotides.

Embodiment A44. The method of any of the preceding embodiments, wherein the guide RNA further comprises one or more modified nucleotides within 5 nucleotides of the 5′ end, alternatively within 3 nucleotides of the 5′ end.

Embodiment A45. The method of Embodiment A44, wherein the one or more modified nucleotides at the 5′ end comprises at least one nucleotide with a 2′ modification and an internucleotide linkage modification, wherein the 2′ modification is selected from 2′-O-methyl, 2′-fluoro, 2′-O-methoxyethyl (2′-MOE) and 2′-deoxy, and the internucleotide linkage modification is selected from phosphonocarboxylate, thiophosphonocarboxylate, and phosphorothioate.

Embodiment A46. The method of any of the preceding embodiments, wherein the guide RNA further comprises one or more modified nucleotides at one or more positions other than at least 5 nucleotides from both the 5′ end and the 3′ end of the guide RNA.

Embodiment A47. A method of modulating expression of a target gene in a target region, in a nucleic acid in a cell, under one or more challenging conditions, the method comprising:
providing to the cell
a) a CRISPR-associated (“Cas”) protein, or a DNA or mRNA encoding the Cas protein, and
b) a modified guide RNA comprising a guide sequence that is capable of hybridizing to the target region and a region that interacts with the Cas protein, wherein the modified guide RNA comprises a 5′ end and a 3′ end, and the modified guide RNA further comprises one or more modified nucleotides within 5 nucleotides of the 3′ end, wherein the one or more modified nucleotides comprises at least one nucleotide with a 2′ modification and an internucleotide linkage modification, wherein the 2′ modification is selected from 2′-O-methyl, 2′-fluoro, 2′-O-methoxyethyl (2′-MOE) and 2′-deoxy, and the internucleotide linkage modification is a phosphonocarboxylate or thiophosphonocarboxylate; and
wherein the Cas protein and the modified guide RNA form a complex that results in modulating the expression of the target region.

Embodiment A48. The method of Embodiment A47, wherein the Cas protein or the modified guide RNA further comprise an epigenetic modifier, or a transcriptional or translational activation or repression signal.

Embodiment A49. The method of Embodiment A47, wherein the Cas protein is a fusion protein comprising an inactive Cas nuclease domain and a heterologous functional domain selected from a transcriptional activation domain and a transcriptional repression domains.

Embodiment A50. The method of Embodiment A49, wherein the heterologous functional domain is a transcriptional activation domain.

Embodiment A51. The method of Embodiment A50, wherein the transcriptional activation domain is a VP64 domain, a p65 domain, a MyoD1 domain, or a HSF1 domain.

Embodiment A52. The method of Embodiment A49, wherein the heterologous functional domain is a transcriptional repression domain.

Embodiment A53. The method of Embodiment A52, wherein the transcriptional repression domain is a KRAB domain, a SID domain, a SID4X domain, a NuE domain, or a NcoR domain.

Embodiment A54. A method of prime editing a target region in a nucleic acid under one or more challenging conditions, the method comprising:
a) providing to the cell
   a Cas protein capable of nicking a single strand of the nucleic acid;
   a reverse transcriptase; and
   a modified prime editing guide RNA (“pegRNA”) comprising:
   i) a guide sequence that is capable of hybridizing to the target region,
   ii) a region that interacts with the Cas protein,
   iii) a reverse transcriptase template sequence that comprises one or more edits to the sequence of the nucleic acid, and
   iv) a primer-binding site sequence that can bind to a complement of the target region; wherein the modified pegRNA comprises a 5′ end and a 3′ end, and the modified pegRNA further comprises one or more modified nucleotides within 5 nucleotides of the 3′ end, wherein the one or more modified nucleotides comprises at least one nucleotide with a 2′ modification and an internucleotide linkage modification, wherein the 2′ modification is selected from 2′-O-methyl, 2′-fluoro, 2′-O-methoxyethyl (2′-MOE) and 2′-deoxy, and the internucleotide linkage modification is a phosphonocarboxylate or thiophosphonocarboxylate; and
wherein the Cas protein and the modified guide RNA form a complex that results in editing the target region.

Embodiment A55. The method of Embodiment A54, wherein the Cas protein and the reverse transcriptase are connected by a linker to form a fusion protein.

Embodiment A56. The method of any one of the preceding embodiments, wherein the guide RNA comprises at least one phosphorothioate internucleotide linkage within 5 nucleotides of the 5′ end, and at least two consecutive phosphonocarboxylate or thiophosphonocarboxylate internucleotide linkages within 5 nucleotides of the 3′ end.

Embodiment A57. The method of any one of the preceding embodiments, wherein the guide RNA comprises at least one phosphorothioate internucleotide linkage within 5 nucleotides of the 5′ end, and at least two consecutive phosphonoacetate or thiophosphonoacetate internucleotide linkages within 5 nucleotides of the 3′ end.

Embodiment A58. The method of any one of the preceding embodiments, wherein the guide RNA comprises at least one MS within 5 nucleotides of the 5′ end, and at least two consecutive MP or MSP within 5 nucleotides of the 3′ end.

Embodiment A59. The method of any one of the preceding embodiments, wherein the guide RNA comprises three MS within 5 nucleotides of the 5′ end, and three MP or MSP within 5 nucleotides of the 3′ end.

Embodiment A60. The method of any one of the preceding embodiments, wherein the editing and/or the modulation of expression of a target gene are performed in a multiplexed fashion (i.e. on at least two target genes or at least two target regions).

### Section B

Embodiment B1. A method of editing a target region in a nucleic acid in a cell, the method comprising providing to the cell:
a) a CRISPR-associated (“Cas”) protein, and
b) a modified guide RNA comprising a 5′ end and a 3′ end, and:
   a guide sequence that is capable of hybridizing to a target sequence in the target region,
   a scaffold region that interacts with the Cas protein, and
   one or more phosphorothioate modifications within 5 nucleotides of the 5′ end, and at least two consecutive phosphonocarboxylate or thiophosphonocarboxylate modifications within 5 nucleotides of the 3′ end;
wherein the cell exists ex vivo in the presence of a nuclease-containing fluid, or exists in vivo, and
said providing results in editing of the target region.

Embodiment B1.1. A method of editing a target region in a nucleic acid in a cell, the method comprising providing to the cell:
a) a CRISPR-associated (“Cas”) protein, and
b) a modified guide RNA that is a prime editing guide RNA (pegRNA) comprising a 5′ end and a 3′ end, one of which is a prime editing end and the other is a distal end, the modified guide RNA further comprising:
a guide sequence that is capable of hybridizing to a target sequence in the target region,
a scaffold region that interacts with the Cas protein, and
one or more phosphorothioate modifications within 5 nucleotides of the distal end, and at least two consecutive phosphonocarboxylate or thiophosphonocarboxylate modifications within 5 nucleotides of the prime editing end;
wherein the cell exists ex vivo in the presence of a nuclease-containing fluid, or exists in vivo, and
said providing results in editing of the target region.

Embodiment B2. The method of Embodiment B1 or B1.1, wherein said editing occurs with an efficiency higher than that by an unmodified gRNA that is otherwise identical to the modified guide RNA

Embodiment B3. A method of modulating expression of a target gene in a target region in a nucleic acid in a cell, the method comprising providing to the cell:
a) a CRISPR-associated (“Cas”) protein, and
b) a modified guide RNA comprising a 5′ end and a 3′ end, and:
   a guide sequence that is capable of hybridizing to a target sequence in the target region,
   a scaffold region that interacts with the Cas protein, and
   one or more phosphorothioate modifications within 5 nucleotides of the 5′ end, and at least two consecutive phosphonocarboxylate or thiophosphonocarboxylate modifications within 5 nucleotides of the 3′ end;
wherein the cell exists ex vivo in the presence of a nuclease-containing fluid, or exists in vivo, and
said providing results in modulation of expression of the target gene.

Embodiment B4. The method of Embodiment B3, wherein the modulation occurs with an efficiency higher than that by an unmodified gRNA that is otherwise identical to the modified guide RNA.

Embodiment B5. The method of any one of the preceding B embodiments, wherein the cell exists in vivo.

Embodiment B6. The method of any one of the preceding B embodiments, wherein the cell exists ex vivo in the presence of a nuclease-containing fluid.

Embodiment B7. The method of any one of the preceding B embodiments, wherein the modified guide RNA comprises at least two consecutive 2′-O-methyl-3′-phosphorothioate (MS) within 5 nucleotides of the 5′ end (exception; “the distal end” in lieu of “the 5′ end” when this embodiment depends from Embodiment B1.1).

Embodiment B8. The method of any one of the preceding B embodiments, wherein the phosphonocarboxylate is phosphonoacetate and the thiophosphonocarboxylate is thiophosphonoacetate.

Embodiment B9. The method of any one of the preceding B embodiments, wherein the modified guide RNA comprises at least two consecutive 2′-O-methyl-3′-phosphonoacetate (MP) or 2′-O-methyl-3′-thiophosphonoacetate (MSP) within 5 nucleotides of the 3′ end (exception: “the prime ending end” in lieu of “the 5′ end” when this embodiment depends from Embodiment B 1.1).

Embodiment B10. The method of any one of the preceding B embodiments, wherein the modified guide RNA further comprises modified nucleotide(s) located outside of 5 nucleotides within the 5′ end and 3′ end.

Embodiment B11. The method of any one of the preceding B embodiments, wherein the modified guide RNA is a single guide RNA.

Embodiment B12. The method of any one of the preceding B embodiments, wherein the Cas protein is provided as an mRNA encoding the Cas protein.

Embodiment B13. The method of any one of Embodiments B1-B11, wherein the Cas protein is provided as a DNA encoding the Cas protein.

Embodiment B14. The method of Embodiment B13, wherein the DNA is a viral expression vector.

Embodiment B15. The method of any one of Embodiments B1-11, wherein the Cas protein and the modified guide RNA are provided as a ribonucleoprotein complex (RNP).

Embodiment B16. The method of any one of Embodiments B1-B11, wherein the Cas protein and/or modified guide RNA are provided in nanoparticle(s).

Embodiment B17. The method of any one of the preceding B embodiments, wherein the efficiency is higher by at least 5%.

Embodiment B18. The method of any one of the preceding B embodiments, wherein the efficiency is higher by at least 10%.

Embodiment B19. The method of any one of the preceding B embodiments, wherein the efficiency is higher by at least 15%.

Embodiment B20. The method of any one of the preceding B embodiments, wherein the efficiency is higher by at least 20%.

Embodiment B21. The method of any one of the preceding B embodiments, wherein the efficiency is higher by at least 25%.

Embodiment B22. The method of any one of the preceding B embodiments, wherein the efficiency is higher by at least 30%.

Embodiment B23. The method of any one of the preceding B embodiments, wherein the efficiency is higher by at least 35%.

Embodiment B24. The method of any one of the preceding B embodiments, wherein the efficiency is higher by at least 40%.

Embodiment B25. The method of any one of the preceding B embodiments, wherein the efficiency is higher by at least 45%.

Embodiment B26. The method of any one of the preceding B embodiments, wherein the efficiency is higher by at least 50%.

Embodiment B27. The method of any one of the preceding B embodiments, wherein the Cas protein is capable of cleaving both strands of DNA.

Embodiment B28. The method of any one of embodiments B1-B26, wherein the Cas protein is a nickase.

Embodiment B29. The method of any one of embodiments B1-B26, wherein the Cas protein does not have nuclease activity.

Embodiment B30. The method of any one of the preceding B embodiments, wherein the Cas protein is part of a fusion protein that further comprises a heterologous protein.

Embodiment B31. The method of any one of the preceding B embodiments, wherein the Cas protein is a Type II Cas protein.

Embodiment B32. The method of any one of the preceding B embodiments, wherein the Cas protein is a Cas9 protein, or a variant or fragment thereof.

Embodiment B33. The method of Embodiment B32, wherein the Cas9 protein is from Streptococcus pyogenes.

Embodiment B34. The method of any one of Embodiments B1-B32, wherein the Cas protein is a Cpf1 protein, or a variant or fragment thereof.

Embodiment B35. The method of any one of the preceding B embodiments, wherein the Cas protein is a hybrid protein having sequences from at least two different wild type Cas proteins.

Embodiment B36. The method of any one of the preceding B embodiments, wherein the modified guide RNA is 40-70 nucleotides in length.

Embodiment B37. The method of any one of the preceding B embodiments, wherein the modified guide RNA is 40-100 nucleotides in length.

Embodiment B38. The method of any one of Embodiments B1-B35, wherein the modified guide RNA is 90-110 nucleotides in length.

Embodiment B39. The method of any one of Embodiments B1-B35, wherein the modified guide RNA is 90-130 nucleotides in length.

Embodiment B40. The method of any one of Embodiments B1-B35, wherein the modified guide RNA is 130-160 nucleotides in length.

Embodiment B41. The method of any one of Embodiments B1-B35, wherein the modified guide RNA is 160-200 nucleotides in length.

Embodiment B42. The method of any one of the preceding B embodiments, wherein the modified guide RNA is a pegRNA.

Embodiment B43. The method of any one of the preceding B embodiments, wherein the phosphorothioate, phosphonocarboxylate or thiophosphonocarboxylate modifications are each present in a nucleotide that also comprises a 2′-O-Methyl modification.

Embodiment B44. The method of any one of the preceding B embodiments, further comprising editing a second target region in the cell using a second modified guide RNA that comprises:
a 5′ end and a 3′ end,
a guide sequence that is capable of hybridizing to a second target sequence in the second target region, and
one or more phosphorothioate modifications within 5 nucleotides of the 5′ end (except that if this embodiment depends from B1.1, this would be the distal end), and at least two consecutive phosphonocarboxylate or thiophosphonocarboxylate modifications within 5 nucleotides of the 3′ end (except that if this embodiment depends from B1.1, this would be the prime editing end).

Embodiment B45. The method of any one of the preceding B embodiments, further comprising modulating expression of a third target gene in a third target region in the cell using a third modified guide RNA that comprises:
a 5′ end and a 3′ end,
a guide sequence that is capable of hybridizing to a third target sequence in the third target region, and
one or more phosphorothioate modifications within 5 nucleotides of the 5′ end, and at least two consecutive phosphonocarboxylate or thiophosphonocarboxylate modifications within 5 nucleotides of the 3′ end.

Embodiment B46. The method of any one of the preceding B embodiments, wherein the nuclease is an exonuclease.

Embodiment B47. The method of any one of the preceding B embodiments, wherein the nuclease is ribonuclease.

### Section C

Embodiment C1. A method of editing two or more nucleic acid target regions, comprising a first target region and a second target region in a cell, the method comprising providing to the cell:
a) a CRISPR-associated (“Cas”) protein;
b) a first modified guide RNA comprising a 5′ end and a 3′ end, and:
   a first guide sequence that is capable of hybridizing to a first target sequence in the first target region,
   a scaffold region that interacts with the Cas protein, and
   one or more phosphorothioate modifications within 5 nucleotides of the 5′ end, and at least two consecutive phosphonocarboxylate or thiophosphonocarboxylate modifications within 5 nucleotides of the 3′ end;
c) a second modified guide RNA comprising a 5′ end and a 3′ end, and:
   a second guide sequence that is capable of hybridizing to a second target sequence in the second target region,
   a scaffold region that interacts with the Cas protein, and
   one or more phosphorothioate modifications within 5 nucleotides of the 5′ end, and
at least two consecutive phosphonocarboxylate or thiophosphonocarboxylate modifications within 5 nucleotides of the 3′ end;
wherein the cell exists ex vivo in the presence of a nuclease-containing fluid, or exists in vivo, and
said providing results in editing of the first and second target regions.

Embodiment C2. A method of modulating expression of at least a first target gene in a first target region and a second target gene in a second target region in a cell, the method comprising providing to the cell:
a) a CRISPR-associated (“Cas”) protein;
b) a first modified guide RNA comprising a 5′ end and a 3′ end, and:
   a first guide sequence that is capable of hybridizing to a first target sequence in the first target region,
   a scaffold region that interacts with the Cas protein, and
   one or more phosphorothioate modifications within 5 nucleotides of the 5′ end, and at least two consecutive phosphonocarboxylate or thiophosphonocarboxylate modifications within 5 nucleotides of the 3′ end;
c) a second modified guide RNA comprising a 5′ end and a 3′ end, and:
   a second guide sequence that is capable of hybridizing to a second target sequence in the second target region,
   a scaffold region that interacts with the Cas protein, and
   one or more phosphorothioate modifications within 5 nucleotides of the 5′ end, and at least two consecutive phosphonocarboxylate or thiophosphonocarboxylate modifications within 5 nucleotides of the 3′ end;
wherein the cell exists ex vivo in the presence of a nuclease-containing fluid, or exists in vivo, and
said providing results in modulation of expression of the first and second target genes.

Embodiment C3. The method of Embodiment C1 or C2, wherein the editing of the first target region, or modulation of the first target gene, has a first efficiency which is higher than that of an unmodified guide RNA otherwise identical to the first modified guide RNA.

Embodiment C4. The method of Embodiment C3, wherein the editing of the second target region, or modulation of the second target gene, has a second efficiency which is higher than that of an unmodified guide RNA otherwise identical to the second modified guide RNA.

Embodiment C5. The method of any one of the preceding C embodiments, wherein the cell exists in vivo.

Embodiment C6. The method of any one of Embodiment C1-C4, wherein the cell exists ex vivo in the presence of a nuclease-containing fluid.

Embodiment C7. The method of any one of the preceding C embodiments, further comprising applicable additional limitation(s) from each of the A embodiments or B embodiments.

The foregoing description of exemplary or preferred embodiments should be taken as illustrating, rather than as limiting, the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

All publications, patents, and patent applications mentioned in this specification are herein incorporated by reference to the same extent as if each individual publication, patent, or patent application was specifically and individually indicated to be incorporated by reference.

We claim:

1. A method of base editing a target region in a nucleic acid in a cell, the method comprising:
providing to the cell:
mRNA encoding a fusion protein wherein the fusion protein comprises a deaminase and a Cas protein capable of nicking a single strand of the nucleic acid; and
a modified guide RNA comprising
a 5′ end and a 3′ end;
a guide sequence that is capable of hybridizing to a target sequence in the target region;
a scaffold region that interacts with the Cas protein; and
one or more phosphorothioate modifications within 5 nucleotides of the 5′ end and at least one phosphonocarboxylate or thiophosphonocarboxylate modification within 5 nucleotides of the 3′ end;
wherein the cell exists ex vivo in the presence of a nuclease-containing fluid, or exists in vivo, and
said method results in base editing of the target region.

2. The method of claim 1, wherein the fusion protein comprises a cytosine deaminase domain or an adenine deaminase domain.

3. The method of claim 1, wherein the phosphorothioate, phosphonocarboxylate and thiophosphonocarboxylate modifications are each present in a nucleotide that also comprises 2′-O-methyl.

4. The method of claim 1, wherein the modified guide RNA comprises at least two consecutive 2′-O-methyl-3′-phosphorothioate (MS) within 5 nucleotides of the 5′ end.

5. The method of claim 1, wherein the phosphonocarboxylate is phosphonoacetate and the thiophosphonocarboxylate is thiophosphonoacetate.

6. The method of claim 4, wherein the modified guide RNA comprises at least two consecutive 2′-O-methyl-3′-phosphonoacetate (MP) or 2′-O-methyl-3′-thiophosphonoacetate (MSP) within 5 nucleotides of the 3′ end.

7. The method of claim 4, wherein the modified guide RNA comprises at least three consecutive 2′-O-methyl-3′-phosphonoacetate (MP) or 2′-O-methyl-3′-thiophosphonoacetate (MSP) within 5 nucleotides of the 3′ end.

8. The method of claim 4, wherein the modified guide RNA comprises at least three consecutive 2′-O-methyl-3′-phosphonoacetate (MP) or 2′-O-methyl-3′-thiophosphonoacetate (MSP) within 5 nucleotides of the 3′ end and wherein the fusion protein comprises a cytosine deaminase domain.

9. The method of claim 1, wherein the modified guide RNA further comprises modified nucleotide(s) located outside of 5 nucleotides within the 5′ end and 3′ end.

10. The method of claim 1, wherein the modified guide RNA comprises a phosphonocarboxylate or thiophosphonocarboxylate modification at nucleotide position 5 or nucleotide position 11, wherein the modified nucleotide position is counted from the 5′ end of the guide RNA.

11. The method of claim 1, wherein the modified guide RNA and the mRNA encoding a fusion protein are provided at a modified guide RNA:mRNA ratio of 100:1 or less.

12. The method of claim 1, wherein the modified guide RNA is a single guide RNA.

13. The method of claim 1, wherein the Cas protein and the modified guide RNA form a ribonucleoprotein complex (RNP).

14. The method of claim 1, wherein the mRNA encoding a fusion protein and/or modified guide RNA are provided in a nanoparticle.

15. The method of claim 1, wherein the base editing occurs with an efficiency higher than that by an unmodified guide RNA which is otherwise identical to the modified guide RNA.

16. The method of claim 15, wherein said efficiency is higher by at least 10% or at least 20%.

17. The method of claim 1, wherein the nuclease-containing fluid is selected from the group consisting of serum, cerebrospinal fluid (CSF), and cell culture medium.

18. The method of claim 1, wherein the cell exists ex vivo in the presence of nuclease-containing fluid, and the cell is not washed prior to providing the modified guide RNA to the cell.

19. The method of claim 1, wherein the cell exists in vivo in the presence of a nuclease-containing fluid.

20. The method of claim 1, wherein the cell is transfected with less than or equal to 800 pmol of modified gRNA per 1 million cells, less than or equal to 400 pmol of modified gRNA per 1 million cells, or less than or equal to 200 pmol of modified gRNA per 1 million cells.
